(19) United States
(12) Patent Application Publication
(10) Pub. No.: US 2005/0118715 A1
(43) Pub. Date: Jun. 2, 2005

(54) Modulation of stem and progenitor cell differentiation, assays, and uses thereof

(76) Inventors: Robert J. Hariri, Florham Park, NJ (US); David I. Stirling, Warren, NJ (US); Kyle W.H. Chan, San Diego, CA (US); Laure A. Moutouh-de Parseval, San Diego, CA (US)

Correspondence Address: JONES DAY, 222 EAST 41ST ST, NEW YORK, NY, 10017, US

(21) Appl. No.: 10/511,355

(22) PCT Filed: Apr. 11, 2003
(86) PCT No.: PCT/US03/11190

Related U.S. Application Data
(60) Provisional application No. 60/372,348, filed on Apr. 12, 2002.
(60) Provisional application No. 60/384,251, filed on May 30, 2002.
(60) Provisional application No. 60/437,350, filed on Dec. 31, 2002.
(60) Provisional application No. 60/437,348, filed on Dec. 31, 2002.

(51) Int. Cl. A61K 38/00; C12N 5/00; C12N 5/02

(52) U.S. Cl. 435/375; 514/2; 435/325

(57) ABSTRACT

The present invention relates to methods of modulating mammalian stem cell and progenitor cell differentiation. The methods of the invention can be employed to regulate and control the differentiation and maturation of mammalian, particularly human stem cells along specific cell and tissue lineages. The methods of the invention relate to the use of certain small organic molecules to modulate the differentiation of stem or progenitor cell populations along specific cell and tissue lineages, and in particular, to the differentiation of embryonic-like stem cells originating from a postpartum placenta or for the differentiation of early progenitor cells to a granulocytic lineage. Finally, the invention relates to the use of such differentiated stem or progenitor cells in transplantation and other medical treatments.

This application claims benefit of U.S. Provisional Application Nos. 60/372,348, filed Apr. 12, 2002; 60/384,251, filed May 30, 2002, 60/437,348, filed Dec. 31, 2002; and 60/437,350, filed Dec. 31, 2002, each of which is incorporated herein in its entirety.

### 1. INTRODUCTION

The present invention relates to methods of modulating mammalian stem and/or progenitor cell differentiation. The methods of the invention can be employed to regulate and control the differentiation and maturation of mammalian, particularly human, stem and progenitor cells along specific cell and tissue lineages. The methods of the invention relate to the use of certain small organic molecules to modulate the differentiation of stem cell populations along specific cell and tissue lineages, and in particular, to the differentiation of embryonic-like stem cells originating from a postpartum placenta or the modulation of early hematopoietic progenitor cells along a specific differentiation pathway, particularly a granulocytic differentiation pathway. The invention also relates to the use of these organic molecules to modulate the differentiation of particular lineages of progenitor cells, such as CD34+, CD45+ and CD 133+progenitor cells. The invention also relates to the temporal aspects of progenitor cell development, and in vitro models based upon these temporal aspects. The invention further relates to the use of these modulated cells in prophylactic and therapeutic methods, including in pharmaceutical compositions of such cells and/or small organic compounds. Finally, the invention relates to the use of such differentiated cells in transplantation and other medical treatments.

### 2. BACKGROUND OF THE INVENTION

There is considerable interest in the identification, isolation and generation of human stem and progenitor cells. Stem cells are totipotential or pluripotential precursor cells capable of generating a variety of mature cell lineages, and precursor cells are cells capable of generating cells of specific cell lineages. These abilities serve as the basis for the cellular differentiation and specialization necessary for organ and tissue development.

Recent success at translanting stem and progenitor cells have provided new clinical tools to reconstitute and/or supplement bone marrow after myeloablation due to disease, exposure to toxic chemical and/or radiation. Further evidence exists that demonstrates that stem cells can be employed to repopulate many, if not all, tissues and restore physiologic and anatomic functionality. The application of stem cells in tissue engineering, gene therapy delivery and cell therapeutics is also advancing rapidly.

Many different types of mammalian and progenitor stem cells have been characterized. For example, embryonic stem cells, embryonic germ cells, adult stem cells or committed stem cells or progenitor cells are known. Certain stem cells have not only been isolated and characterized but have also been cultured under conditions to allow differentiation to a limited extent. However, a basic problem remains; that is, it has been difficult to control or regulate the differentiation of stem cells and progenitor cells, such as hematopoietic progenitor cells. Presently, existing methods of modulating the differentiation of these cells are crude and unregulatable, such that the cells differentiate into unwanted cell types, at unwanted times. Moreover, the yield of the product cells is typically low.

Furthermore, obtaining sufficient numbers of human stem cells for therapeutic or research purposes is problematic. Isolation of normally occurring populations of stem or progenitor cells in adult tissues has been technically difficult and costly, due, in part, to the limited quantity of stem or progenitor cells found in blood or tissue, and the significant discomfort involved in obtaining bone marrow aspirates. In general, harvesting of stem or progenitor cells from alternative sources in adequate amounts for therapeutic and research purposes is generally laborious, involving, e.g., harvesting of cells or tissues from a donor subject or patient, culturing and/or propagation of cells in vitro, dissection, etc. With respect to stem cells in particular, procurement of these cells from embryos or fetal tissue, including abortuses, has raised religious and ethical concerns. The widely held belief that the human embryo and fetus constitute independent life has prompted governmental restrictions on the use of such sources for all purposes, including medical research. Alternative sources that do not require the use of cells procured from embryonic or fetal tissue are therefore desired for further progress in the use of stem cells clinically. There are, however, few viable alternative sources of stem or progenitor cells, particularly human stem or progenitor cells, and thus the supply is limited.

Hu et al. (WO 00/73421 entitled “Methods of isolation, cryopreservation, and therapeutic use of human amniotic epithelial cells,” published Dec. 7, 2000) discloses human amniotic epithelial cells derived from placenta at delivery that are isolated, cultured, cryopreserved for future use, or induced to differentiate. According to Hu et al., a placenta is harvested immediately after delivery and the amniotic membrane separated from the chorion, e.g., by dissection. Amniotic epithelial cells are isolated from the amniotic membrane according to standard cell isolation techniques. The disclosed cells can be cultured in various media, expanded in culture, cryopreserved, or induced to differentiate. Hu et al. discloses that amniotic epithelial cells are multipotential (and possibly pluripotential), and can differentiate into epithelial tissues such as corneal surface epithelium or vaginal epithelium. The drawback of such methods, however, is that they are labor-intensive and the yield of stem cells is very low.

Currently available methods for the ex vivo expansion of cell populations are also labor-intensive. For example, Emerson et al. (Emerson et al., U.S. Pat. No. 6,326,198 entitled “Methods and compositions for the ex vivo replication of stem cells, for the optimization of hematopoietic progenitor cell cultures, and for increasing the metabolism; GM-CSF secretion and/or IL-6 secretion of human stromal cells”, issued Dec. 4, 2001); discloses methods, and culture media conditions for ex vivo culturing of human stem cell division and/or the optimization of human hematopoietic progenitor stem cells. According to the disclosed methods, human stem cells or progenitor cells derived from bone marrow are cultured in a liquid culture medium that is replaced, preferably perfused, either continuously or periodically, at a rate of 1 ml of medium per ml of culture per about 24 to about 48 hour period. Metabolic products are removed and depleted nutrients replenished while maintaining the culture under physiologically acceptable conditions.

Kraus et al. (Kraus et al., U.S. Pat. No. 6,338,942, entitled “Selective expansion of target cell populations,” issued Jan. 15, 2002) discloses that a predetermined target population of cells may be selectively expanded by introducing a starting sample of cells from cord blood or peripheral blood into a growth medium, causing cells of the target cell population to divide, and contacting the cells in the growth medium with a selection element comprising binding molecules with specific affinity (such as a monoclonal antibody for CD34) for a predetermined population of cells (such as CD34 cells), so as to select cells of the predetermined target population from other cells in the growth medium.

Rodgers et al. (U.S. Pat. No. 6,335,195 entitled “Method for promoting hematopoietic and mesenchymal cell proliferation and differentiation,” issued Jan. 1, 2002) discloses methods for ex vivo culture of hematopoietic and mesenchymal stem cells and the induction of lineage-specific cell proliferation and differentiation by growth in the presence of angiotensinogen, angiotensin I (AI), AI analogues, AI fragments and analogues thereof, angiotensin II (A>1), All analogues, All fragments or analogues thereof or All AT₂ type 2 receptor agonists, either alone or in combination with other growth factors and cytokines. The stem cells are derived from bone marrow, peripheral blood or umbilical cord blood. The drawback of such methods, however, is that such ex vivo methods for inducing proliferation and differentiation of stem cells are time-consuming, as discussed above, and also result in low yields of stem cells.

Stem and progenitor cells have the potential to be used in the treatment of a variety of disorders, including malignancies, inborn errors of metabolism, hemoglobinopathies, and immunodeficiencies. One major area of use and research involving stem cells from cord blood or placenta has been the use of such cells to generate small quantities of cells for bone marrow and other related transplantations. However, to date, no one has described a method of producing substantial numbers of stem or progenitor cells, such as human CD34⁺ or CD133⁺ progenitor cells. Large numbers of the latter cells, in particular, would facilitate treatment methods using progenitor cells. The methods of the invention disclosed herein addresses this need.

Retinoids, such as vitamin A and retinoic acid (RA), have been known to affect differentiation of stem cells. For example, retinoic acid has been shown to inhibit proliferation of abnormally committed (chronic myelogenous leukemia) hematopoietic stem cells (Nadkarni et al. 1984, Tumori 70:503-505) and to induce differentiation and loss of self-renewal potential in promyelocytic leukemia cells (Melchner et al., 1985, Blood 66(6):,1469-1472). Retinoic acid has also been shown to induce differentiation of neurons from embryonic stem cells and to repress spontaneous mesodermal differentiation (Slager et al., Dev. Genet. 1993;14(3):212-24, Ray et al., 1997, J. Biol. Chem. 272(30): 18702-18708). Retinoic acid has further been shown to induce differentiation of transformed germ cell precursors (Damjanov et al., 1993, Labor. Investig. 68(2):220-232), placental cell precursors (Yan et al., 2001, Devel. Biol. 235: 422-432), and endothelial cell precursors (Hatzopoulos et al., 1998, Development 125: 1457-1468). The effect of retinoids on differentiation, however, has yet to be completely understood such that it could be used as a regulatable means of controlling differentiation of stem cells.

The effects of folic acid analogues, such as aminopterin and amethopterin (methotrexate), on the differentiation of hematopoietic stem cells has been studied. Folic acid analogues are used as chemotherapeutic agents in acute lymphoblastic anemias and other blood proliferation disorders and cancers, and have been shown to effect differentiation of stem cells by killing off certain populations of stem cells (DeLoia et al., 1998, Human Reproduction 13(4):1063-1069), and thus, would not be an effective tool for regulating differentiation of large quantities of stem cells for administration to a patient.

Several cytokines, such as IL-1, IL-2, IL-3, IL-6, IL-7, I[11, as well as proteins such as erythropoietin, Kit ligand, M-CSF and GM-CSF have also been shown to direct differentiation of stem cells into specific cell types in the hematopoietic lineage (Dushnik-Levinson et al., 1995, Biol. Neonate 67:77-83), however, these processes are not well understood and still remain too crude and imprecise to allow for a regulatable means of controlling differentiation of stem cells.

To date, no one has described the use of compounds, such as the PDE IV inhibitors discussed below, in the differentiation of stem cells or precursor cells. In particular, no one has demonstrated the use of such compounds to modulate the differentiation of progenitor cells, such as CD34⁺ progenitor cells, away from a dendritic cell lineage, a capability useful in encouraging transplant immune tolerance. Likewise, no one has described the use of the compounds described herein to expand the progenitor cell populations so as to produce a pharmaceutical composition containing such cells. Such expanded progenitor cell cultures would be useful in the treatment of graft-versus-host disease and the development of immune tolerance. Because control over stem and precursor cell differentiation can produce cell populations that are therapeutically useful, there is a need for the ability to control and regulate the differentiation of cells of myeloid dendritic cell lineage, or early progenitor cells, such as human CD34⁺ or CD133⁺ progenitor cells, for the controlled production of dendritic cells and/or granulocytes.

### 3. SUMMARY OF THE INVENTION

The present invention provides methods of modulating mammalian, particularly human stem cell or progenitor cell differentiation. In particular, the methods of the invention may be employed to regulate and control the differentiation and maturation of human stem cells along specific cell and tissue lineages. The invention encompasses the use of PDE IV inhibitors, particularly the class of compounds known as SelCIDS (Celgene), to effect such regulation and control. The invention further contemplated administration of these compounds to progenitor cells at specific times to modulate their differentiation in specific ways.

The methods of the invention encompass the regulation of differentiation of a stem cell or progenitor cell into a specific cell lineage, including, but not limited to, a mesenchymal, hematopoietic, adipogenic, hepatogenic, neurogenic, gliogenic, chondrogenic, vasogenic, myogenic, chondrogenic, or osteogenic lineage. In particular embodiment, the methods of the invention encompass the regulation of stem cell differentiation to a cell of a hematopoietic lineage.

The invention also encompasses the modulation of a committed cell to a specific cell type, e.g., mesenchymal cell, hematopoietic cell, adipocyte, hepatocyte, neuroblast, glioblast, chondrocyte, endothelial cell (EC) progenitor, myocyte, chondrocyte, or osteoblast. In specific embodiments, the invention encompasses the modulation of a committed hematopoietic progenitor cell to an erythrocyte, a thrombocyte, or a leukocyte (white blood cell) such as a neutrophil, monocyte, macrophage, eosinophil, basophil, mast cell, B-cell, T-cell, or plasma cell.

In another embodiment, the methods of the invention relate to modulating the differentiation of stem cells to cells of a hematopoietic lineage, in particular, CD34+, CD133+, and CD45+ hematopoietic lineages, and methods of producing prophylactically or therapeutically beneficial pharmaceutical compositions containing such cells. In another specific embodiment, the methods of the invention relate to modulating the differentiation of early progenitor cells into cells of a dendritic cell lineage or a granulocyte lineage, endothelial lineage, or cardiomyocyte lineage.

In another embodiment, the invention provides methods for regulating the differentiation of a progenitor cell into a hematopoietic lineage, particularly a dendritic cell or granulocytic lineage, endothelial lineage, neural lineage or cardiomyocyte lineage. In a specific embodiment, said progenitor cell is a CD34+ or CD133+ cell. Such regulation is accomplished by contacting the progenitor cells during culture with a compound of the invention. In one embodiment, said compound in an inhibitor of PDE IV activity. In a more specific embodiment, said compound is a PDE IV inhibitor. More preferably, said PDE IV inhibitor is a SelCID™ (see Section 4.3, below).

In another specific embodiment, the methods of the invention encompass the suppression of progenitor cell differentiation into a dendritic cell. In another specific embodiment, the invention provides a method for modulating the differentiation of progenitor cells during the first six days of culture to produce an expanded culture of such progenitor cells. In another embodiment, the methods of the invention encompass the promotion of early progenitor cell development into a granulocyte, which may be useful for fighting infections. The increase of granulocyte lineage committed progenitors (CD15⁺ cells) can be of potential use in the reduction of neutropenia and its subsequent infectious complications that represent the most common dose-limiting toxicity of cancer chemotherapy. In another embodiment, the methods of the invention may be used to suppress dendritic cell differentiation, which is useful for mitigating the effects of graft-versus-host disease.

The progenitor cells of the invention, as modulated by a compound of the invention, are useful for transplantation (i.e., hematopoietic reconstitution), and may be used in regenerative medicine as a renewable source of replacement cells and tissues (such as pancreatic, cardiac, hepatic, kidney, liver, brain, lung, bladder, intestinal or muscle cells) to treat normal senescence, injury or diseases such as heart disease, stroke, Parkinson's disease, and Alzheimer's disease. The cells will also be useful in the determination of the intracellular biochemical pathways that mediate the action of the compounds of the invention. These cells may also be useful for the screening of new drugs and toxins, for example, to determine potential anti-cancer drugs, to understand the origins of birth defects, etc.

The methods of the invention may be used to suppress specifically the generation of red blood cells or erythropoietic colonies (BFU-E and CFU-E), while augmenting both the generation of leukocyte and platelet forming colonies (CFU-GM) and enhancing total colony forming unit production. The methods of the invention may be used not only to regulate the differentiation of stem cells, and progenitor cells such as CD34+ progenitor cells, but may also be used to stimulate the rate of colony formation, providing significant benefits to hematopoietic stem cell transplantation by improving the speed of bone marrow engraftment.

Any mammalian stem cell can be used in accordance with the methods of the invention, including but not limited to, stem cells isolated from cord blood, placenta and other sources. The stem cells may be isolated from any mammalian species, e.g. mouse, rat, rabbit, guinea pig, dog, cat, pig, sheep, cow, horse, monkey, etc., more preferably, a human. The stem cells may include pluripotent cells, i.e., cells that have complete differentiation versatility, that are self-renewing, and can remain dormant or quiescent within tissue. The stem cells may also include multipotent cells or committed progenitor cells. In one preferred embodiment, the invention utilizes stem cells that are viable, quiescent, pluripotent stem cells that exist within, or are later produced by, the full-term placenta, that is, such cells can be recovered following successful birth and placental expulsion, exsanguination and perfusion of the placenta, resulting in the production and recovery of as many as one billion nucleated cells, which yield 50 to 100 million multipotent and pluripotent stem cells. Such cells are referred to herein as human placental stem cells or embryonic-like stem cells.

In one particular embodiment of the invention, cells, for example cells endogenous to bone marrow or to a postpartum perfused placenta, including, but not limited to, embryonic-like stem cells, progenitor cells such as CD34+ or CD133+ cells, pluripotent cells and multipotent cells, are exposed to the compounds of the invention and induced to differentiate. The endogenous cells may be propagated in vitro. In another embodiment, the endogenous cells may be collected from the placenta and culture medium and cultured in vitro under conditions appropriate, and for a time sufficient, to induce differentiation to the desired cell type or lineage.

In another embodiment of the invention, the stem or progenitor cells are derived from other sources such as cord blood, peripheral blood or adult blood, and are exposed to the compounds of the invention and induced to differentiate. In a preferred embodiment, the differentiation is conducted in vitro under conditions appropriate, and for a time sufficient, to induce differentiation into the desired lineage or cell type. The compounds of the invention are used in the differentiation/culture media by addition, in situ generation, or in any other manner that permits contact of the stem or progenitor cells with the compounds of the invention.

It has been discovered that the timing of the administration of the compounds of the invention have a profound impact upon the differentiation of CD34⁺ progenitor cells. Thus, in one embodiment of the invention, differentiation of CD34⁺ progenitor cells into dendritic cells is delayed or suppressed by a method comprising contacting the progenitor cell on the first day of culture with a compound of the invention. In another embodiment, the development of CD1a⁺ cells from CD34⁺ progenitor cells is reduced or prevented by a method comprising contacting said progenitor cells with a compound of the invention on the first day of culture. In another embodiment, the persistence of a CD1a⁺ cell population derived from CD34⁺ progenitor cells is increased by contacting said progenitor cells with a compound of the invention after culturing said progenitor cells for six days in the absence of said compound.

The present invention also encompasses methods of modulating the differentiation of early progenitor cells, such as human CD34⁺ and CD133⁺ cells, comprising contacting the progenitor cells at various times during the proliferative and differentiative phases with one or more of the compound(s) of the invention. Thus, in one embodiment, the invention encompasses a method of modulating the differentiation of the progenitor cells comprising contacting said cells with one or more compound(s) of the invention on the first day of culture only. In another embodiment, said cells are contacted with said compound(s) in one dose on any day between the first day and the twelfth day of culture. In another embodiment, said cells are contacted at least two times with said compound(s), on different days, between days 0-12, inclusive. In yet another embodiment, said cells are contacted with one or more compound(s) twice a day, once a day, or once every other day during the proliferative and/or differentiation phases. In another embodiment, said contacting is performed in vitro. In yet another embodiment, said contacting is performed in vivo in a subject. In a more specific embodiment, said subject is a human, a non-human mammal, an bird or a reptile.

In sum, exposure of endogenous or exogenous stem or progenitor cells which may be cultured in a postpartum perfused placenta, to compounds of the invention may occur while the cells are cultured in the placenta, or preferably, may occur in vitro after the cells have been recovered and removed from the placenta.

The invention encompasses the use of compounds that have PDE IV inhibitory activity as modulators of stem and/or progenitor cell development. In specific embodiments, the compounds are PDE IV inhibitors such as classes of compounds known as SelCIDs™ (Celgene Corp., Warren, N.J.).

The invention also encompasses the transplantation of pretreated stem or progenitor cells to treat or prevent disease. In one embodiment, a patient in need of transplantation is also administered a compound of the invention before, during and/or after transplantation.

The invention further encompasses the use of a progenitor cell or specific cell type produced from a method of the invention. In other words, the invention encompasses the use of leukocytes, granulocytes, or dendritic cells made from the differentiation of a hematopoietic progenitor wherever said differentiation of the progenitor as modulated or regulated using a compound of the invention.

In other embodiments, the invention encompasses the control or regulation of stem cells in vivo by the administration of both a stem cell and a small molecule compound of the invention to a patient in need thereof.

In one embodiment, the invention provides a pharmaceutical composition comprising CD34+ or CD133+ progenitor cells that have been contacted with a compound of the invention, particularly one that inhibits the activity of PDE IV, in the first six days of culture, under conditions that promote proliferation and differentiation of said progenitor cells, and a pharmaceutically-acceptable carrier. In a specific embodiment, the pharmaceutical composition includes cells that have been collected and cryopreserved after six days of culture. In another specific embodiment, the cells of the pharmaceutical composition are CD34⁺CD38^{-CD}34^{-or CD}34⁺CD38^{-CD}34⁺ cells. In another specific embodiment, the compound with which the cells are contacted is a PDE IV inhibitor of the invention. In another specific embodiment, the compound with which the cells are contacted is a SelCID™.

In another embodiment, the invention also provides for method of making a pharmaceutical composition, comprising contacting CD34+ or CD133+ progenitor cells with a compound that inhibits PDE IV activity, wherein said progenitor cells are cultured for six days in a culture medium under culture conditions that allow proliferation and differentiation of said progenitor cells; collecting said cells after six days of culture; and combining said cells with a pharmaceutically-acceptable carrier. In a specific embodiment of this method, said contacting is performed on the first day of culture. In another specific embodiment of this method, said contacting is performed at least twice during said six days of culture. In another specific embodiment, the compound with which the cells are contacted is a PDE IV inhibitor of the invention. In another specific embodiment, the compound with which the cells are contacted is a SelCID™. In yet another specific embodiment of this method, said progenitor cells have been isolated from other blood cells prior to said culturing. In another specific embodiment of this method, said culture medium additionally contains GM-CSF and TNF-α. In more specific embodiment of this method, said SelCID™ is present in a concentration of between 0.1 μM and 10.0 μM. In another more specific embodiment of this method, said SelCID™ is present at a concentration of 1.0 μM. In another specific embodiment of this method, said cells are cryopreserved after said collecting.

The invention further provides a method for expanding a progenitor cell population in a mammalian subject, comprising administering a therapeutically effective amount of CD34+ or CD133+ progenitor cells and one or more SelCIDs™ to said recipient mammalian subject. In specific embodiment of this method, said progenitor cells are differentiated in the recipient mammalian subject. In another specific embodiment of this method, said progenitor cells are administered to said subject in a cell preparation that is substantially free of red blood cells. In another specific embodiment of this method, said progenitor cells are administered to the recipient mammalian subject in a cell preparation that comprises bone marrow cells, placental cells, cord blood cells or PBMCs. In another specific embodiment of this method, said progenitor cells are administered to the recipient mammalian subject in conjunction with a carrier. In another specific embodiment of this method, said progenitor cell is a CD34+ CD133+ progenitor cell. In another specific embodiment of this method, the progenitor cells express incorporated genetic material of interest.

The present invention also provides the cells that are produced by the above methods that are useful as pharmaceutical compositions.

In yet other embodiments, the invention encompasses methods of conditioning stem cells or progenitor cells, for example, CD34+ progenitor cells, following cryopreservation and thawing, to counteract the deleterious effects of cryopreservation and exposure to cryopreservatives on the stem cells. In certain embodiments, the invention provides methods of conditioning stem cells following cryopreservation and thawing, to counteract the deleterious effects of exposure to cryopreservatives (e.g. DMSO) on the proliferative and migratory capacity of stem cells.

3.1. Definitions

As used herein, the term “bioreactor” refers to an ex vivo system for propagating cells, producing or expressing biological materials and growing or culturing cells tissues, organoids, viruses, proteins, polynucleotides and microorganisms.

As used herein, “DC cells” refers to dendritic cells.

As used herein, “early progenitor cell” means a CD34+ progenitor cell, a CD133⁺ progenitor cell, or the mammalian, avian or reptilian equivalent of either.

As used herein, the term “embryonic stem cell” refers to a cell that is derived from the inner cell mass of a blastocyst (e.g., a 4- to 5-day-old human embryo) and that is pluripotent.

As used herein, the term “embryonic-like stem cell” refers to a cell that is not derived from the inner cell mass of a blastocyst. As used herein, an “embryonic-like stem cell” may also be referred to as a “placental stem cell.” An embryonic-like stem cell is preferably pluripotent. However, the stem cells which may be obtained from the placenta include embryonic-like stem cells, multipotent cells, and committed progenitor cells. According to the methods of the invention, embryonic-like stem cells derived from the placenta may be collected from the isolated placenta once it has been exsanguinated and perfused for a period of time sufficient to remove residual cells. Preferably, the embryonic-like stem cells are human, though they may be derived from any mammal.

As used herein, the term “exsanguinated” or “exsanguination,” when used with respect to the placenta, refers to the removal and/or draining of substantially all cord blood from the placenta. In accordance with the present invention, exsanguination of the placenta can be achieved by, for example, but not by way of limitation, draining, gravity induced efflux, massaging, squeezing, pumping, etc. In a preferred embodiment, exsanguination of the placenta may further be achieved by perfusing, rinsing or flushing the placenta with a fluid that may or may not contain agents, such as anticoagulants, to aid in the exsanguination of the placenta.

As used herein, the term “perfuse” or “perfusion” refers to the act of pouring or passaging a fluid over or through an organ or tissue, preferably the passage of fluid through an organ or tissue with sufficient force or pressure to remove any residual cells, e.g., non-attached cells from the organ or tissue. As used herein, the term “perfusate” refers to the fluid collected following its passage through an organ or tissue. In a preferred embodiment, the perfasate contains one or more anticoagulants.

As used herein, the term “endogenous cell” refers to a “non-foreign” cell, i.e., a “self” or autologous cell, that is derived from the placenta.

As used herein the term “exogenous cell” refers to a “foreign” cell, i.e., a heterologous cell (i.e., a “non-self” cell derived from a source other than the placental donor) or autologous cell (i.e., a “self” cell derived from the placental donor) that is-derived from an organ or tissue other than the placenta.

As used herein, “PDE IV inhibitor” refers to the compounds disclosed in Section 4.3, below.

As used herein, the term “organoid” refers to an aggregation of one or more cell types assembled in superficial appearance or in actual structure as any organ or gland of a mammalian body, preferably the human body.

As used herein, the term “multipotent cell” refers to a cell that has the capacity to grow into any of subset of the mammalian body's approximately 260 cell types. Unlike a pluripotent cell, a multipotent cell does not have the capacity to form all off the cell types.

As used herein, the term “pluripotent cell” refers to a cell that has complete differentiation versatility, i.e., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), an embryonic stem cell cannot usually form a new blastocyst.

As used herein, the term “progenitor cell” refers to a cell that is committed to differentiate into a specific type of cell or to form a specific type of tissue.

As used herein, the term “stem cell” refers to a master cell that can reproduce indefinitely to form the specialized cells of tissues and organs. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells.

As used herein, the term “totipotent cell” refers to a cell that is able to form a complete embryo (e.g., a blastocyst).

### 4. DETAILED DESCRIPTION OF THE INVENTION

The present invention is based, in part, on the unexpected discovery that the exposure of stem cells or progenitor cells to the compounds of the invention results in a regulatable means of controlling the differentiation of stem or progenitor cells into specific populations of progenitor cells or differentiation of progenitor cells into specific cell types, such as dendritic cells, granulocytes, endothelial cells or neural cells. In particular, the exposure of stem or progenitor cells to the compounds of the invention results in the regulatable differentiation and expansion of specific populations of hematopoietic cells, including CD34+, CD38+ and CD133+ cells. Such regulation of differentiation is accomplished without significant loss of yield due to cell death or differentiation to undesired cell types or cell lineages; in other words, the compounds of the invention do not cause apoptosis of one or more cell populations. Further, the exposure of hematopoietic progenitor cells to the compounds of the invention results in regulatable differentiation and expansion of specific cell types.

Thus, the present invention provides methods of modulating human stem cell differentiation, specifically CD34⁺ hematopoietic progenitor cell, and CD133⁺ progenitor cell differentiation. In particular, the present invention provides methods that employ small organic molecules that inhibit PDE IV activity to modulate the differentiation of progenitor cell populations along specific cell and tissue lineages. Further, the invention encompasses methods of expanding early progenitor cells, such as human CD 133⁺ or CD34⁺, particularly CD34⁺ CD38⁻ cells, for transplantation into mammals, birds or reptiles, comprising exposing hematopoietic progenitor cells to a PDE IV inhibitor or antagonist, wherein the inhibitor or antagonist is a small molecule. The invention also provides methods of producing other cell types from these early progenitor cells, including, but not limited to, cells of the brain, kidney, intestinal tract and muscle. The compounds of the invention also act to suppress dendritic cell differentiation, and promote granulocytic cell differentiation, from early progenitor cells, such as human CD34⁺ progenitor cells.

Examples of the small molecule compounds that may be used in connection with the invention, include, but are not limited to, compounds that inhibit PDE IV activity. Compounds that may be used in the methods of the invention are described in detail in Section 4.3. In particularly preferred embodiments, the compounds are SelCIDs™(Celgene).

The methods of the invention encompass the regulation of differentiation of a stem or progenitor cell into a specific cell lineage, including, but not limited to, a mesenchymal, hematopoietic, adipogenic, hepatogenic, neurogenic, gliogenic, chondrogenic, vasogenic, myogenic, chondrogenic, or osteogenic lineage comprising incubating the stem or progenitor cell with a compound of the invention, preferably in vitro, for a sufficient period of time to result in the differentiation of the cell into a cell of a desired cell lineage. In a specific embodiment, differentiation of a stem or progenitor cell into a cell of the hematopoietic lineage is modulated. In particular, the methods of the invention may used to modulate the generation of blood cell colony generation from CD34⁺, CD133⁺, and CD45⁺ hematopoietic progenitor cells in a dose-responsive manner.

The methods of the invention also encompass the regulation of differentiation of a CD34⁺ progenitor cell into dendritic cells comprising incubating the progenitor cell with a compound of the invention, preferably in vitro, for a sufficient period of time to result in the differentiation of the cell into a cell of a desired cell lineage. In a specific embodiment, differentiation of a such a progenitor cell into a cell of the dendritic cell lineage is modulated through contacting said cell with a PDE IV inhibitor, particularly a SelCID™, or an analog or prodrug of such inhibitor or SelCID™. In another specific embodiment, the differentiation of a CD34⁺ progenitor cell is modulated to suppress differentiation along a myeloid lineage and encourage differentiation along a granulocytic lineage. In a more specific embodiment, differentiation of a CD34+ progenitor cell into a cell of a granulocytic cell lineage is modulated by a method comprising contacting a CD34⁺ progenitor cell with a compound of the invention on the first day said progenitor cells are cultured.

Any mammalian stem or progenitor cell can be used in accordance with the methods of the invention, including but not limited to, stem cells isolated from cord blood (“CB” cells), placenta and other sources. The stem cells may include pluripotent cells, i.e., cells that have complete differentiation versatility, that are self-renewing, and can remain dormant or quiescent within tissue. The stem cells may also include multipotent cells or committed progenitor cells. In one preferred embodiment, the invention utilizes stem cells that are viable, quiescent, pluripotent stem cells that exist within the full-term placenta can be recovered following successful birth and placental expulsion, exsanguination and perfusion resulting in the recovery of multipotent and pluripotent stem cells.

In another preferred embodiment, the progenitor cells are early progenitor cells, particularly CD34⁺ or CD133⁺ cells. Preferably, CD34⁺ or CD133⁺ progenitor cells are derived from human bone marrow, placenta, or cord blood. Equivalents of these cells from other mammals may also be used. In mouse, for example, Sca⁺ progenitor cells may be used in the methods of the invention. Equivalent early progenitor cells from birds or reptiles may also be used.

In a particular embodiment of the invention, cells endogenous to the placenta, or produced by a postpartum perfused placenta, including, but not limited to, embryonic-like stem cells, progenitor cells, pluripotent cells and multipotent cells, are exposed to the compounds of the invention and induced to differentiate while being cultured in an isolated and perfused placenta. The endogenous cells propagated in the postpartum perfused placental may be collected, and/or bioactive molecules recovered from the perfusate, culture medium or from the placenta cells themselves.

In another embodiment of the invention, stem or progenitor cells that are derived from sources other than postpartum placenta are exposed to the compounds of the invention and induced to differentiate while being cultured in vitro. Thus, the invention encompasses methods for differentiating mammalian stem cells into specific progenitor cells comprising differentiating the stem cells under conditions and/or media suitable for the desired differentiation and in the presence of a compound of the invention.

Further, the invention encompasses methods for modulating or regulating the differentiation of a population of a specific progenitor cell into specific cell types comprising differentiating said progenitor cell under conditions suitable for said differentiation and in the presence of one or more compounds of the invention. Alternatively, the stem or progenitor cell can be exposed to a compound of the invention and subsequently differentiated using suitable conditions. Examples of suitable conditions include nutrient media formulations supplemented with human serum and cell culture matrices, such as MATRIGEL® supplemented with growth factors.

The method of the invention also contemplates that different cell populations may be produced by contacting the progenitor cell(s) with a compound of the invention at various times during culture, either at the proliferation or differentiation stage. See Section4.4, particularly Section 4.4.2, below.

In a specific embodiment, the present invention provides methods that employ small molecules, particularly PDE IV inhibitors, preferably SelCIDs or prodrugs thereof, to modulate and regulate hematopoiesis in the context of pre-transplantation conditioning of hematopoietic progenitors.

The present invention also provides methods that employ the small molecules of the invention to modulate and regulate hematopoiesis in the context of ex vivo conditioning of hematopoietic progenitors. The methods of the invention encompass the regulation of stem or progenitor cell differentiation in vitro, comprising incubating the stem or progenitor cells with the compound in vitro, followed by direct transplantation of the differentiated cells to a subject.

The invention also encompasses the control or regulation of stem or progenitor cells in vivo by the administration of both a stem or progenitor cell and a compound of the invention to a patient in need thereof.

The invention further encompasses the transplantation of pretreated stem or progenitor cells to treat or prevent disease. In one embodiment, a patient in need of transplantation is also administered a compound of the invention before, during and/or after transplantation. In another embodiment, a patient in need of transplantation is also administered untreated stem or progenitor cells, e.g., cord blood cells, adult blood cells, peripheral blood cells, or bone marrow cells. In another embodiment, the methods of the invention include the administration of the compounds to a subject that is the recipient of unconditioned stem cells or progenitor cells for the purpose of eliciting a modulatory effect on the stem cells that have already been transplanted.

In certain embodiments, the invention encompasses bone marrow transplantation which comprises transplanting cord blood (or stem cells obtained from cord blood), peripheral (i.e., adult) blood (or stem cells obtained from peripheral blood), wherein said cord blood or stem cells have been pretreated with a compound of the invention. Further, the invention encompasses the use of white blood cells made from hematopoietic progenitor cells that have been differentiated in the presence of a compound of the invention. For example, white blood cells produced by differentiating hematopoietic progenitor can be used in transplantation or can be mixed with cord blood or cord blood stem cells prior to transplantation.

In other embodiments, the invention encompasses bone marrow transplantation which comprises transplanting early progenitor cells, such as CD34⁺ or CD133⁺ progenitor cells, obtained according to the methods of the invention, wherein said progenitor cells have been pretreated with a compound of the invention. In one embodiment of the invention, said dendritic cell precursors are CD34⁺ CD38⁻CD33⁺ or CD34⁺ CD38^{-CD}33− precursor cells. Further, the invention encompasses the use of cells made from CD34⁺ progenitor cells that have been differentiated in the presence of a compound of the invention. For example, CD34⁺ CD38^{-CD}33⁺ precursor cells, CD34⁺ CD38^{-CD}33⁻ precursor cells, granulocytes, etc. produced by the differentiation of CD34⁺ progenitor cells using the compounds of the invention can be used in transplantation. Cells differentiated from CD133⁺ cells, using the compounds of the invention, are also encompassed by the present invention.

The invention further encompasses methods of conditioning stem cells following cryopreservation and thawing, to counteract the deleterious effects of cryopreservation and exposure to cryopreservatives on the stem cells. In certain embodiments, the invention provides methods of conditioning stem cells following cryopreservation and thawing, to counteract the deleterious effects of exposure to cryopreservatives (e.g. DMSO) on the proliferative and migratory capacity of stem cells.

4.1. Modulation of Differentiation of Stem Cells and CD34⁺ or CD133⁺ Progenitor Cells

4.1.1. Stem Cells

The present invention provides methods of modulating human stem cell differentiation. In certain embodiments, the methods of the invention encompass the regulation of stem or progenitor cell differentiation in vitro, comprising incubating the stem cells with the compound in vitro, followed by direct transplantation of the differentiated cells to a subject. In other embodiments, the methods of the invention encompass the regulation of stem or progenitor cell differentiation in vivo, comprising delivering the compounds to a subject that is the recipient of unconditioned stem cells, followed by direct administration of the compound to the subject.

The embryonic-like stem cells obtained by the methods of the invention may be induced to differentiate along specific cell lineages, including, but not limited to a mesenchymal, hematopoietic, adipogenic, hepatogenic, neurogenic, gliogenic, chondrogenic, vasogenic, myogenic, chondrogenic, or osteogenic lineage.

In certain embodiments, embryonic-like stem cells obtained according to the methods of the invention are induced to differentiate for use in transplantation and ex vivo treatment protocols. In certain embodiments, embryonic-like stem cells obtained by the methods of the invention are induced to differentiate into a particular cell type and genetically engineered to provide a therapeutic gene product. In a specific embodiment, embryonic-like stem cells obtained by the methods of the invention are incubated with a compound, such as a small organic molecule, in vitro, that induces it to differentiate, followed by direct transplantation of the differentiated cells to a subject. In a preferred embodiment, the compounds that are used to control or regulate differentiation of stem cells are not polypeptides, peptides, proteins, hormones, cytokines, oligonucleotides or nucleic acids.

Stem cells that may be used in accordance with the invention include, but are not limited to, cord blood (CB) cells, placental cells, embryonic stem (ES) cells, embryonic-like stem cells, trophoblast stem cells, progenitor cells, bone marrow stem cells and multipotent, pluripotent and totipotent cells.

In particular, the methods of the invention encompass the regulation of the differentiation of stem cell populations, in addition to mesenchymal stem cells, into specific tissue lineages. For example, the methods of the invention may be employed to regulate the differentiation of a multipotent stem cell into chondrogenic, vasogenic, myogenic, and osteogenic lineage cells by promoting specific musculoskeletal regeneration and repair, neoangiogenesis, and repopulation of specific muscular tissues, such as myocardium and skeletal muscle, and revascularization of a variety of organs and tissues including, but not limited to brain, spinal cord, liver, lung, kidney and pancreas. The methods of the invention may be employed to regulate differentiation of a multipotent stem cell into cell of adipogenic, chondrogenic, osteogenic, neurogenic or hepatogenic lineage.

The agent used to modulate differentiation can be introduced into the postpartum perfused placenta to induce differentiation of the cells being cultured in the placenta. Alternatively, the agent can be used to modulate differentiation in vitro after the cells have been collected or removed from the placenta.

The methods of the invention encompass the regulation of progenitor stem cell differentiation to a cell of the hematopoietic lineage, comprising incubating the progenitor stem cells with the compound in vitro for a sufficient period of time to result in the differentiation of these cells to a hematopoietic lineage. In particular, the methods of the invention may used to modulate the generation of blood cell colony generation from CD34+, CD133+, and CD45+ hematopoietic progenitor cells in a dose-responsive manner (for discussion of dosing, see Section 4.7).

Preferably, the methods of the invention may be used to suppress specifically the generation of red blood cells or erythropoietic colonies (BFU-E and CFU-E), while augmenting both the generation of leukocyte and platelet forming colonies (CFU-GM) and enhancing total colony forming unit production. The methods of the invention may be used not only to regulate the differentiation of stem cells, but may also be used to stimulate the rate of colony formation, providing significant benefits to hematopoietic stem cell transplantation by improving the speed of bone marrow engraftment and recovery of leukocyte and/or platelet production.

In other embodiments, the methods of the invention may be used to regulate the differentiation of e.g., a neuronal precursor cell or neuroblast into a specific neuronal cell type such as a sensory neuron (e.g., a retinal cell, an olfactory cell, a mechanosensory neuron, a chemosensory neuron, etc.), a motor neuron, a cortical neuron, or an interneuron. In other embodiments, the methods of the invention may be used to regulate the differentiation of cell types including, but not limited to, cholinergic neurons, dopaminergic neurons, GABA-ergic neurons, glial cells (including oligodendrocytes, which produce myelin), and ependymal cells (which line the brains ventricular system). In yet other embodiments, the methods of the invention may be used to regulate the differentiation of cells that are constituent of organs, including, but not limited to, purkinje cells of the heart, biliary epithelium of the liver, beta-islet cells of the pancreas, renal cortical or medullary cells, and retinal photoreceptor cells of the eye.

Assessment of the differentiation state of stem cells obtained according to the methods of the invention may be identified by the presence of cell surface markers. Embryonic-like stem cells of the invention, for example, may be distinguished by the following cell surface markers: OCT-4+ and ABC-pt. Further, the invention encompasses embryonic-like stem cells having the following markers: CD10, CD29, CD44, CD54, CD90, SH2, SH3, SH4, OCT-4 and ABC-p, or lacking the following cell surface markers: CD34, CD38, CD45, SSEA3 and SSEA4, as described hereinabove. Such cell surface markers are routinely determined according to methods well known in the art, e.g. by flow cytometry, followed by washing and staining with an anti-cell surface marker antibody. For example, to determine the presence of CD34 or CD38, cells maybe washed in PBS and then double-stained with anti-CD34 phycoerythrin and anti-CD38 fluorescein isothiocyanate (Becton Dickinson, Mountain View, Calif.).

4.1.2. CD34+ And CD133+ Early Progenitor Cells

The present invention also provides methods of modulating human CD34⁺ or CD133⁺ cell differentiation. In certain embodiments, the methods of the invention encompass the regulation of stem or progenitor cell differentiation in vitro, comprising incubating the stem cells with the compound in vitro, followed by direct transplantation of the differentiated cells to a subject.

The progenitor cells obtained by the methods of the invention may be induced to differentiate along specific cell lineages, including, but not limited to, for CD34⁺ progenitor cells, a myeloid or granulocytic, lineage, and for CD 133⁺ cells, an endothelial or neural cell lineage. In certain embodiments, progenitor cells are induced to differentiate for use in transplantation and ex vivo treatment protocols. In certain embodiments, progenitor cells are induced to differentiate into a particular cell type and genetically engineered to provide a therapeutic gene product. In a specific embodiment, progenitor cells are incubated with a compound, such as a small organic molecule, in vitro, that induces it to differentiate, followed by direct transplantation of the differentiated cells to a subject. In a preferred embodiment, the compounds that are used to control or regulate differentiation of stem cells are not polypeptides, peptides, proteins, hormones, cytokines, oligonucleotides or nucleic acids. In another preferred embodiment, the progenitor cell is caused to differentiate into a CD34^{+CD}38^{-CD}33^{+ or CD}34^{+CD}38⁻CD33⁻ progenitor cell.

Preferably, the methods of the invention may be used to suppress specifically the generation of red blood cells or erythropoietic colonies (BFU-E and CFU-E), while augmenting both the generation of leukocyte and platelet forming colonies (CFU-GM) and enhancing total colony forming unit production. The methods of the invention may be used not only to regulate the differentiation of stem cells, but may also be used to stimulate the rate of colony formation, providing significant benefits to hematopoietic stem cell transplantation by improving the speed of bone marrow engraftment and recovery of leukocyte and/or platelet production.

In other embodiments, the methods of the invention may be used to reduce the differentiation of CD34⁺ progenitor cells into CD1a⁺ cells, particularly CD86⁺CD1a⁺ cells. In another embodiment, the methods of the invention may be used to reduce or prevent the differentiation of CD34⁺ progenitor cells into CD14⁺CD1a− cells. CD14⁺CD1a− cells are dermal dendritic cell or monocyte/macrophage progenitor cells. In another embodiment, the methods of the invention may be used to reduce the expression on proliferating CD34⁺ progenitor cells of co-stimulatory molecules CD80 and CD86. In another embodiment, the methods of the invention may be used to reduce the differentiation of proliferating CD34⁺ progenitor cells into CD54^{bright} cells, and to encourage differentiation into CD54^{dim} cells. In another embodiment, the methods of the invention may be used to increase the number of CD133⁺ cells, which are endothelial cell progenitor cells. In yet another embodiment, the methods of the invention may be used to decrease the differentiation of proliferating CD34+cells into CD11c−CD 15⁺ cells, and increase differentiation into CD11c⁺CD15− cells, thus shifting differentiation from a myeloid dendritic cell lineage to a granulocytic lineage.

Assessment of the differentiation state of stem cells obtained according to the methods of the invention may be identified by the presence of cell surface markers. Progenitor cells of the invention, for example, may be distinguished by the CD34⁺ or CD133⁺ cell surface markers. Further, the invention encompasses proliferating progenitor cells possessing, or showing increased expression relative to a control, of one or more of the following markers: CD15, CD34, CD33, CD133, or CD54^{dim}, as described hereinabove. The invention further encompasses proliferating progenitor cells lacking, or showing reduced expression relative to a control, of one of more of the following markers: HLA-DR, CD1a, CD11c, CD38, CD80, CD86, CD54^{bright} or CD14. In a preferred embodiment, proliferating progenitor cells of the invention exhibit CD34⁺CD38^{-CD}33⁺ or CD34⁺CD38⁻CD33⁻. Such cell surface markers are routinely determined according to methods well known in the art, e.g. by washing and staining with an anti-cell surface marker antibody, followed by flow cytometry. For example, to determine the presence of CD34 or CD38, cells may be washed in PBS and then double-stained with anti-CD34 phycoerythrin and anti-CD38 fluorescein isothiocyanate (Becton Dickinson, Mountain View, Calif.).

In certain embodiment, differentiated cells may be characterized by characterizing the phagocytic capacity of the differentiated cells. The capacity of differentiated, or differentiating, cells to phagocytose may be assessed by, for example, labeling dextran with FITC and determining the amount of uptake by known methods. The capacity of differentiated, or differentiating, cells to ability to stimulate T cells may be assessed in a mixed leukocyte reaction (MLR), in which presumptively antigen-loaded cells are mixed with T cells, and the level of T cell activation is determined.

4.1.3. Identification and Characterization of Cells

In certain embodiments, differentiated cells maybe identified by characterizing differentially expressed genes (for example, characterizing a pool of genes from an undifferentiated progenitor cell(s) of interest versus a pool of genes from a differentiated cell derived from the progenitor cell). For example, nucleic acid amplification methods such as polymerase chain reaction (PCR) or transcription-based amplification methods (e.g., in vitro transcription (IVT)) may be used to profile gene expression in different populations of cells, e.g., by use of a polynucleotide microarray. Such methods to profile differential gene expression are well known in the art (see; e.g., Wieland et al., Proc. Natl. Acad: Sci. USA 87: 2720-2724 (1990; Lisitsyn et al., Science 259: 946951 (1993); Lisitsyn et al., Meth. Enzymology 254: 291-304 (1995); U.S. Pat. No. 5,436,142; U.S. Pat; No. 5,501.,964; Lisitsyn et al., Nature Genetics 6: 57-63 (1994); Hubank and Schatz, 1994, Nucleic Acids Research 22: 5640-5648; Zeng et al., 1994, Nucleic Acids Research 22: 4381-4385; U.S. Pat. No. 5,525,471; Linsley et al., U.S. Pat. No. 6,271,002, entitled “RNA amplification method,” issued Aug. 7, 2001; Van Gelder et al., U.S. Pat. No. 5,716,785, entitled “Processes for genetic manipulations using promoters,” issued Feb. 10, 1998; Stoflet et al., 1988, Science 239:491-494, 1988; Sarkar and Sommer, 1989, Science 244: 331-334; Mullis et al., U.S. Pat. No. 4,683,195; Malek et al., U.S. Pat. No. 5,130,238; Kacian and Fultz, U.S. Pat. No. 5,399,491; Burg et al., U.S. Pat. No. 5,437,990; R. N Van Gelder et al. (1990), Proc. Natl. Acad. Sci. USA 87, 1663; D. J. Lockhart et al., 1996, Nature Biotechnol. 14, 1675; Shannon, U.S. Pat. No. 6,132,997; Lindemann et al., U.S. Pat. No. 6,235,503, entitled “Procedure for subtractive hybridization and difference analysis,” issued May 22, 2001).

Commercially available kits are available for gene profiling, e.g., the displayPROFILE™ series of kits (Qbiogene, Carlsbad, Calif., which uses a gel-based approach for profiling gene expression. The kits utilize Restriction Fragment Differential Display-PCR(RFDD-PCR) to compare gene expression patterns in eukaryotic cells. A PCR-Select Subtraction Kit (Clontech) and a PCR-Select Differential Screening Kit (Clontech) may also be used, which permits identification of differentially expressed clones in a subtracted library. After generating pools of differentially expressed genes with the PCR-Select Subtraction kit, the PCR-Select Differential Screening kit is used. The subtracted library is hybridized with probes synthesized directly from tester and driver populations, a probe made from the subtracted cDNA, and a probe made from reverse-subtracted cDNA (a second subtraction performed in reverse). Clones that hybridize to tester but not driver probes are differentially expressed; however, non-subtracted probes are not sensitive enough to detect rare messages. Subtracted probes are greatly enriched for differentially expressed cDNAs, but may give false positive results. Using both subtracted and non-subtracted probes according to the manufacturer's (Clontech) instructions identifies differentially expressed genes.

In another embodiment, differentiated stem or progenitor cells are identified and characterized by a colony forming unit assay, which is commonly known in the art, such as Mesen Cult™ medium (Stem Cell Technologies, Inc., Vancouver British Columbia).

Determination that a stem cell or progenitor has differentiated into a particular cell type may be accomplished by methods well-known in the art, e.g. measuring changes in morphology and cell surface markers using techniques such as flow cytometry or immunocytochemistry (e.g., staining cells with tissue-specific or cell-marker specific antibodies), by examination of the morphology of cells using light or confocal microscopy, or by measuring changes in gene expression using techniques well known in the art, such as PCR and gene-expression profiling.

4.2. Stem and Progenitor Cell Populations

The present invention provides methods of modulating human stem cell differentiation. Any mammalian stem cell can be used within the methods of the invention, including, but not limited to, stem cells isolated from cord blood (CB cells), peripheral blood, adult blood, bone marrow, placenta, mesenchymal stem cells and other sources. In a non-preferred embodiment, the stem cells are embryonic stem cells or cells that have been isolated from sources other than placenta.

Sources of mesenchymal stem cells include bone marrow, embryonic yolk sac, placenta, umbilical cord, fetal and adolescent skin, and blood. Bone marrow cells may be obtained, for example, from iliac crest, femora, tibiae, spine, rib or other medullary spaces.

The stem cells to be used in accordance with the methods of the present invention may include pluripotent cells, i.e., cells that have complete differentiation versatility, that are self-renewing, and can remain dormant or quiescent within tissue. The stem cells may also include multipotent cells, committed progenitor cells, and fibroblastoid cells. In one preferred embodiment, the invention utilizes stem cells that are viable, quiescent, pluripotent stem cells isolated from a full-term exsanguinated perfused placenta.

Stem cell populations may consist of placental stem cells obtained through a commercial service, e.g. LifeBank USA (Cedar Knolls, N.J.), ViaCord (Boston Mass.), Cord Blood Registry (San Bruno, Calif.) and Cryocell (Clearwater, Fla.).

Stem cell populations may also consist of placental stem cells collected according to the methods disclosed in U.S. Application Publication No. U.S. 20020123141, published Sep. 5, 2002, entitled “Method of Collecting Placental Stem Cells” and U.S. Application Publication No. U.S. 20030032179, published Feb. 13, 2003, entitled “Post-Partum Mammalian Placenta, Its Use and Placental Stem Cells Therefrom” (both of which are incorporated herein by reference in their entireties).

In one embodiment, stem cells from cord blood may be used. The first collection of blood from the placenta is referred to as cord blood, which contains predominantly CD34⁺ and CD38⁺ hematopoietic progenitor cells. Within the first twenty-four hours of postpartum perfusion, high concentrations of CD34⁺CD38⁻ hematopoietic progenitor cells may be isolated from the placenta. After about twenty-four hours of perfusion, high concentrations of CD34⁻CD38⁻ cells can be isolated from the placenta along with the aforementioned cells. The isolated perfused placenta of the invention provides a source of large quantities of stem, cells enriched for CD34⁺CD38⁻ stem cells and CD34⁻CD38⁺ stem cells: The isolated placenta that has been perfused for twenty-four hours or more provides a source of large quantities of stem cells enriched for CD34⁻ and CD38⁻ stem cells.

Preferred cells to be used in accordance with the present invention are embryonic-like stem cells that originate from an exsanguinated perfused placenta, or cells that derive from embryonic-like placental stem cells. The embryonic-like stem cells off the invention may be characterized by measuring changes in morphology and cell surface markers using techniques such as flow cytometry and immunocytochemistry, and measuring changes in gene expression using techniques, such as PCR In one embodiment of the invention, such embryonic-like stem cells may be characterized by the presence of the following cell surface markers: CD10, CD29, CD44, CD54, CD90, SH2, SH3, SH4, OCT-4 and ABC-p, or the absence of the following cell surface markers: CD34, CD38, CD45, SSEA3 and SSEA4. In a preferred embodiment, such embryonic-like stem cells may be characterized by the presence of cell surface markers OCT-4 and APC-p. Such cell surface markers are routinely determined according to methods well known in the art, e.g. by flow cytometry, followed by washing and staining with an anti-cell surface marker antibody. For example, to determine the presence of CD34 or CD38, cells may be washed in PBS and then double-stained with anti-CD34 phycoerythrin and anti-CD38 fluorescein isothiocyanate (Becton Dickinson, Mountain View, Calif.).

Embryonic-like stem cells originating from placenta have characteristics of embryonic stem cells but are not derived from the embryo. In other words, the invention encompasses the use of OCT-4+ and ABC-p+cells that are undifferentiated stem cells that are isolated from a postpartum perfused placenta. Such cells are as versatile (e.g., pluripotent) as human embryonic stem cells. As mentioned above, a number of different pluripotent or multipotent stem cells can be isolated from the perfused placenta at different time points e.g., CD34+ CD38+, CD34+ CD38−, and CD34−CD38− hematopoietic cells. According to the methods of the invention, human placenta is used post-birth as the source of embryonic-like stem cells.

For example, after expulsion from the womb, the placenta is exsanguinated as quickly as possible to prevent or minimize apoptosis. Subsequently, as soon as possible after exsanguination the placenta is perfused to remove blood, residual cells, proteins, factors and any other materials present in the organ. Materials debris may also be removed from the placenta. Perfusion is normally continued with an appropriate perfusate for at least two to more than twenty-four hours. In several additional embodiments the placenta is perfused for at least 4, 6, 8, 10, 12, 14, 16, 18, 20, and 22 hours. In other words, this invention is based at least in part on the discovery that the cells of a postpartum placenta can be activated by exsanguination and perfusion for a sufficient amount of time. Therefore, the placenta can readily be used as a rich and abundant source of embryonic-like stem cells, which cells can be used for research, including drug discovery, treatment and prevention of diseases, in particular transplantation surgeries or therapies, and the generation of committed cells, tissues and organoids. See co-pending application Ser. No. 10/004,942, filed Dec. 5, 2001 entitled “Method of Collecting Placental Stem Cells” and application Ser. No. 10/076,180, filed Feb. 13, 2002, entitled “Post-Partum Mammalian Placenta, Its Use and Placental Stem Cells Therefrom,” both of which are incorporated herein by reference in their entireties.

Embryonic-like stem cells are extracted from a drained placenta by means of a perfusion technique that utilizes either or both of the umbilical artery and umbilical vein. The placenta is preferably drained by exsanguination and collection of residual blood (e.g., residual umbilical cord blood). The drained placenta is then processed in such a manner as to establish an ex vivo, natural bioreactor environment in which resident embryonic-like stem cells within the parenchyma and extravascular space are recruited. The embryonic-like stem cells migrate into the drained, empty microcirculation where, according to the methods of the invention, they are collected, preferably by washing into a collecting vessel by perfusion.

Specifically contemplated as part of the invention is the modulation of CD34⁺ and CD133⁺ progenitor cells into myeloid cells, particularly dendritic or granulocytic cells. Recent reports indicate that such cells are pluripotent; thus, the invention also contemplates the modulation of the development of these progenitor into cells of the brain, kidney, intestinal tract, liver or muscle.

Any mammalian, avian or reptilian CD34⁺ or CD133⁺ stem or progenitor cell can be used within the methods of the invention, including, but not limited to, stem cells isolated from cord blood (CB cells), peripheral blood, adult blood, bone marrow, placenta, including perfused placenta (see U.S. Application Publication No. U.S. 20030032179, published Feb. 13, 2003, entitled “Post-Partum Mammalian Placenta, Its Use and Placental Stem Cells Therefrom”, which is incorporated herein by reference in its entirety), mesenchymal stem cells and other sources. In a preferred embodiment, the stem cells are hematopoietic stem cells or cells that have been isolated from bone marrow. Such cells may be obtained from other organs or tissues, but such sources are less preferred.

In one embodiment, progenitor cells from cord blood or from post-partum placenta may be used. As noted above, cord blood contains predominantly CD34+ and CD38+ hematopoietic progenitor cells. Within the first twenty-four hours of postpartum perfusion, high concentrations of CD34+ CD38− hematopoietic progenitor cells may be isolated from an isolated, perfused placenta. After about twenty-four hours of perfusion, high concentrations of CD34− CD38− cells can be isolated from the placenta along with the aforementioned cells. In another embodiment, progenitor cell populations may be obtained through a commercial service, e.g., LifeBank USA (Cedar Knolls, N.J.), ViaCord (Boston Mass.), Cord Blood Registry (San Bruno, Calif.) and Cryocell (Clearwater, Fla.).

4.3. The Compounds of the Invention

Compounds used in the invention include racemic, stereomerically pure or stereomerically enriched selective cytokine inhibitory drugs, stereomerically or enantiomerically pure compounds that have selective cytokine inhibitory activities, and pharmaceutically acceptable salts, solvates, hydrates, stereoisomers, clathrates, and prodrugs thereof. Preferred compounds used in the invention are known Selective Cytokine Inhibitory Drugs (SelCIDs™) of Celgene Corporation.

As used herein and unless otherwise indicated, the term “SelCIDs™” used in the invention encompasses small molecule drugs, e.g., small organic molecules which are not peptides, proteins, nucleic acids, oligosaccharides or other macromolecules. Preferred compounds inhibit TNF-α production. Further, the compounds may also have a modest inhibitory effect on LPS induced IL-1β and IL-12. More preferably, the compounds of the invention are potent PDE4 inhibitors. PDE4 is one of the major phosphodiesterase isoenzymes found in human myeloid and lymphoid lineage cells. The enzyme plays a crucial part in regulating cellular activity by degrading the ubiquitous second messenger cAMP and maintaining it at low intracellular levels. Without being limited by theory, inhibition of PDE4 activity results in increased cAMP levels leading to the modulation of LPS induced cytokines, including inhibition of TNF-α production in monocytes as well as in lymphocytes.

Specific examples of selective cytokine inhibitory drugs include, but are not limited to, the cyclic imides disclosed in U.S. Pat. No. 5,605,914; the cycloalkyl amides and cycloalkyl nitrites of U.S. Pat. Nos. 5,728,844 and 5,728,845, respectively; the aryl amides (for example, an embodiment being N-benzoyl-3-amino-3-(3′,4′-dimethoxyphenyl)-propanamide) of U.S. Pat. Nos. 5,801,195 and 5,736,570; the imide/amide ethers and alcohols (for example 3-phthalimido-3-(3′,4′-dimethoxypheryl)propan-1-ol) disclosed in U.S. Pat. No. 5,703,098; the succinimides and maleimides (for example methyl 3-(3′,4′,5′6′-petrahydrophthalimdo)-3-(3″,4″-dimethoxyphenyl)propionate) disclosed in U.S. Pat. No. 5,658,940; imido and amido substituted alkanohydroxamic acids disclosed in WO 99/06041 and substituted phenethylsulfones disclosed in U.S. Pat. No. 6,020,358; and aryl amides such as N-benzoyl-3-amino-3-(3′,4′dimethoxyphenyl)propanamide as described in U.S. Pat. No. 6,046,221. The entireties of each of the patents and patent applications identified herein are incorporated herein by reference.

Additional selective cytokine inhibitory drugs belong to a family of synthesized chemical compounds of which typical embodiments include 3-(1,3-dioxobenzo-[f]isoindol-2-yl)-3-(3-cyclopentyloxy-4-methoxyphenyl)propionamide and 3-(1,3-dioxo-4-azaisoindol-2-yl)-3-(3,4-dimethoxyphenyl)-propionamide.

Other specific selective cytokine inhibitory drugs belong to a class of non-polypeptide cyclic amides disclosed in U.S. Pat. Nos. 5,698,579 and 5,877,200, both of which are incorporated herein. Representative cyclic amides include compounds of the formula: 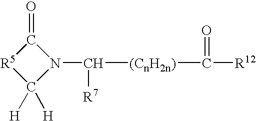
wherein n has a value of 1, 2, or 3;
R⁵ is o-phenylene, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkyl of 1 to 10 carbon atoms, and halo;
R⁷ is (i) phenyl or phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (ii) benzyl unsubstituted or substituted with 1 to 3 substituents selected from the group consisting of nitro, cyano, trifluoromethyl, carbothoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, (iii) naphthyl, and (iv) benzyloxy;
   R¹² is —OH, alkoxy of 1 to 12 carbon atoms, or 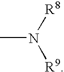
R⁸ is hydrogen or alkyl of 1 to 10 carbon atoms; and
R⁹ is hydrogen, alkyl of 1 to 10 carbon atoms, —COR¹⁰, or —SO₂R¹⁰, wherein R¹⁰ is hydrogen, alkyl of 1 to 10 carbon atoms, or phenyl.

Specific compounds of this class include, but are not limited to:
3-phenyl-2-(1-oxoisoindolin-2-yl)propionic acid;
3-phenyl-2-(1-oxoisoindolin-2-yl)propionamide;
3-phenyl-3-(1-oxoisoindolin-2-yl)propionic acid;
3-phenyl-3-(1-oxoisoindolin-2-yl)propionamide;
3-(4-methoxyphenyl)-3-(1-oxisoindolin-yl)propionic acid;
3-(4-methoxyphenyl)-3-(1-oxisoindolin-yl)propionamide;
3-(3,4-dimethoxyphenyl)-3-(1-oxisoindolin-2-yl)propionic acid;
3-(3,4-dimethoxy-phenyl)-3-(1-oxo-1,3-dihydroisoindol-2-yl)-propionamide;
3-(3,4-dimethoxyphenyl)-3-(1-oxisoindolin-2-yl)propionamide;
3-(3,4-diethoxyphenyl)-3-(1-oxoisoindolin-yl)propionic acid;
methyl 3-(1-oxoisoindolin-2-yl)-3-(3-ethoxy-4-methoxyphenyl)propionate;
3-(1-oxoisoindolin-2-yl)-3-(3-ethoxy4-methoxyphenyl)propionic acid;
3-(1-oxoisoindolin-2-yl)-3-(3-propoxy-4-methoxyphenyl)propionic acid;
3-(1-oxoisoindolin-2-yl)-3-(3-butoxy-4-methoxyphenyl)propionic acid;
3-(1-oxoisoindolin-2-yl)-3-(3-propoxy-4-methoxyphenyl)propionamide;
3-(1-oxoisoindolin-2-yl)-3-(3-butoxy-4-methoxyphenyl)propionamide;
methyl 3-(1-oxoisoindolin-2-yl)-3-(3-butoxy-4-methoxyphenyl)propionate; and
methyl 3-(1-oxoisoindolin-2-yl)-3-(3-propoxy-4-methoxyphenyl)propionate.

Other specific selective cytokine inhibitory drugs include the imido and amido substituted alkanohydroxamic acids disclosed in WO 99/06041, which is incorporated herein by reference. Examples of such compound include, but are not limited to: 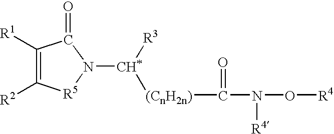
wherein each of R¹ and R², when taken independently of each other, is hydrogen, lower alkyl, or R¹ and R², when taken together with the depicted carbon atoms to which each is bound, is o-phenylene, o-naphthylene, or cyclohexene-1,2-diyl, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, and halo;
R³ is phenyl substituted with from one to four substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, alkylthio of 1 to 10 carbon atoms, benzyloxy, cycloalkoxy of 3 to 6 carbon atoms, C₄-C₆-cycloalkylidenemethyl, C₃-C₁₀-alkylidenemethyl, indanyloxy, and halo;
R⁴ is hydrogen, alkyl of 1 to 6 carbon atoms, phenyl, or benzyl;
R^{4′} is hydrogen or alkyl of 1 to 6 carbon atoms;
R⁵ is —CH₂—, —CH₂—CO—, —SO₂—, —S—, or —NHCO—;
n has a value of 0, 1, or 2; and
the acid addition salts of said compounds which contain a nitrogen atom capable of being protonated.

Additional specific selective cytokine inhibitory drugs used in the invention include, but are not limited to:
3-(3-ethoxy-4-methoxyphenyl)-N-hydroxy-3-(1-oxoisoindolinyl)propionamide;
3-(3-ethoxy-4-methoxyphenyl)-N-methoxy-3-(1-oxoisoindolinyl)propionamide;
N-benzyloxy-3-(3-ethoxy-4-methoxyphenyl)-3-phthalimidopropionamide;
N-benzyloxy-3-(3-ethoxy-4-methoxyphenyl)-3-(3-nitrophthalimido)propionamide;
N-benzyloxy-3-(3-ethoxy-4-methoxyphenyl)-3-(1-oxoisoindolinyl)propionamide;
3-(3-ethoxy-4-methoxyphenyl)-N-hydroxy-3-phthalimidopropionamide;
N-hydroxy-3-(3,4-dimethoxyphenyl)-3-phthalimidopropionamide;
3-(3-ethoxy-4-methoxyphenyl)-N-hydroxy-3-(3-nitrophthalimido)propionamide;
N-hydroxy-3-(3,4-dimethoxyphenyl)-3-(1-oxoisoindolinyl)propionamide;
3-(3-ethoxy-4-methoxyphenyl)-N-hydroxy-3-(4-methyl-phthalimido)propionamide;
3-(3-cyclopentyloxy-4-methoxyphenyl)-N-hydroxy-3-phthalimidopropionamide;
3-(3-ethoxy-4-methoxyphenyl)-N-hydroxy-3-(1,3-dioxo-2,3-dihydro-1H-benzo[f]isoindol-2-yl)propionamide;
N-hydroxy-3-{3-(2-propoxy)-4-methoxyphenyl}-3-phthalimidopropionamide;
3-(3-ethoxy-4-methoxyphenyl)-3-(3,6-difluorophthalimido)-N-hydroxypropionamide;
3-(4-aminophthalimido)-3-(3-ethoxy-4-methoxyphenyl)-N-hydroxypropionamide;
3-(3-aminophthalimido)-3-(3-ethoxy-4-methoxyphenyl)-N-hydroxypropionamide;
N-hydroxy-3-(3,4-dimethoxyphenyl)-3-(1-oxoisoindolinyl)propionamide;
3-(3-cyclopentyloxy-4-methoxyphenyl)-N-hydroxy-3-(i-oxoisoindolinyl) propionamide: and
N-benzyloxy-3-(3-ethoxy4-methoxyphenyl)-3-(3-nitrophthalimido)propionamide.

Additional selective cytokine inhibitory drugs used in the invention include the substituted phenethylsulfones substituted on the phenyl group with a oxoisoindine group. Examples of such compounds include, but are not limited to, those disclosed in U.S. Pat. No. 6,020,358, which is incorporated herein, which include the following: 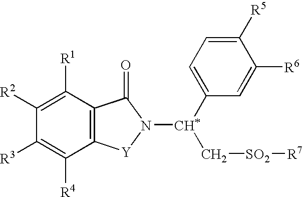
wherein the carbon atom designated * constitutes a center of chirality;
Y is C═O, CH2, SO₂, or CH₂C═O; each of R¹, R², R³, and R⁴, independently of the others, is hydrogen, halo, alkyl of 1 to 4 carbon atoms, alkoxy of 1 to 4 carbon atoms, nitro, cyano, hydroxy, or —NR⁸R⁹; or any two of R¹, R², R³, and R⁴ on adjacent carbon atoms, together with the depicted phenylene ring are naphthylidene;
each of R⁵ and R⁶, independently of the other, is hydrogen, alkyl of 1 to 4 carbon atoms, alkoxy of 1 to 4 carbon atoms, cyano, or cycloalkoxy of up to 18 carbon atoms;
R⁷ is hydroxy, alkyl of 1 to 8 carbon atoms, phenyl, benzyl, or NR^{8′}R^{9′};
each of R⁸ and R⁹ taken independently of the other is hydrogen, alkyl of 1 to 8 carbon atoms, phenyl, or benzyl, or one of R⁸ and R⁹ is hydrogen and the other is —COR¹⁰ or —SO₂R¹⁰, or R⁸ and R⁹ taken together are tetramethylene, pentamethylene, hexamethylene, or —CH₂CH₂X¹CH₂CH₂— in which X¹ is —O—, —S— or —NH—; and
each of R^{8′} and R^{9′} taken independently of the other is hydrogen, alkyl of 1 to 8 carbon atoms, phenyl, or benzyl, or one of R^{8′} and R^{9′} is hydrogen and the other is —COR¹⁰ or —SO₂R^{10′}, or R^{8′} and R^{9′} taken together are tetramethylene, pentamethylene, hexamethylene, or —CH₂CH₂X²CH₂CH₂— in which X² is —O—, —S—, or —NH—.

It will be appreciated that while for convenience the above compounds are identified as phenethylsulfones, they include sulfonamides when R⁷ is NR⁸R^{9′}.

Specific groups of such compounds are those in which Y is C═O or CH₂.

A further specific group of such compounds are those in which each of R¹, R², R³, and R⁴ independently of the others, is hydrogen, halo, methyl, ethyl, methoxy, ethoxy, nitro, cyano, hydroxy, or —NR⁸R⁹ in which each of R⁸ and R⁹ taken independently of the other is hydrogen or methyl or one of R⁸ and R⁹ is hydrogen and the other is —COCH₃.

Particular compounds are those in which one of R¹, R², R³, and R⁴ is —NH₂ and the remaining of R¹, R², R³, and R⁴ are hydrogen.

Particular compounds are those in which one of R¹, R², R³, and R⁴ is —NHCOCH₃ and the remaining of R¹, R², R³, and R⁴ are hydrogen.

Particular compounds are those in which one of R¹, R², R³, and R⁴ is —N(CH₃)₂ and the remaining of R¹, R², R³, and R⁴ are hydrogen.

A further preferred group of such compounds are those in which one of R¹, R², R³, and R⁴ is methyl and the remaining of R¹, R², R³, and R⁴ are hydrogen.

Particular compounds are those in which one of R¹, R², R³, and R⁴ is fluoro and the remaining of R¹, R², R³, and R⁴ are hydrogen.

Particular compounds are those in which each of R⁵ and R⁶, independently of the other, is hydrogen, methyl, ethyl, propyl, methoxy, ethoxy, propoxy, cyclopentoxy, or cyclohexoxy.

Particular compounds are those in which R⁵ is methoxy and R⁶ is monocycloalkoxy, polycycloalkoxy, and benzocycloalkoxy.

Particular compounds are those in which R⁵ is methoxy and R⁶ is ethoxy.

Particular compounds are those in which R⁷ is hydroxy, methyl, ethyl, phenyl, benzyl, or NR^{8′}R^{9′} in which each of R^{9′} and R^{9′} taken independently of the other is hydrogen or methyl.

Particular compounds are those in which R⁷ is methyl, ethyl, phenyl, benzyl or NR^{8′}R^{9′} in which each of R^{8′} and R^{9′} taken independently of the other is hydrogen or methyl.

Particular compounds are those in which R⁷ is methyl.

Particular compounds are those in which R⁷ is NR^{8′}R^{9′} in which each of R^{8′} and R^{9′} taken independently of the other is hydrogen or methyl.

Other specific selective cytokine inhibitory drugs include fluoroalkoxy-substituted 1,3-dihydro-isoindolyl compounds found in U.S. Provisional Application No. 60/436,975 to G. Muller et al., filed Dec. 30, 2002, which is incorporated herein in its entirety by reference. Representative fluoroalkoxy-substituted 1,3-dihydro-isoindolyl compounds include compounds of the formula: 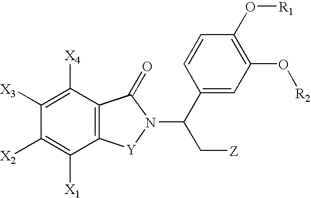
wherein:
Y is —C(O)—, —CH₂, —CH₂C(O)—, —C(O)CH₂—, or SO₂;
Z is —H, —C(O)R³, —(C₀₋₁-alkyl)-SO₂—(C₁₋₄-alkyl), —C₁₋₈-alkyl, —CH₂OH, CH₂(O)(C₁₋₈-alkyl) or —CN;
R₁ and R₂ are each independently —CHF₂, —C₁₋₈-alkyl, —C₃₋₁₈-cycloalkyl, or —(C₁₋₁₀-alkyl)(C₃₋₁₈-cycloalkyl), and at least one of R₁ and R₂ is CHF₂;
R³ is —NR⁴R⁵, -alkyl, —OH, —O-alkyl, phenyl, benzyl, substituted phenyl, or substituted benzyl;
R⁴ and R⁵ are each independently-H, —C₁₋₈-alkyl, —OH, —OC(O)R⁶;
R⁶ is C₁₋₄-alkyl, -amino(C₁₋₈-alkyl), -phenyl, -benzyl, or -aryl;
X₁, X₂, X₃, and X₄ are each independent —H, -halogen, -nitro, —NH₂, —CF₃, —C₁₋₆-alkyl, —(C₀₋₄-alkyl)-(C₃₋₆-cycloalkyl), (C₀₋₄-alkyl)-NR⁷R⁸, (C₀₋₄-alkyl)-N(H)C(O)—(R⁸), (C₀₋₄-alkyl)—N(H)C(O)N(R⁷R⁸), (C₀₋₄-alkyl)-N(H)C(O)O(R⁷R⁸), (C₀₋₄-alkyl)-OR⁸, (C₀₋₄-alkyl)-imidazolyl, (C₀₋₄-alkyl)-pyrrolyl, (C₀₋₄-alkyl)-oxadiazolyl, or (C₀₋₄-alkyl)-triazolyl, or two of X₁, X₂, X₃, and X₄ may be joined together to form a cycloalkyl or heterocycloalkyl ring, (e.g., X₁ and X₂, X₂ and X₃, X₃ and X₄, X₁ and X₃, X₂ and X₄, or X₁ and X₄ may form a 3, 4, 5, 6, or 7 membered ring which may be aromatic, thereby forming a bicyclic system with the isoindolyl ring); and
R⁷ and R⁸ are each independently H, C₁₋₉-alkyl, C₃₋₆-cycloalkyl, (C₁₋₆-alkyl)-(C₃₋₆-cycloalkyl), (C₁₋₆-alkyl)-N(R⁷R⁸), (C₁₋₆-alkyl)-OR⁸, phenyl, benzyl, or aryl;
or a pharmaceutically acceptable salt, solvate, hydrate, stereoisomer, clathrate, or prodrug thereof.

Preferred compounds include, but are not limited to:
3-(4-Acetylamino-1,3-dioxo-1,3-dihydro-isoindol-2-yl)-3-(3-cyclopropylmethoxy-4-difluoromethoxy-phenyl)-propionic acid;
3-(4-Acetylamino-1,3-dioxo-1,3-dihydro-isoindol-2-yl)-3-(3-cyclopropylmethoxy-4-difluoromethoxy-phenyl)-N,N-dimethyl-propionamide;
3-(4-Acetylamino-1,3-dioxo-1,3-dihydro-isoindol-2-yl)-3-(3 cyclopropylmethoxy-4-difluoromethoxy-phenyl)-propionamide;
3-(3-Cyclopropylmethoxy-4-difluoromethoxy-phenyl)-3-(1,3-dioxo-1,3-dihydro-isoindol-2-yl)-propionic acid;
3-(3-Cyclopropylmethoxy4-difluoromethoxy-phenyl)-3-(1,3-dioxo-1,3-dihydro-isoindol-2-yl)-N-hydroxy-propionamide;
3-(3-Cyclopropylmethoxy-4-difluoromethoxy-phenyl)-3-(7-nitro-1-oxo-1,3-dihydro-isoindol-2-yl)-propionic acid methyl ester;
3-(3-Cyclopropylmethoxy-4-difluoromethoxy-phenyl)-3-(7-nitro-1-oxo-1,3-dihydro-isoindol-2-yl)-propionic acid;
3-(3-Cyclopropylmethoxy-4-difluoromethoxy-phenyl-3-(7-nitro-1-oxo-1,3-dihydro-isoindol-2-yl)-)-N,N-dimethyl-propionamide;
3-(7-Amino-1-oxo-1,3-dihydro-isoindol-2-yl)-3-(3-cyclopropylmethoxy-4-difluoromethoxy-phenyl)-N,N-dimethyl-propionamide;
3-(4-Difluoromethoxy-3-ethoxy-phenyl)-3-(7-nitro-1-oxo-1,3-dihydro-isoindol-2-yl)-propionic acid methyl ester, 3-(7-Amino-1-oxo-1,3-dihydro-isoindol-2-yl)-3-(4-difluoromethoxy-3-ethoxy-phenyl)-propionic acid methyl ester;
3-(7-(Cyclopropanecarbonyl-amino)-1-oxo-1,3-dihydro-isoindol-2-yl]-3-(4-difluoromethoxy-3-ethoxy-phenyl)-propionic acid methyl ester;
3-(7-Acetylamino-1-oxo-1,3-dihydro-isoindol-2-yl)-3-(4-difluoromethoxy-3-ethoxy-phenyl)-propionic acid methyl ester;
3-(7-Acetylamino-1-oxo-1,3-dihydro-isoindol-2-yl)-3-(4-difluoromethoxy-3-ethoxy-phenyl)-propionic acid; 3
-[7-(Cyclopropanecarbonyl-amino)-1-oxo-1,3-dihydro-isoindol-2-yl]-3-(4-difluoromethoxy-3-ethoxy-phenyl)-propionic acid;
Cyclopropanecarboxylic acid {2-[2-carbamoyl-1-(4-difluoromethoxy-3-ethoxy-phenyl)-ethyl]-3-oxo-2,3-dihydro-1H-isoindol-4-yl}-amide;
Cyclopropanecarboxylic acid {2-[1-(4-difluoromethoxy-3-ethoxy-phenyl)-2-dimethylcarbamoyl-ethyl]-3-oxo-2,3-dihydro-1H-isoindol-4-yl}-;
Cyclopropanecarboxylic acid {2-[1-(4-difluoromethoxy-3-ethoxy-phenyl)-2-hydroxycarbamoyl-ethyl]-3-oxo-2,3-dihydro-1H-isoindol-4-yl}-amide;
3-(7-Acetylamino-1-oxo-1,3-dihydro-isoindol-2-yl)-3-(4-difluoromethoxy-3-ethoxy-phenyl)-propionamide;
3-(7-Acetylamino-1-oxo-1,3-dihydro-isoindol-2-yl)-3-(4-difluoromethoxy-3-ethoxy-phenyl)-N,N-dimethyl-propionamide;
3-(7-Acetylamino-1-oxo-1,3-dihydro-isoindol-2-yl)-3-(4-difluoromethoxy-3-ethoxy-phenyl)-N-hydroxy-propionamide;
3-(4-Acetylamino-1,3-dioxo-1,3-dihydro-isoindol-2-yl)-3-(4-difluoromethoxy-3-ethoxy-phenyl)-propionic acid;
3-(4-Acetylamino-1,3-dioxo-1,3-dihydro-isoindol-2-yl)-3-(4-difluoromethoxy-3-ethoxy-phenyl)-propionamide;
3-(4-Acetylamino-1,3-dioxo-1,3-dihydro-isoindol-2-yl)-3-(4-difluoromethoxy-3-ethoxy-phenyl)-N,N-dimethyl-propionamide;
3-(4-Acetylamino-1,3-dioxo-1,3-dihydro-isoindol-2-yl)-3-(4-difluoromethoxy-3-ethoxy-phenyl)-N-hydroxy-propionamide;
Cyclopropanecarboxylic acid {2-[1-(4-difluoromethoxy-3-ethoxy-phenyl)-2-methanesulfonyl-ethyl]-3-oxo-2,3-dihydro-1H-isoindol-4-yl}-amide;
N-{2-[1-(4-Difluoromethoxy-3-ethoxy-phenyl)-2-methanesulfonyl-ethyl]-1,3-dioxo-2,3 dihydro-1H-isoindol-4-yl}-acetamide; and
Cyclopropanecarboxylic acid {2-[2-carbamoyl-1-(4-difluoromethoxy-3-ethoxy-phenyl)-ethyl]-7-chloro-3-oxo-2,3-dihydro-1H-isoindol-4-yl}-amide.

Other selective cytokine inhibitory drugs include 7-amido-substituted isoindolyl compounds found in U.S. Provisional Application No. 60/454,155 to G. Muller et al., filed Mar. 12, 2003, which is incorporated herein in its entirety by reference. Representative 7-amido-substituted isoindolyl compounds include compounds of the formula: 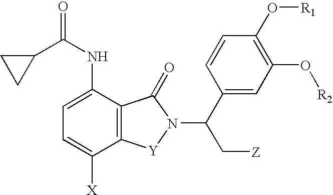
wherein:
Y is —C(O)—, —CH₂, —CH₂C(O)— or SO₂;
X is H,
Z is (C₀₋₄-alkyl)-C(O)R³, C₁₋₄-alkyl, (C₀₋₄-alkyl)-OH, (C₁₋₄-alkyl)-O(C₁₋₄-alkyl), (C₁₋₄-alkyl)-SO₂(C₁₋₄-alkyl), (C₀₋₄-alkyl)-SO(C₁₋₄-alkyl), (C₀₋₄-alkyl)-NH₂, (C₀₋₄-alkyl)—N(C₁₋₈-alkyl)₂, (C₀₋₄-alkyl)-N(H)(OH), CH₂NSO₂(C₁₋₄-alkyl);
   R₁ and R₂ are independently C₁₋₈-alkyl, cycloalkyl, or(C₁₋₄-alkyl)cycloalkyl;
   R³ is, NR⁴ R⁵, OH, or O—(C₁₋₈-alkyl);
   R⁴ is H;
   R⁵ is —OH, or —OC(O)R⁶;
   R⁶ is C₁₋₈-alkyl, amino-(C₁₋₈-alkyl), (C₁₋₈-alkyl)-(C₃₋₆-cycloalkyl), C₃₋₆cycloalkyl, phenyl, benzyl, or aryl;
   or a pharmaceutically acceptable salt, solvate, hydrate, stereoisomer, clathrate, or prodrug thereof; or the formula: 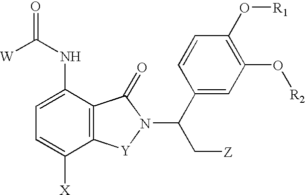
   wherein:
   Y is —C(O)—, —CH₂, —CH₂C(O)—, or SO₂;
   X is halogen, —CN, —NR₇R₈, —NO₂, or —CF₃,
   W is 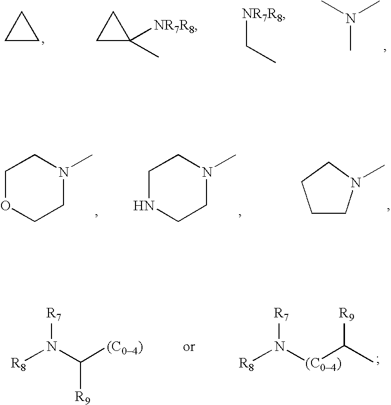
   Z is (C₀₋₄alkyl)-SO₂(C₁₋₄-alkyl), —(C₀₋₄alkyl)-CN, —(C₀₋₄alkyl)—C(O)R³, C₁₋₄-alkyl, (C₀₋₄-alkyl)OH, (C₀₋₄-alkyl)O(C₁₋₄-alkyl), (C₀₋₄-alkyl)SO(C₁₋₄-alkyl), (C₀₋₄-alkyl)NH₂, (C₀₋₄-alkyl)N(C₁₋₈-alkyl)₂, (C₀₋₄-alkyl) N(H)(OH), or (C₀₋₄-alkyl)NSO₂(C₁₋₄-alkyl);
   W is —C₃₋₆-cycloalkyl, —(C₁₋₈-alkyl)-(C₃₋₆cycloalkyl), —(C₀₋₈-alkyl)-(C₃₋₆cycloalkyl)-NR₇R₈, (C₀₋₈-alkyl)-NR₇R₈, (C₀₋₄-alkyl)-CHR₉—(C₀₋₄-alkyl)-NR₇R,
   R₁ and R₂ are independently C₁₋₈-alkyl, cycloalkyl, or (C₁₋₄-alkyl)cycloalkyl;
   R³ is C₁₋₈-alkyl, NR⁴ R⁵, OH, or O—(C₁₋₈alkyl);
   R⁴ and R⁵ are independently H, C₁₋₈-alkyl, (C₀₋₈-alkyl)-(C₃₋₆-cycloalkyl), OH, or —OC(O)R⁶
   R⁶ is C₁₋₈-alkyl, (C₀₋₈-alkyl)-(C₃₋₆-cycloalkyl), amino-(C₁₋₈-alkyl), phenyl, benzyl, or aryl;
   R₇ and R are each independently H, C₁₋₈-alkyl, (C₀₋₈alkyl)-(C₃₋₆-cycloalkyl), phenyl, benzyl, aryl, or can be taken together with the atom connecting them to form a 3 to 7 membered heterocycloalkyl or heteroaryl ring;
   R₉ is C₁₋₄-alkyl, (C₀₋₄-alkyl)aryl, (C₀₋₄-alkyl)-(C₃₋₆-cycloalkyl), (C₀₋₄-alkyl)-heterocycle;
   or a pharmaceutically acceptable salt, solvate, hydrate, stereoisomer, clathrate, or prodrug thereof

Still other selective cytokine inhibitory drugs include N-alkyl-hydroxamic acid-isoindolyl compounds found in U.S. Provisional Application No. 60/454,149 to G. Muller et al., filed Mar. 12, 2003, which is incorporated herein in its entirety by reference. Representative N-alkyl-hydroxamic acid-isoindolyl compounds include compounds of the formula: 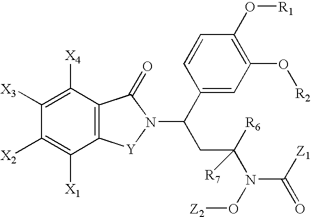
wherein:
Y is —C(O)—, —CH₂, —CH₂C(O)— or SO₂;
R₁ and R₂ are independently C₁₋₈-alkyl, CF₂H, CF₃, CH₂CHF₂, cycloalkyl, or (C₁₋₈-alkyl)cycloalkyl;
Z, is H, C₁₋₆-alkyl, —NH₂—NR₃R₄ or OR₅;
Z₂ is H or C(O)R₅;
X₁, X₂, X₃ and X₄ are each independent H, halogen, NO₂, OR₃, CF₃, C₁₋₆-alkyl, (C₀₋₄-alkyl)-(C₃₋₆-cycloalkyl), (C₀₋₄-alkyl)-N—(R₈R₉), (C₀₋₄-alkyl)-NHC(O)—(R₉), (C₀₋₄-alkyl)-NHC(O)CH(R₉)(R₉), (C₀₋₄-alkyl)-NHC(O)N(R₉), (C₀₋₄alkyl)-NHC(O)O(R₅), (C₀₋₄-alkyl)-O—R₈(C₀₋₄-alkyl)-imidazolyl, (C₀₋₄-alkyl)-pyrrolyl, (C₀₋₄-alkyl)-oxadiazolyl, (C₀₋₄-alkyl)-triazolyl or (C₀₋₄-alkyl)-heterocycle;
R₃, R₄, and R₅ are each independently H, C₁₋₆-alkyl, O-C₁₋₆-alkyl, phenyl, benzyl, or aryl;
R₆ and R₇ are independently H or C₁₋₆-alkyl;
R₈ and R₉ are each independently H, C₁₋₉-alkyl, C₃₋₄-cycloalkyl, (C₁₋₆-alkyl)-(C₃₋₆-cycloalkyl), (C₀₋₆-alkyl)—N(R₄R₅), (C₁₋₆-alkyl)-OR₅, phenyl, benzyl, aryl, piperidinyl, piperizinyl, pyrolidinyl, morpholino, or C₃₋₇-heterocycloalkyl; and
or a pharmaceutically acceptable salt, solvate, hydrate, stereoisomer, clathrate, or prodrug thereof.

Specific selective cytokine inhibitory drugs include, but are not limited to:
2-[1 (−3-ethoxy-4-methoxyphenyl)-2-methyl-sulfonylethyl]isoindolin-1-one;
2-[1-(3-ethoxy-4-methoxyphenyl)-2-(N,N-dimethyl-aminosulfonyl)ethyl]isoindolin-1-one;
2-[1-(3-ethoxy-4-methoxyphenyl)-2-methyl-sulfonylethyl]isoindoline-1,3-dione;
2-[1-(3-ethoxy-4-methoxyphenyl)-2-methyl-sulfonylethyl]-5-nitro-isoindoline-1,3-dione;
2-[1-(3-ethoxy-4-methoxyphenyl)-2-methyl-sulfonylethyl]4-nitro-isoindoline-1,3-dione;
2-[1-(3-ethoxy-4-methoxyphenyl)-2-methyl-sulfonylethyl]-4-aminoisoindoline-1,3-dione;
2-[1-(3-ethoxy-4-methoxyphenyl)-2-methylsulfonylethyl]-S-methylisoindoline-1,3-dione;
2-[1-(3-ethoxy-4-methoxyphenyl)-2-methylsulfonylethyl]-5-acetamidoisoindoline-1,3-dione;
2-[1-(3-ethoxy-4-methoxyphenyl)-2-methylsulfonylethyl]-4-dimethylaminoisondoline-1,3-dione;
2-[1-(3-ethoxy-4-methoxyphenyl)-2-methylsulfonylethyl]-5-dimethylaminoisoindoline-1,3-dione;
2-[1-(3-ethoxy-4-methoxyphenyl)-2-methylsulfonylethyl]benzo [e]isoindoline-1,3-dione;
2-[1-(3-ethoxy-4-methoxyphenyl)-2-methylsulfonylethyl]-4-methoxyisoindoline-1,3-dione;
1-(3-cyclopentyloxy-4-methoxyphenyl)-2-methylsulfonylethyl-amine;
2-[1-(3-cyclopentyloxy-4-methoxyphenyl)-2-methylsulfonylethyl]isoindoline-1,3-dione; and
2-[1-(3-cyclopentyloxy-4-methoxyphenyl)-2-methylsulfonylethyl]-4-dimethylaminoisoindoline-1,3-dione.

Additional selective cytokine inhibitory drugs include the enantiomerically pure compounds disclosed in U.S. provisional patent application Nos. 60/366,515 and 60/366,516 to G. Muller et al., both of which were filed Mar. 20, 2002, and U.S. provisional patent application Nos. 60/438,450 and 60/438,448 to G. Muller et al., both of which were filed on Jan. 7, 2003, and all of which are incorporated herein by reference. Preferred compounds include an enantiomer of 2-[1-(3-ethoxy-4-methoxyphenyl)-2-methylsulfonylethyl]-4-acetylaminoisoindoline-1,3-dione and an enantiomer of 3-(3,4-dimethoxy-phenyl)-3-(1-oxo-1,3-dihydro-isoindol-2-yl)-propionamide.

Preferred selective cytokine inhibitory drugs used in the invention are 3-(3,4-dimethoxy-phenyl)-3-(1-oxo-1,3-dihydro-isoindol-2-yl)-propionamide and cyclopropanecarboxylic acid {2-[1-(3-ethoxy-4-methoxy-phenyl)-2-methanesulfonyl-ethyl]-3-oxo-2,3-dihydro-1 H-isoindol-4-yl}-amide, which are available from Celgene Corp., Warren, N.J. 3-(3,4-dimethoxy-phenyl)-3-(1-oxo-1,3-dihydro-isoindol-2-yl)-propionamide has the following chemical structure:

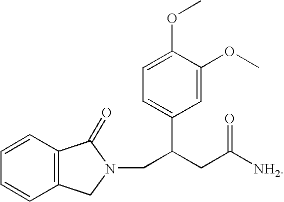

Cyclopropanecarboxylic acid {2-[1-(3-ethoxy-4-methoxy-phenyl)-2-methanesulfonyl-ethyl]-3-oxo-2,3-dihydro-1 H-isoindol-4-yl}-amide has the following chemical structure:

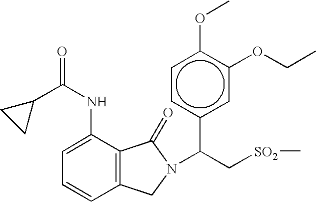

The compounds of the invention also include, but are not limited to, compounds that inhibit PDE IV activity, such as cilomast, theophylline, zardaverine, rolipram, pentoxyfylline, enoximone, isoindole-imides, phenethylsulfones, alkanohydroxamic acids, non-polypeptide cyclic amides, oxoisoindoles, isoindolines, indazoles, heterosubstituted pyridines, diphenylpyridines, aryl thiophenes, aryl furans, indenes, trisubstituted phenyls, phthalazinones, benzenesulfonamides, tetracyclic compounds and salts, solvates, isomers, clathrates, pro-drugs, hydrates or derivatives thereof. In one embodiment, the compound is not a polypeptide, peptide, protein, hormone, cytokine, oligonucleotide or nucleic acid.

In another embodiment, the compounds of this invention have the following structure (I): 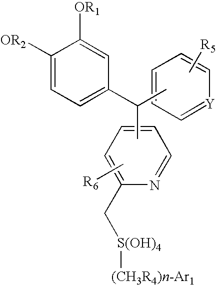
including isomers, prodrugs and pharmaceutically acceptable salts, hydrates, solvates, clathrates thereof, wherein:
Y represents N or N-oxide;
R₁ and R₂ are independently selected from:
H, C₁₋₆ alkyl and halo C₁₋₆ alkyl;
R₃ and R₄ are independently selected from H and C₁₋₆ alkyl, or R₃ and R₄ attached to the same carbon atom taken together represent a carbonyl oxygen atom, or R₃ and R₄ attached to different carbon atoms considered in combination with the carbon atoms to which they are attached along with any intervening atoms and represent a saturated 5, 6 or 7 membered carbocyclic ring;
R₅ and Rr independently represent a member selected from the group consisting of: H, C₁6 ayl, halo C₁₋₆ alkyl and CN;
n represents an integer of from 0-6;
Ar₁ is selected from the group consisting of:
thienyl, thiazolyl, pyridyl, phenyl and naphthyl; said Ar₁ being optionally substituted with 1-3 members selected from the group consisting of: halo, C₁₋₆ alkoxy, C₁₋₇ alkylthio, CN,
C₁₋₆ alkyl, hydroxy C₁₋₆ alkyl, —C(O)C₁₋₆ alkyl, —CO₂H, —CO₂C₁₋₆ alkyl, NH(SO₂Me), N(SO₂Me)₂, SO₂Me, SO₂ NH₂, SO₂NHC₁₋₆ alkyl, SO₂ N(C₁₋₆alkyl)₂ NO₂, C₂₋₆alkenyl,
C₁₋₆alkyl, and NH₂;
and when Ar₁ represents a phenyl or naphthyl group with two or three substituents, two such substituents may be considered in combination and represent a 5 or 6 membered fused lactone ring.

This embodiment further encompasses compounds such as those found in U.S. Pat. No. 6,316,472, which is incorporated herein by reference in its entirety.

In another embodiment, the compounds of the invention have the following structure (II): 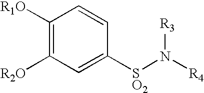
including isomers, prodrugs and pharmaceutically acceptable salts, hydrates, solvates, clathrates thereof, wherein:
R₁ and R₂ represent C₁-C₄ alkyl or C₃-C₁₀ cycloalkyl;
R₃ and R₄ independently represent C₁₋₄ alkyl, cycloalkyl, C₂-C₄ alkylenes having one double bond, C₂-C₄ alkylynes having one triple bond, (CH₂)ₙ CO(CH₂)ₘ CH₃, (CH₂)ₚ CN, (CH₂)ₚCO₂ Me, or taken together with nitrogen atom to which they are attached, form a 3-to 10-membered ring;
n and m are 0 to 3;
p is 1 to 3.

This embodiment further encompasses compounds such as those found in U.S. Pat. No. 6,162,830, which is incorporated herein by reference in its entirety.

In another embodiment, the compounds of this invention have the following structure (HI): 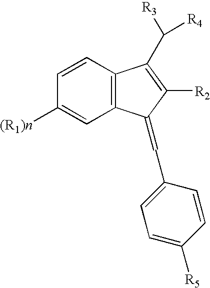
including isomers, prodrugs and pharmaceutically acceptable salts, hydrates, solvates, clathrates thereof, wherein:
R₁ is independently selected in each instance from the group consisting of hydrogen, halogen, lower alkoxy, hydroxy, lower alkyl, lower alkyl mercapto, lower alkylsulfonyl, lower alkylamino, di-lower alkyl amino, amino, nitro, nitrile, lower alkyl carboxylate, —CO₂H, and sulfonamido;
R₂ is selected from the group consisting of hydrogen and lower alkyl;
R₃ is selected from the group consisting of hydrogen, lower alkyl, hydroxy, and amino;
R₄ is selected from the group consisting of —COM and CH₂OH wherein M is selected from the group consisting of:
hydroxy, substituted lower alkoxy, amino, alkylamino, dialkylamino, N-morpholino, hydroxyalkylamino, polyhydroxyamino, dialkylaminoalkylamino, aminoalklyamino, and the group OMe, wherein Me is a cation;
R₅ is an alkyl sulfonyl; and
n is an integer from 0 to four.

This embodiment further encompasses compounds disclosed in U.S. Pat. No. 6,177,471, which is incorporated herein by reference in its entirety.

In another embodiment, the compounds of this invention have the following structure (IV): 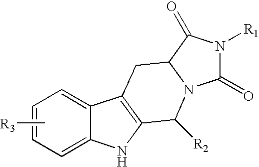
including isomers, prodrugs and pharmaceutically acceptable salts, hydrates, solvates, clathrates thereof, wherein:
R₀ represents hydrogen, halogen, or C₁₋₆ alkyl;
R₁ is selected from the group consisting of:
hydrogen; C₁₋₆ alkyl optionally substituted by one or more substituents selected from phenyl, halogen, —CO₂ Rₐ, —NRₐ R_{b}, C₃₋₆-cycloalkyl, phenyl, and a 5- or 6-membered heterocyclic ring selected from the group consisting of pyridyl, morpholinyl, piperazinyl, pyrrolidinyl, and piperidinyl, and being optionally substituted by one or more C₁₋₆ alkyl, and optionally linked to the nitrogen atom to which R₁ is attached via C₁₋₆ alkyl;
R₂ is selected from the group consisting of:
phenyl optionally substituted by one or more substituents selected from —ORₐ, —NRₐ, R_{b}, halogen, hydroxy, trifluoromethyl, cyano, and nitro;
and Rₐ and R_{b} independently represent hydrogen or C₁ 6 alkyl
including isomers, prodrugs and pharmaceutically acceptable salts thereof.

This embodiment further encompasses compounds such as those found in U.S. Pat. No. 6,218,400, which is incorporated herein by reference in its entirety.

In another embodiment, the compounds of this invention have the following structure (V): 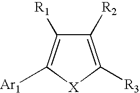
including isomers, prodrugs and pharmaceutically acceptable salts, hydrates, solvates, clathrates thereof, wherein:
X is S or O;
Ar₁ is an aromatic ring selected from phenyl, pyridinyl, or furyl, optionally substituted with up to two substituents, each substituent independently is:
C₁₋₆ alkyl, optionally substituted with —OH, —CO₂H, CO₂C₁₋₃ alkyl, or CN; C₁₋₆ alkoxy; C₁₋₃ alkylthio, C₁₋₃ alkylsulfonyl, C₁₋₃ fluoroalkyl, optionally substituted with H; halo, —OH, —CO₂H, or —CO₂C₁₋₃ alkyl;
R₂ is hydrogen or C₁₋₃ alkyl; and
R³ is phenyl, pyridinyl, quinolinyl or furyl, optionally substituted with up to two substituents, each substituent independently is: C₁₋₃ alkyl, C₁₋₃ fluoroalkyl, C₁₋₆ alkoxy, C₁₋₃ fluoroalkoxy, C₁₋₃ alkylthio, halo, or —OH.

This embodiment further encompasses compounds such as those found in U.S. Pat. No. 6,034,089 and U.S. Pat. No. 6,020,339, which are incorporated herein by reference in their entireties.

In another embodiment, the compounds of this invention have the following structure (VI): 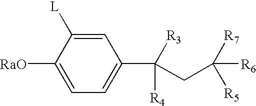
including isomers, prodrugs and pharmaceutically acceptable salts, hydrates, solvates, clathrates thereof, wherein:
Y is halogen or an alkyl or —XRₐ group;
Z is —S, (O)ₚ— or —N(R_{b})—, where p is zero or an integer 1 or 2;
L is —XR, —C(₁)C(R₁₁)(R₂) or —(CHR₁₁)ₙ CH(R₁)(R₂), where n is zero or the integer 1;
each of Rₐ and R_{b} is independently hydrogen or an optionally substituted alkyl group;
R is an optionally substituted alkyl, alkenyl, cycloalkyl or cycloalkenyl group; each of R₁ and R₂, which may be the same or different, is hydrogen, fluorine, —CN, —NO₂, or an optionally
substituted alkyl, alkenyl, alkynyl, alkoxy, alkylthio, —CO₂ R₈, —CONR₉ R₁₀ or —CSNR₉R₁₀ group, or R₁ and R₂, together with the carbon atom to which they are attached, are linked to form an optionally substituted cycloalkyl or cycloalkenyl group;
R₃ is hydrogen, fluorine, hydroxy or an optionally substituted straight or branched alkyl group;
R₄ is hydrogen, —(CH₂)ₜ Ar or —(CH₂)ₜ—Ar-(L₁)ₙ-Ar₁, where t is zero or an integer 1, 2 or 3;
R₅ is CH₂)ₜ Ar or —(CH₂)ₜ—Ar-L₁)ₙ-Ar′;
R₆ is hydrogen, fluorine, or an optionally substituted alkyl group;
R₇ is hydrogen, fluorine, an optionally substituted straight or branched alkyl group, —ORc, where Rc is hydrogen or an optionally substituted alkyl or alkenyl group, or a formyl, alkoxyalkyl, alkanoyl, carboxamido or thiocarboxamido group;
each of R₈, R₉ and R₁₀ is independently hydrogen or an optionally substituted alkyl, aralkyl or aryl group; and
R₁₁ is hydrogen, fluorine or a methyl group.

This embodiment further encompasses compounds such as those found in U.S. Pat. No. 5,798,373, which is incorporated herein by reference in its entirety.

In a preferred embodiment, the compound is of structure (VII): 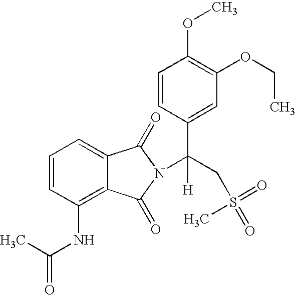
or a pharmaceutically acceptable salt, hydrate, solvate, clathrate, enantiomer, diastereomer, racemate, or mixture of stereoisomers thereof.

In another preferred embodiment, the compound is that of structure (VIII): 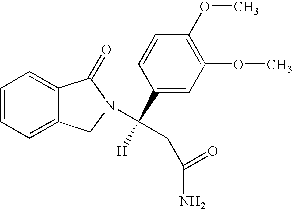
including isomers, salts, clathrates, solvates, hydrates, prodrugs and pharmaceutically acceptable salts thereof.

Certain of these compounds may be commercially available from Celgene, Inc., Warren, N.J. Other above compounds can be made by methods known in the art, including those disclosed in the patents cited above which are incorporated by reference in their entireties.

Additional examples of PDE IV inhibitors which are useful in the methods of the present invention include those disclosed in GB 2 063 249 A, EP 0 607 439 A1, U.S. Pat. No. 6,333,354, U.S. Pat. No. 6,300,335, U.S. Pat. No. 6,166,041, U.S. Pat. No. 6,069,156, U.S. Pat. No. 6,011,060, U.S. Pat. No. 5,891,896, U.S. Pat. No. 5,849,770, U.S. Pat. No. 5,710,170, U.S. Pat. No. 4,101,548, U.S. Pat. No. 4,001,238, U.S. Pat. No. 4,001,237, U.S. Pat. No. 4,101,548, U.S. Pat. No. 4,001,238, U.S. Pat. No. 4,001,237, U.S. Pat. No. 3,920,636, U.S. Pat. No. 4,060,615, WO 97/03985, EP 0 395 328, U.S. Pat. No. 4,209,623, EP 0 395 328, U.S. Pat. No. 4,209,623, U.S. Pat. No. 5,354,571, EP 0 428 268 A 2, U.S. Pat. No. 5,354,571, EP 0 428 268 A2, 807,826, U.S. Pat. No. 3,031,450, U.S. Pat. No. 3,322,755, U.S. Pat. No. 5,401,774,807,826, U.S. Pat. No. 3,031,450, U.S. Pat. No. 3,322,755, U.S. Pat. No. 5,401,774, U.S. Pat. No. 5,147,875, PCT WO 93/12095, U.S. Pat. No. 5,147,875, PCT WO 93/12095, U.S. Pat. No. 4,885,301, WO 93/07149, EP 0 349 239 A2, EP 0 352 960 A2, EP 0526 004 A1, EP 0 463 756 A1, U.S. Pat. No. 4,885,301, WO 93/07149, EP 0 349 239 A2, EP 0352 960 A2, EP 0 526 004 A1, EP 0 463 756 A1, EP 0 607 439 A1, EP 0 607 439 A1, WO 94/05661, EP 0 351 058, U.S. Pat. No. 4,162,316, EP 0 347 146, U.S. Pat. No. 4,047,404, U.S. Pat. No. 5,614,530, U.S. Pat. No. 5,488,055, WO 97/03985, WO 97/03675, WO 95/19978, U.S. Pat. No. 4,880,810, WO 98/08848, U.S. Pat. No. 5,439,895, U.S. Pat. No. 5,614,627, PCT US94/01728, WO 98/16521, EP 0 722 943 A1, EP 0 722 937 A1, EP 0 722 944 A1, WO 98/17668, WO 97/24334, WO 97/24334, WO 97/24334, WO 97/24334, WO 97/24334, WO 98/06722, PCT/JP97/03592, WO 98/23597, WO 94/29277, WO 98/14448, WO 97/03070, WO 98/38168, WO 96/32379 and PCT/GB98/03712, all of which are incorporated herein by reference.

Many of the compounds that are contemplated as part Of the present invention can be enriched in optically active enantiomers of the compounds specified above using standard resolution or asymmetric synthesis known in the art. See, e.g., Shealy et al., Chem. Indus. 1030 (1965); and Casini et al., Farmaco Ed. Sci. 19:563 (1964).

The present invention also pertains to the physiologically acceptable non-toxic acid addition salts of the compounds thereof. Such salts include those derived from organic and inorganic acids or bases know in the art: such acids include for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds of the invention that are acidic in nature are capable of forming salts with various pharmaceutically acceptable bases. The bases that can be used to prepare pharmaceutically acceptable base addition salts of such acidic compounds of the invention are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The compounds of the invention can be assayed for their ability to inhibit PDE IV using methods well known in the art, for example, those assays disclosed in U.S. Pat. No. 6,316,472; U.S. Pat. No. 6,204,275; Featherstone R. L. et al. (2000) “Comparison of phosphodiesterase inhibitors of differing isoenzyme selectivity added to St. Thomas' hospital cardioplegic solution used for hypothermic preservation of rat lungs”, Am. J. Respir Crit. Care Med. 162:850-6; and Brackeen M. F. et al. (1995) “Design and synthesis of conformationally constrained analogues of 4-(3-butoxy-4-methoxybenzyl) imidazolidin-2-one (Ro 20-1724) as potent inhibitors of cAMP-specific phosphodiesterase”, J. Med. Chem. 38:4848-54, which are incorporated herein by reference in their entirety.

The compounds of the invention can either be commercially purchased or prepared according to the methods described in the patents or patent publications disclosed herein. Further, optically pure compositions can be asymmetrically synthesized or resolved using known resolving agents or chiral columns as well as other standard synthetic organic chemistry techniques.

4.4. Methods of Stem Cell Culture

In certain embodiments of the invention, stem or progenitor cells, including but not limited to embryonic stem cells, embryonic-like stem cells, progenitor cells, pluripotent cells, totipotent cells, multipotent cells, cells endogenous to a postpartum perfused placenta, cord blood cells, stem or progenitor cells derived from peripheral blood or adult blood, or bone marrow cells, are exposed to the compounds of the invention and induced to differentiate. These cells may be propagated in vitro using methods well known in the art, or alternatively, may be propagated in a postpartum perfused placenta.

In certain embodiments, cells endogenous to a postpartum perfused placenta may be collected from the placenta and culture medium and cultured in vitro under conditions appropriate, and for a time sufficient, to induce differentiation to the desired cell type or lineage. See U.S. Application Publication No. U.S. 20030032179, published Feb. 13, 2003, entitled “Post-Partum Mammalian Placenta, Its Use and Placental Stem Cells Therefrom” which is hereby incorporated in its entirety.

In another embodiment of the invention, the stem or progenitor cells are not derived from a postpartum perfused placenta but instead, are isolated from other sources such as cord blood, bone marrow, peripheral blood or adult blood, are exposed to the compounds of the invention and induced to differentiate. In a preferred embodiment, the differentiation is conducted in vitro under conditions appropriate, and for a time sufficient, to induce differentiation into the desired lineage or cell type. The compounds of the invention are used in the differentiation/culture media by addition, in situ generation, or in any other manner that permits contact of the stem or progenitor cells with the compounds of the invention.

In another embodiment, the cultured stem cells, e.g., stem cells cultured in vitro or in a postpartum perfused placenta, are stimulated to proliferate in culture, for example, by administration of erythropoietin, cytokines, lymphokines, interferons, colony stimulating factors (CSFs), interferons, chemokines, interleukins, recombinant human hematopoietic growth factors including ligands, stem cell factors, thrombopoietin (Tpo), interleukins, and granulocyte colony-stimulating factor (G-CSF) or other growth factors.

After collection and/or isolation of the cultured cells, they may be identified and characterized by a colony forming unit assay, which is commonly known in the art, such as Mesen Cult™ medium (stem cell Technologies, Inc., Vancouver British Columbia).

Methods for culturing stem or progenitor cells in vitro are well known in the art, e.g., see, Thomson et al., 1998, Science 282:1145-47 (embryonic stem cells); Hirashima et al., 1999, Blood 93(4): 1253-63, and. Hatzopoulos et al., 1998, Development 125:1457-1468 (endothelial cell progenitors); Slager et al., 1993, Dev. Genet. 14(3):212-24 (neuron or muscle progenitors); Genbachev et al., 1995, Reprod. Toxicol. 9(3):245-55 (cytotrophoblasts, i.e., placental epithelial cell progenitors); Nadkarni et al. 1984, Tumori 70:503-505, Melchner et al., 1985, Blood 66(6): 1469-1472, international PCT publication WO 00/27999 published May 18, 2000, Himori et al., 1984, Intl. J. Cell Cloning 2:254-262, and Douay et al., 1995, Bone Marrow Transplantation 15:769-775 (hematopoietic progenitor cells); Shamblott et al., 1998, Proc. Natl. Acad. Sci. USA 95:13726-31 (primordial germ cells); Yan et al., 2001, Devel. Biol. 235:422-432 (trophoblast stem cells). Such methods may be easily adapted for use in the methods of the invention, provided that the culture of the progenitor cells includes a step or steps of culturing the cells with a compound of the invention, at the times indicated, to produce the desired population(s) of differentiated cells.

4.4.1. Stem Cell Culture In Vitro

The methods of the invention encompass the regulation of stem cell or progenitor cell differentiation in vitro, comprising incubating the cells with a compound, such as a small organic molecule of the present invention, in vitro, that induces them to differentiate into cells of a particular desired cell lineage, followed by direct transplantation of the differentiated cells to a subject. In a preferred embodiment, the cells are induced to differentiate into a hematopoietic cell lineage.

In certain embodiments, the cultured stem cells of interest are exposed in vitro to a 0.1 μg/ml, 0.2 μg/ml, 0.3 μg/ml, 0.4 μg/ml, 0.5 μg/ml, 1 μg/ml, 5 tug or 10 μg/ml concentration of a compound of the invention. Preferably the cells of interest are exposed to a concentration of PDE IV inhibitor of about 0.005 μg/ml to about 5 mg/ml, or a concentration of SelCID™ of about 0.005 μg/ml to about 5 mg/ml (Celgene Corp., Warren, N.J.) (see also Section 4.7, “Pharmaceutical Compositions”).

In certain embodiments, the embryonic-like stem cells are induced to propagate in the placenta bioreactor by introduction of nutrients, hormones, vitamins, growth factors, or any combination thereof, into the perfusion solution. Serum and other growth factors may be added to the propagation perfusion solution or medium. Growth factors are usually proteins and include, but are not limited to: cytokines, lymphokines, interferons, colony stimulating factors (CSFs), interferons, chemokines, and interleukins. Other growth factors that may be used include recombinant human hematopoietic growth factors including ligands, stem cell factors, thrombopoeitin (Tpo), granulocyte colony-stimulating factor (G-CSF), leukemia inhibitory factor, basic fibroblast growth factor, placenta derived growth factor and epidermal growth factor.

The growth factors introduced into the perfusion solution can stimulate the propagation of undifferentiated embryonic-like stem cells, committed progenitor cells, or differentiated cells (e.g., differentiated hematopoietic cells). The growth factors can stimulate the production of biological materials and bioactive molecules including, but not limited to, immunoglobulins, hormones, enzymes or growth factors as previously described. The cultured placenta should be “fed” periodically to remove the spent media, depopulate released cells, and add fresh media. The cultured placenta should be stored under sterile conditions to reduce the possibility of contamination, and maintained under intermittent and periodic pressurization to create conditions that maintain an; adequate supply of nutrients to the cells of the placenta. It should be recognized that the perfusing and culturing of the placenta can be both automated and computerized for efficiency and increased capacity.

4.4.2. Progenitor Cell Culture In Vitro

The methods of the invention also encompass the regulation and modulation of the development of progenitor cells, particularly CD34⁺ and CD133⁺ progenitor cells. In one embodiment of the invention, progenitor cells are induced to differentiate into a hematopoietic cell lineage. In a specific embodiment, the lineage is a granulocytic lineage. In an alternate embodiment, CD133⁺ cells are induced to differentiate into endothelial cells, brain cells, kidney cells, liver cells or intestinal tract cells.

Progenitor cells may be cultured by standard methods, as noted above. Additionally, the culture of the progenitor cells may comprise contacting the cells at various times or time frames during culture, so as to drive progenitor cell differentiation down different cell lineages.

Thus, in one method of culturing CD34⁺ or CD133⁺ progenitor cells, cells are plated at day 0 in medium containing stem cell factor (SCF), Flt-3L, GM-CSF and TNF-α and cultured for six days. On the sixth day, the cells are re-plated in medium containing GM-CSF and TNF-α, and culture is continued for an additional six days. This method results in the generation of dendritic cells. In a variation of this method, the cells are initially plated in medium containing GM-CSF and IL-4, then switched on the sixth day to monocyte-conditioned medium (see Steinman et al., International Publication No. WO 97/29182). To produce a population of CD34+CD38⁻CD33⁺ or CD34⁺CD38⁻CD33⁻ progenitor cells, the progenitor cells are placed in contact with a compound of the invention at day 0, and CD34⁺CD38⁻CD33⁺ or CD34⁺CD38⁻CD33⁻ progenitor cells are collected at day 6.

The timing of the addition of the compound(s) of the invention, particularly SelCIDs™, is expected to have a substantial effect upon the path of differentiation of CD34⁺ cells into cells of particular lineages, and on the differentiation of CD 133+cells. CD34⁺ progenitor cells, cultured under standard conditions; follow a myeloid developmental pathway or lineage, i.e., become dendritic cells within 12 days after initial plating (i.e., after initial culture). However, the addition of a compound of the invention at one of several particular times during the first six days of culture substantially alters this pathway. For example, if CD34⁺ cells, particularly CD34⁺ derived from bone marrow, are exposed to a compound of the invention, particularly SelCIDs™ on the first day of culture, differentiation along the myeloid lineage would be suppressed, as evidenced by the increase in the number of CD34⁺CD38⁻ cells and decrease in the number of CD1a⁺CD 14− cells at day 6 of culture, relative to a control not exposed to a compound of the invention (i.e., exposed to DMSO). Moreover, exposure to a compound of the invention would lead to suppression of the development of cells expressing surface markers expressed by cells in a dendritic cell lineage, such as CD80 and CD86. Contact at the initial day of culture, or at any point up to three days after the initial day of culture, with a compound of the invention, would leads to such modulation of the development of CD34⁺ progenitor cells. The increase in the number of CD34⁺ cells will be intensified if multiple doses of a compound of the invention are given between day 0 and day 6, for example, doses at day 0 and day 2, day 0 and day 4, doses at day 3 and day 6, or doses at day 2, day 4, and day 6.

In a particularly useful aspect of the invention, the addition of a compound of the invention at the first day of CD34⁺ progenitor cell culture, and continuing the exposure through day 12, leads to the development of a unique progenitor cell expressing a unique combination of cell surface markers: CD34⁺CD38⁻CD33⁺ or CD34⁺CD38⁻CD33⁻. The CD34⁺CD38⁻CD33⁺ or CD34⁺CD38⁻CD33⁻ cell population represents an intermediate stage in differentiation. This population is useful as an expandable population of progenitor cells that may readily be transplanted to a patient in need of a rapidly-developing population of hematopoietic lineage cells, for example, granulocytic cells. In another embodiment, CD34⁺ cells may be plated and cultured during the proliferative phase (approximately 6 days) in standard medium (i.e., not exposed to a PDE IV inhibitor, such as a SelCID™ or the like), then switched to the same or a similar medium containing a SelCID™ or prodrug thereof, or the like, and continuing the culture until day 12. In this embodiment, the differentiating cells typically show decreased expression of CD80, CD86 and CD14, but result in an increased persistence of a CD1a⁺ cell population relative to controls. Such differentiating cells are not blocked from becoming dendritic cells. In another embodiment, CD34⁺ cells are treated during the proliferative phase (days 1-6 post-plating) for at least three consecutive days with a SelCID™, or another compound of the invention. In yet another embodiment, CD34⁺ or CD133⁺ progenitor cells are treated two or more Limes with a SelCID™, or another compound of the invention, during the first six days after plating. Such multiple treatments will result in an increase in the proliferation of both CD34⁺ or CD133⁺ populations. Multiple treatments with a SelCID™, or another compound of the invention, will cause a shift in the differentiation of CD34⁺ progenitor cells away from a CD11c⁺CD15⁻ lineage and towards a CD11c⁻CD15⁺lineage, i.e., away from a myeloid dendritic cell lineage and towards a granulocytic lineage (FIG. 6B).

Treatment of the progenitor cells from day 0 of culture, particularly multiple doses between day 0 and day 6, also results in an increase in the number of CD133⁺ progenitor cells, particularly an increase in the CD34⁺CD133⁺ progenitor population. CD133 is a hematopoietic marker that is an alternative to CD34 isolation, as CD133+ cells can be expanded in the same manner as the CD34⁺ subset and conserve their multilineage capacity (see Kobari et al., J. Hematother. Stem Cell Res. 10(2):273-81 (2001)). CD133+ has been reported to be present in CD34⁻ cells from human fetal brain tissue, and showed potent engraftment, proliferation, migration, and neural differentiation when injected into neonatal mice (see Proc. Natl. Acad. Sci. U.S.A. 19:97(26):14720-5 (2000)). CD133⁺ hematopoietic stem cells have been shown to be enriched for progenitor activity with enlarged clonogenic capacity and higher engraftment in NOD-SCID mice.

The above notwithstanding, if a compound of the invention is placed in contact with proliferating CD34⁺ progenitor cells after three days of culture (i.e., at any time between 3-6 days after initial culture), the proliferating progenitor cells, which have already begun expressing the cell surface marker CD1a, show a substantially increased persistence of the expression of this marker relative to DMSO-treated controls. It is important to note that no cytotoxicity is associated with this increased persistence. In other words, treatment with a PDE IV inhibitor, such as a SelCID™^{,} will not cause other cell populations to apoptose. The net effect is a maintenance of existing immune capability and the development of new immune capability.

Thus, in one embodiment of the method of the invention, differentiation of CD34⁺ cells into dendritic cells is modulated (i.e., suppressed) by contacting CD34⁺ progenitor cells with a compound of the invention at day 0 of culture (i.e., the first day of culture). In another embodiment, differentiation of CD34⁺ cells into granulocytic cells is enhanced by contacting CD34⁺ progenitor cells with a compound of the invention at day 0 of culture (i.e., the first day of culture). In another embodiment, differentiation of CD34⁺ cells into a CD34⁺CD38⁻CD33⁺or a CD34⁺CD38⁻CD33⁻ progenitor cell population is enhanced by contacting CD34⁺ progenitor cells with a compound of the invention during the first three days of culture. In another embodiment, a CD34⁺CD133⁺ population is enhanced or increased by contacting progenitor cells with a compound of the invention in multiple doses from day 0 to day 6 of culture. In another embodiment, the persistence of a CD1a⁺ cell population is enhanced or increased by contacting CD34⁺ progenitor cells with a compound of the invention at day 6 of culture, wherein said CD34⁺ cells differentiate into cells exhibiting the CD1a surface marker, and wherein said culture includes no contact with said compound for up to six days.

In the above embodiments, it will be understood that such variations in administration of SelCIDs™, or related compounds, may be made to the progenitor cells in vivo, e.g., such as in a patient into whom such cells have been transplanted or engrafted, as well as to the progenitor cells in vitro.

The methods of the invention encompass the regulation of stem cell or progenitor cell differentiation in vitro, comprising incubating the cells with a compound, such as a small organic molecule of the present invention, in vitro, that induces them to differentiate into cells of a particular desired cell lineage, followed by direct transplantation of the differentiated cells to a subject. In a preferred embodiment, the cells are induced to differentiate into a hematopoietic cell lineage. In an alternate embodiment, CD133⁺ cells are induced to differentiate into endothelial cells, brain cells, kidney cells, liver cells, or intestinal tract cells.

It should be noted that the methods described herein are contemplated for use with CD34⁺ or CD133⁺ progenitor cells derived from mammals, preferably humans, but are also contemplated for use with avian or reptilian progenitor cells. The compounds of the invention, however, are potentially variably potent depending upon the species from which the progenitor cells are derived. Some variation in the culturing methods, particularly with regard to the concentration of the compound(s) administered, is therefore also contemplated. For example, progenitor cells of murine origin are less sensitive to the compounds of the invention, for example a SelCID™, and would require higher concentrations to achieve the effects obtainable at 1 μM with progenitor cells of human origin. Persons of skill in the art would understand that such optimizations are routine.

4.5. Genetic Engineering of Stem and Progenitor Cells

In another embodiment of the invention, stem or progenitor cells to be differentiated in accordance with the methods of the invention are genetically engineered either prior to, or after exposure to the compounds of the invention, using, for example, a viral vector such as an adenoviral or retroviral vector, or by using mechanical means such as liposomal or chemical mediated uptake of the DNA. In specific embodiments, the CD34⁺ progenitor cells are genetically engineered, then treated with a compound of the invention; in more specific embodiments, said compound is a SelCID™, or an analog thereof. In another embodiment, said cells are treated with a compound of the invention, then genetically engineered.

A vector containing a transgene can be introduced into a cell of interest by methods well known in the art, e.g., transfection, transformation, transduction, electroporation, infection, microinjection, cell fusion, DEAE dextran, calcium phosphate precipitation, liposomes, LIPOFECTIN™, lysosome fusion, synthetic cationic lipids, use of a gene gun or a DNA vector transporter, such that the transgene is transmitted to daughter cells, e.g., the daughter embryonic-like stem cells or progenitor cells produced by the division of an embryonic-like stem cell. For various techniques for transformation or transfection of mammalian cells, see Keown et al., 1990, Methods Enzymol. 185: 527-37, Sambrook et al., 2001, Molecular Cloning, A Laboratory Manual, Third Edition, Cold Spring Harbor Laboratory Press, N.Y.

Preferably, the transgene is introduced using any technique, so long as it is not destructive to the cell's nuclear membrane or other existing cellular or genetic structures. In certain embodiments, the transgene is inserted into the nucleic genetic material by microinjection. Microinjection of cells and cellular structures is commonly known and practiced in the art.

For stable transfection of cultured mammalian cells, such as cells culture in a placenta, only a small fraction of cells may integrate the foreign DNA into their genome. The efficiency of integration depends upon the vector and transfection technique used. In order to identify and select integrants, a gene that encodes a selectable marker (e.g., for resistance to antibiotics) is generally introduced into the host embryonic-like stern cell along with the gene sequence of interest. Preferred selectable markers include those that confer resistance to drugs, such as G418, hygromycin and methotrexate. Cells stably transfected with the introduced nucleic acid can be identified by drug selection (e.g., cells that have incorporated the selectable marker gene will survive, while the other cells die). Such methods are particularly useful in methods involving homologous recombination in mammalian cells (e.g., in embryonic-like stem cells) prior to introduction or transplantation of the recombinant cells into a subject or patient.

A number of selection systems may be used to select transformed host stem cells, such as embryonic-like cells, or progenitor cells, such as CD34⁺ or CD133⁺ progenitor cells. In particular, the vector may contain certain detectable or selectable markers. Other methods of selection include but are not limited to selecting for another marker such as: the herpes simplex virus thymidine kinase (Wigler et al., 1977, Cell 11: 223), hypoxanthine-guanine phosphoribosyltransferase (Szybalska and Szybalski, 1962, Proc. Natl. Acad. Sci. USA 48: 2026), and adenine phosphoribosyltransferase (Lowy et al., 1980, Cell 22: 817) genes can be employed in tk-, hgprt- or aprt- cells, respectively. Also, antimetabolite resistance can be used as the basis of selection for the following genes: dhfr, which confers resistance to methotrexate (Wigler et al., 1980, Proc. Natl. Acad. Sci. USA 77: 3567; O'Hare et al., 1981, Proc. Natl. Acad. Sci. USA 78: 1527); gpt, which confers resistance to mycophenylic acid (Mulligan and Berg, 1981, Proc. Natl. Acad. Sci. USA 78: 2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30:147).

The transgene may integrate into the genome of the cell of interest, preferably by random integration. In other embodiments the transgene may integrate by a directed method, e.g., by directed homologous recombination (ie., “knock-in” or “knock-out” of a gene of interest in the genome of cell of interest), Chappel, U.S. Pat. No. 5,272,071; and PCT publication No. WO 91/06667, published May 16, 1991; U.S. Pat. No. 5,464,764; Capecchi et al., issued Nov. 7, 1995; U.S. Pat. No. 5,627,059, Capecchi et al. issued, May 6, 1997; U.S. Pat. No. 5,487,992, Capecchi et al., issued Jan. 30, 1996).

Methods for generating cells having targeted gene modifications through homologous recombination are known in the art. The construct will comprise at least a portion of a gene of interest with a desired genetic modification, and will include regions of homology to the target locus, i.e., the endogenous copy of the targeted gene in the host's genome. DNA constructs for random integration, in contrast to those used for homologous recombination, need not include regions of homology to mediate recombination. Markers can be included in the targeting construct or random construct for performing positive and negative selection for insertion of the transgene.

To create a homologous recombinant cell, e.g., a homologous recombinant embryonic like stem cell, endogenous placental cell or exogenous cell cultured in the placenta, a homologous recombination vector is prepared in which a gene of interest is flanked at its 5′ and 3′ ends by gene sequences that are endogenous to the genome of the targeted cell, to allow for homologous recombination to occur between the gene of interest carried by the vector and the endogenous gene in the genome of the targeted cell. The additional flanking nucleic acid sequences are of sufficient length for successful homologous recombination with the endogenous gene in the genome of the targeted cell. Typically, several kilobases of flanking DNA (both at the 5′ and 3′ ends) are included in the vector. Methods for constructing homologous recombination vectors and homologous recombinant animals from recombinant stem cells are commonly known in the art (see, e.g., Thomas and Capecchi, 1987, Cell 51: 503; Bradley, 1991, Curr. Opin. Bio/Technol. 2: 823-29; and PCT Publication Nos. WO 90/11354, WO 91/01140, and WO 93/04169.

In a specific embodiment, the methods of Bonadio et al. (U.S. Pat. No. 5,942,496, entitled Methods and compositions for multiple gene transfer into bone cells, issued Aug. 24, 1999; and PCT WO95/22611, entitled Methods and compositions for stimulating bone cells, published Aug. 24, 1995) are used to introduce nucleic acids into a cell of interest, such as a stem cell, progenitor cell or exogenous cell cultured in the placenta, e.g., bone progenitor cells.

4.6. Uses of Stem Cells and Progenitor Cells Conditioned for Differentiation

4.6.1. General Uses

The stem cells and CD34⁺ and CD133⁺ progenitor of the invention may be induced to differentiate for use in transplantation and ex vivo treatment protocols. In one embodiment, the stem cell populations are differentiated to a particular cell type and genetically engineered to provide a therapeutic gene product. In another embodiment, the progenitor cell populations are expanded into early progenitor cells and genetically engineered to provide a therapeutic gene product. In another embodiment, the progenitor cell populations are differentiated to a particular cell type, such as a granulocyte, and genetically engineered to provide a therapeutic gene product.

The compounds of the invention also have utility in clinical settings in which transplantation has the principle objective of restoring bone marrow white blood cell production, such as the reversal of neutropenia and leukopenia, which result from disease and/or clinical myeloablation. The compounds also have utility in the restoration of production of early progenitor cells or granulocytes, which result from disease, various known therapeutic side effects, or myeloablation. The compounds of the invention also have utility in cases in which the suppression of red blood cell generation is preferred, without bone marrow suppression.

In certain embodiments, stem cells that have been treated with the compounds of the invention are administered along with untreated cells, such as stem cells from cord blood or peripheral blood, to a patient in need thereof. In other embodiments, CD34⁺ or CD 133+cells that have been treated with the compounds of the invention are administered along with untreated cells, such as stem cells from cord blood or peripheral blood, to a patient in need thereof. In one embodiment, CD34⁺ progenitor cells, treated from the first day of culture with a compound of the invention, are administered with untreated cells to a patient in need thereof In a more specific embodiment, the progenitor cell transferred is a CD34⁺CD38⁻CD33⁺ or a CD34⁺CD38⁻CD33⁻ progenitor cell.

Stem cells, e.g., embryonic-like or hematopoietic stem cells, or progenitor cells, the differentiation of which has been modulated according to the methods of the invention, may be formulated as an injectable (see PCT WO 96/39101, incorporated herein by reference in its entirety). In an alternative embodiment, cells and tissues, the differentiation of which has been modulated according to the methods of the invention, may be formulated using polymerizable or cross linking hydrogels as described in U.S. Pat. No. 5,709,854; 5,516,532; or 5,654,381, each of which is incorporated by reference in its entirety.

Embryonic-like stem cells may be used instead of specific classes of progenitor cells (e.g., chondrocytes, hepatocytes, hematopoietic cells, pancreatic parenchymal cells, neuroblasts, muscle progenitor cells, etc.) in therapeutic or research protocols in which progenitor cells would typically be used.

4.6.2. Tissue Replacement or Augmentation

The stem cells, particularly embryonic-like stem cells, and progenitor cells, of the invention, the differentiation of which has been modulated according to the methods of the invention, can be used for a wide variety of therapeutic protocols directed to the transplantation or infusion of a desired cell population, such as a stem cell or progenitor cell population. The stem or progenitor cells can be used to replace or augment existing tissues, to introduce new or altered tissues, or to join together biological tissues or structures.

In a preferred embodiment of the invention, stem cells, such as embryonic-like stem cells from the placenta, or progenitor cells such as hematopoietic progenitor cells, the differentiation of which has been modulated according to the methods of the invention, may be used as autologous and allogenic, including matched and mismatched HLA type, hematopoietic transplants. In accordance with the use of embryonic-like stem cells as allogenic hematopoietic transplants, it may be preferable to treat the host to reduce immunological rejection of the donor cells, such as those described in U.S. Pat. No. 5,800,539, issued Sep. 1, 1998; and U.S. Pat. No. 5,806,529, issued Sep. 15, 1998, both of which are incorporated herein by reference.

For example, embryonic-like stem cells, the differentiation of which has been modulated according to the methods of the invention can be used in therapeutic transplantation protocols, e.g., to augment or replace stem or progenitor cells of the liver, pancreas, kidney, lung, nervous system, muscular system, bone, bone marrow, thymus, spleen, mucosal tissue, gonads, or hair. Likewise, hematopoietic progenitor cells, the differentiation of which has been modulated according to the methods of the invention, may be used instead of bone marrow or endothelial progenitor cells.

Stem cells, for example embryonic-like stem cells, the differentiation of which has been modulated according to the methods of the invention, can be used for augmentation, repair or replacement of cartilage, tendon, or ligaments. For example, in certain embodiments, prostheses (e.g., hip prostheses) are coated with replacement cartilage tissue constructs grown from embryonic-like stem cells of the invention. In other embodiments, joints (e.g., knee) are reconstructed with cartilage tissue constructs grown from embryonic-like stem cells. Cartilage tissue constructs can also be employed in major reconstructive surgery for different types of joints (for protocols, see e.g., Resnick, D., and Niwayama, G., eels., 1988, Diagnosis of Bone and Joint Disorders, 2d ed., W. B. Saunders Co.).

The stem cells and progenitor cells treated according to the methods of the invention can be used to repair damage of tissues and organs resulting from disease. In such an embodiment, a patient can be administered embryonic-like stem cells to regenerate or restore tissues or organs which have been damaged as a consequence of disease, e.g., enhance immune system following chemotherapy or radiation, repair heart tissue following myocardial infarction. Stem and/or progenitor cells treated according to the methods, and with the PDE IV inhibitors, of the invention, or administered in conjunction with the PDE IV inhibitors of the invention, may be transplanted into an individual in need thereof to repair and/or replace hepatic, pancreatic or cardiac tissue.

The stem cells and progenitor cells treated according to the methods of the invention can also be used to augment or replace bone marrow cells in bone marrow transplantation. Human autologous and allogenic bone marrow transplantation is currently used as a therapy for diseases such as leukemia, lymphoma and other life-threatening disorders. The drawback of these procedures, however, is that a large amount of donor bone marrow must be removed to insure that there is enough cells for engraftment.

The embryonic-like stem cells collected according to the methods of the invention can provide stem cells and progenitor cells that would reduce the need for large bone marrow donation. It would also be, according to the methods of the invention, to obtain a small marrow donation and then expand the number of stem cells and progenitor cells culturing and expanding in the placenta before infusion or transplantation into a recipient.

The large numbers of embryonic-like stem cells and/or progenitor obtained using the methods of the invention would, in certain embodiments, reduce the need for large bone marrow donations. Approximately 1×10⁸ to 2×10⁸ bone marrow mononuclear cells per kilogram of patient weight must be infused for engraftment in a bone marrow transplantation (i.e., about 70 ml of marrow for a 70 kg donor). To obtain 70 ml requires an intensive donation and significant loss of blood in the donation process. In a specific embodiment, cells from a small bone marrow donation (e.g., 7-10 ml) could be expanded by propagation, for example in a placental bioreactor, before infusion into a recipient. The stem cells, and progenitor cells, particularly CD34⁺ or CD133⁺ progenitor cells, the differentiation of which has been modulated according to the methods of the invention, can thus provide stem and/or progenitor cells that would reduce or eliminate the need for a large bond marrow donation.

The embryonic-like stem cells isolated from the placenta may be used, in specific embodiments, in autologous or heterologous enzyme replacement therapy to treat specific diseases or conditions, including, but not limited to lysosomal storage diseases, such as Tay-Sachs, Niemarm-Pick, Fabry's, Gaucher's, Hunter's, Hurler's syndromes, as well as other gangliosidoses, mucopolysaccharidoses, and glycogenoses.

In other embodiments, the cells may be used as autologous or heterologous transgene carriers in gene therapy to correct inborn errors of metabolism such as adrenoleukodystrophy, cystic fibrosis, glycogen storage disease, hypothyroidism, sickle cell anemia, Pearson syndrome, Pompe's disease, phenylketonuria PKU), and Tay-Sachs disease, porphyrias, maple syrup urine disease, homocystinuria, mucopolysaccharidenosis, chronic granulomatous disease, and tyrosinemia or to treat cancer, tumors or other pathological conditions.

In other embodiments, the cells may be used in autologous or heterologous tissue regeneration or replacement therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, scalp (hair) transplantation, or for reconstruction of other damaged or diseased organs or tissues.

Furthermore, a small number of stem cells and progenitor cells normally circulate in the blood stream. In another embodiment, such exogenous stem cells or exogenous progenitor cells are collected by apheresis, a procedure in which blood is withdrawn, one or more components are selectively removed, and the remainder of the blood is reinfused into the donor. The exogenous cells recovered by apheresis are expanded by the methods of the invention, thus eliminating the need for bone marrow donation entirely.

In another embodiment, expansion of hematopoietic progenitor cells in accordance with the methods of the invention is used as a supplemental treatment in addition to chemotherapy. Most chemotherapy agents used to target and destroy cancer cells act by killing all proliferating cells, i.e., cells going through cell division. Since bone marrow is one of the most actively proliferating tissues in the body, hematopoietic stem cells are frequently damaged or destroyed by chemotherapy agents and in consequence, blood cell production is diminishes or ceases. Chemotherapy must be terminated at intervals to allow the patient's hematopoietic system to replenish the blood cell supply before resuming chemotherapy. It may take a month or more for the formerly quiescent stem cells to proliferate and increase the white blood cell count to acceptable levels so that chemotherapy may resume (when again, the bone marrow stem cells are destroyed).

While the blood cells regenerate between chemotherapy treatments, however, the cancer has time to grow and possibly become more resistant to the chemotherapy drugs due to natural selection. Therefore, the longer chemotherapy is given and the shorter the duration between treatments, the greater the odds of successfully killing the cancer. To shorten the time between chemotherapy treatments, embryonic-like stem cells or progenitor cells differentiated in accordance with the methods of the invention could be introduced into the patient. Such treatment would reduce the time the patient would exhibit a low blood cell count, and would therefore permit earlier resumption of the chemotherapy treatment.

In another embodiment, the human placental stem cells can be used to treat or prevent genetic diseases such as chronic granulomatous disease.

4.6.3. Amelioration of Inflammation

The stem and progenitor cells, the differentiation of which has been modulated according to the methods of the invention, may be used as general anti-inflammatory agents. The inventors have discovered that stem and progenitor cells from, for example, cord blood, when transplanted into a patient, reduce or substantially eliminate the inflammatory response. Thus, in one embodiment, the methods of the invention comprise administering to a patient having an inflammatory response, or who is likely to develop an inflammatory response, stem cells or progenitor cells whose differentiation has been modulated by one or more of the compounds of the invention. In specific embodiments, the stem cells are embryonic-like stem cells, and the progenitor cells are hematopoietic stem cells, particularly CD34⁺ or CD133⁺ progenitor cells.

The inventors have also discovered that treatment of an individual with the compounds of the inventions, i.e., SelCIDs, stimulates the development and differentiation of cells that modulate, ameliorate or reduce the inflammatory response. Thus, another embodiment of the invention comprises a method of treating an individual having an inflammatory response, or who is likely to develop an inflammatory response, comprising administering an effective dose of one or more of the Compounds of the invention to said individual. In another embodiment, the method comprises contacting stem or progenitor cells with the compounds of the invention prior to administration to said individual, then administering a therapeutically effective dose of said cells to said individual. In yet another embodiment, cell so treated may be co-administered with one or more of the compounds of the invention to said individual in therapeutically-effective doses.

In other embodiments, inflammation may be reduced by administration of other compounds in combination with the compounds and/or cells of the invention. For example, such additional compounds may comprise steroids, such as prednisone, or any of the non-steroidal anti-inflammatory agents, such as the cox-1/cox-2 inhibitors acetylsalicylic acid (aspirin), ibuprofen, acetaminophen, cox-1-specific inhibitors, or derivatives of any of these compounds. Such additional anti-inflammatory agents may be delivered by any standard route, such as intravenously, topically, intradermally, or by inhalation, and may be delivered contemporaneously with the compounds and/or cell of the invention, or at different times.

The above methods may be used to treat any disease or condition associated with, caused by, or resulting in inflammation. For example, the methods may be used to treat inflammation caused by trauma such as accidental injury. The methods may also be used to treat inflammation caused by or injury that is associated with surgical procedures, in particular vessel-related surgical procedures such as grafts of natural tissue, synthetic vascular grafts, heart valves or angioplasties. The methods may also be used to prevent stenosis or restenosis. The methods above may also be used to treat inflammation resulting from any disease or condition, including but not limited to diseases or conditions such as heart disease, atherosclerosis, allergy or hypersensitivity, immune disorder, autoimmune disorder such as arthritis, or inflammations due to infections. In addition to treating a inflammatory condition that already exists, the cells and/or compounds of the invention may be administered to an individual prophylactically, so as to reduce the occurrence of inflammation. This is particularly useful as a form of pre-operative therapy, whereby reduction of the post-operative inflammatory response improves an individual's chances for a successful outcome and reduces hospital stay time and periods of disability.

Monitoring of the effectiveness of the anti-inflammatory effect of the above treatments may be accomplished by any known methods, such as visual inspection, MRI or CAT scans, determination of systemic or local temperature, etc. Because a protein known as C-reactive protein is a marker for inflammation, the effectiveness of the above treatment methods may be monitored by assaying for a reduction in the amount of C-reactive protein in an individual, particularly in the area formerly experiencing inflammation.

4.6.4. Production of Dendritic Cell and Granulocyte Cell Populations

The compounds of the invention may be administered specifically to modulate the differentiation of stem and/or progenitor cells along a granulocytic developmental pathway versus a dendritic cell developmental pathway. In a like manner, the cell of the invention may be modulated in vivo or ex vivo to produce expanded populations of dendritic cells or granulocytes.

Dendritic cells can be used as reagents for immune-based therapies. For example, dendritic cells can be co-cultured with T lymphocytes and protein antigen in vitro, thus driving the ex vivo antigen-specific activation of T cells. The activated T cells are then administered autologously to effect an antigen-specific immune response in vivo (WO 97/24438). In another example, T cells can be-activated in vitro by contacting the T lymphocytes with dendritic cells that directly express an antigenic protein from a recombinant construct. The activated T cells can be used for autologous infusion (WO 97/29183).

T cells activated with specific peptides or protein fragments become immunizing agents against the proteins, cells or organisms from which the peptides or fragments were derived. For example, dendritic cells may be loaded with tumor-specific peptides. Specific application of DC-driven ex vivo T cell activation to the treatment of prostate cancer is described and claimed in U.S. Pat. No. 5,788,963. Mayordomo et al. demonstrated bone marrow-derived dendritic cells pulsed with synthetic tumor peptides elicit protective and therapeutic anti-tumor immunity (Nature Medicine 1:1297-1302 (1995); J. Exp. Med., 183:1357-1365 (1996)). The U.S. Pat. No. 5,698,679 describes immunoglobulin fusion proteins that deliver antigenic peptides to targeted antigen presenting cells (APCs), including dendritic cells, in vivo. This same approach may be used with peptides or antigens derived from viruses, bacteria, or parasites to create viral, bacterial, or parasitic vaccines.

Dendritic cells are also targets for therapeutic intervention in the treatment of various immune-mediated disorders. For example, dendritic cells have been implicated as an important player in the pathogenesis and pathophysiology of AIDS (e.g., serve as reservoirs for the HIV virus). See Zoeteweij et al., J. Biomed. Sci. 5(4):253-259 (1998); Grouard et al., Curr. Opin. Immunol. 9(4):563-567 (1997); Weissman et al., Clin. Microbiol. Rev. 10(2):358-367 (1997). In vitro methods for screening pharmaceutical candidates for agents that abrogate HIV infection of DC are described in U.S. Pat. No. 5,627,025. In another example, dendritic cells can be manipulated to induce T cell unresponsiveness to donor tissue or organ in a recipient (see U.S. Pat. No. 6,375,950).

Granulocytes can be used in granulocyte transfusions in the treatment or prevention of infections, e.g., bacterial neonatal sepsis, neutropenia-associated infections in cancer patients, and potential infections in patients receiving bone-marrow transplants. Granulocytes can also be used in prevention or treatment of allergy. For example, granulocytes involved in IgE-mediated inflammation (i.e., granulocytes coated with IgE antibodies some of which having specificity for the allergen) can be inactivated and used to alleviate the symptoms of an already established immune response against the allergen (see U.S. Pat. No. 6,383,489).

Thus, in one embodiment of the invention, a population of granulocytes in an individual is expanded from the progenitor cells of the invention by a method comprising administering to said individual a therapeutically-effective amount of a compound of the invention, wherein said amount is sufficient to induce the production of a plurality of granulocytes from CD34⁺ cells endogenous to said individual. In another embodiment, a population of granulocytes is expanded within an individual by a method comprising administering to said individual a population of CD34⁺ or CD133⁺ progenitor cells, wherein said cells have been contacted with a compound of the invention for at least three days, and administering said population of cells to said individual. In another embodiment, population of granulocytes is expanded within an individual by a method comprising administering to said individual a population of CD34⁺ or CD133⁺ progenitor cells and a compound of the invention, wherein the dose of said compound of the invention is sufficient to cause differentiation of a plurality of said population of cell into granulocytes. In a specific embodiment of the above embodiments, said CD34⁺ progenitor cells are CD34⁺CD38⁻CD33⁺ cells.

4.6.5. Treatment of Other Diseases and Conditions

The differentiated stem and progenitor cells of the invention, or the compounds of the invention, may also be used, alone or in combination, to treat or prevent a variety of other diseases or conditions. In certain embodiments, for example, the disease or disorder includes, but is not limited to, but not limited to a vascular or cardiovascular disease, atherosclerosis, diabetes, aplastic anemia, myelodysplasia, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis (ALS), ischemic renal disease, brain or spinal cord trauma, heart-lung bypass, glaucoma, retinal ischemia, retinal trauma, lysosomal storage diseases, such as Tay-Sachs, Niemann-Pick, Fabry's, Gaucher's, Hunter's, and Hurler's syndromes, as well as other gangliosidoses, mucopolysaccharidoses, glycogenoses, inborn errors of metabolism, adrenoleukodystrophy, cystic fibrosis, glycogen storage disease, hypothyroidism, sickle cell anemia, Pearson syndrome, Pompe's disease, phenylketonuria PKU), porphyrias, maple syrup urine disease, homocystinuria, mucoplysaccharidosis, chronic granulomatous disease and tyrosinemia, Tay-Sachs disease, cancer, tumors or other pathological or neoplastic conditions.

In other embodiments, the cells of the invention (e.g., which have been exposed to the compounds of the invention) may be used in the treatment of any kind of injury due to trauma, particularly trauma involving inflammation. Examples of such trauma-related conditions include central nervous system (CNS) injuries, including injuries to the brain, spinal cord, or tissue surrounding the CNS injuries to the peripheral nervous system (PNS); or injuries to any other part of the body. Such trauma may be caused by accident, or may be a normal or abnormal outcome of a medical procedure such as surgery or angioplasty. The trauma may be related to a rupture or occlusion of a blood vessel, for example, in stroke or phlebitis. In specific embodiments, the cells may be used in autologous or heterologous tissue regeneration or replacement therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues.

In a specific embodiment, the disease or disorder is aplastic anemia, myelodysplasia, leukemia, a bone marrow disorder or a hematopoietic disease or disorder. In another specific embodiment, the subject is a human.

4.7. Pharmaceutical Compositions

The present invention encompasses pharmaceutical compositions comprising a dose and/or doses of one or more of the compounds of the invention, wherein said dose or doses are effective upon single or multiple administration, prior to or following transplantation of conditioned or unconditioned human CD34⁺ or CD133⁺ progenitor or stem cells to an individual, exerting effect sufficient to inhibit, modulate and/or regulate the differentiation of these stem and/or progenitor cells into specific cell types, e.g., hematopoietic lineage cells, particularly myeloid lineage cells. In this context, as elsewhere in the context of this invention, “individual” means any individual to which the compounds or cells are administered, e.g., a mammal, bird or reptile.

Thus, in a specific embodiment, said dose or doses of the compounds of the invention, administered to an individual, modulate the differentiation of endogenous CD34⁺ progenitor cells into dendritic cells. In a more specific embodiment, the dose or doses increase the number of granulocytic cells in said individual to which said dose or doses have been administered. In another more specific embodiment, the dose or doses increase the number of CD34⁺CD38⁻CD33⁺ or CD34⁺CD38⁻CD33⁻ progenitor cells in a mammal to which said dose or doses have been administered.

In other embodiments, CD34⁺ or CD133⁺ progenitor or stem cells of interest are transplanted into human subject or patient in need thereof. Subsequent to transplantation, a compound of the invention is administered to the human subject or patient, to modulate the differentiation of the transplanted cells of interest in vivo. In a specific embodiment, such cells are differentiated in vivo into granulocytes. In yet other embodiments, the differentiation of progenitor or stem cells of interest in a human subject or patient is modulated in situ by administration of a compound of the invention.

In yet another embodiment, the invention provides pharmaceutical compositions comprising isolated cord blood stem or progenitor cell populations that have been augmented with hematopoietic progenitor cells that have been differentiated by exposure to compounds that inhibit PDE IV activity, in accordance with the methods of the invention. In another embodiment, the invention provides pharmaceutical compositions comprising cord blood that is supplemented with stem or progenitor cells contacted with the compounds of the invention; in a specific embodiment, said stem or progenitor cells have been differentiated by said compounds.

In yet another embodiment, the invention provides for pharmaceutical compositions comprising both one or more of the PDE IV inhibitors of the invention, and the stem and/or progenitor cells of the invention. Such compositions may be prepared 1, 2, 3, 4, 5, 6, 7, 8, 9, 10, 11 or 12 days in advance of administration so as to modulate the differentiation of the stem and/or progenitor cells along different developmental/differentiation pathways.

In yet another embodiment, the pharmaceutical compositions of the present invention may comprise the stem or progenitor cells themselves, wherein said cells have been differentiated according to the methods disclosed herein. Thus, the present invention provides a pharmaceutical composition comprising a plurality of stem cells and/or progenitor cells, wherein said plurality of stem and/or progenitor cells has been contacted with one or more of the PDE IV inhibitors of the invention in a concentration and for a duration sufficient for said compound(s) to modulate differentiation of said cells.

Thus, the pharmaceutical compositions of the invention comprise the compounds of the invention, administered to an individual; the cells of the invention, administered to an individual, in combination with the compounds of the invention, separately administered; and the cells of the invention, contacted with the compounds of the invention, administered to said individual.

The invention provides methods of treatment and prevention of a disease or disorder by administration of a therapeutically effective amount of a compound or a composition of the invention to a mammalian, preferably human, subject, in order to effect modulation of the proliferation and/or differentiation of CD34⁺ or CD133⁺ progenitor cells or stem cells transplanted to, or residing within the subject. In one embodiment, the invention provides a method of modulating the differentiation of CD34⁺ and CD133⁺ progenitor or stem cells so as to increase within a mammal the number of granulocytic cells. In another embodiment, any cell lineage that may be derived from a CD34⁺ and/or CD133⁺ progenitor or stem cell may be modulated by administration of the compounds of the invention to a mammal, preferably to a human. The term “mammal” as used herein, encompasses any mammal. Preferably a mammal is in need of such treatment or prevention. Examples of mammals include, but are not limited to, cows, horses, sheep, pigs, cats, dogs, mice, rats, rabbits, guinea pigs, monkeys, etc., more preferably, a human.

Administration of compounds of the invention can be systemic or local. In most instances, administration to a mammal will result in systemic release of the compounds of the invention (i.e., into the bloodstream). Methods of administration include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, intraocular and intracameral injection and non-injection routes, such as intravaginal rectal., or nasal administration. Preferably, the compounds and compositions of the invention are administered orally. In specific embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The compounds of the invention can be administered via typical as well as non-standard delivery systems, e.g., encapsulation in liposomes, midroparticles, microcapsules, capsules, etc. For example, the compounds and compositions of the invention can be delivered in a vesicle, in particular a liposome (see Langer, 1990, Science 249:1527-1533; Treat et al in Liposomes in Therapy of Infectious Disease and Cancer, Lopez-Berestein and Fidler (eds.), Liss, New York, pp. 353-365 (1989); Lopez-Berestein, ibid., pp. 317-327; see generally ibid.). In another example, the compounds and compositions of the invention can be delivered in a controlled release system. In one embodiment, a pump may be used (see Langer, supra; Sefton, 1987, CRC Crit. Ref Biomed. Eng. 14:201; Buchwald et al., 1980, Surgery 88:507 Saudek et al., 1989, N. Engl. J. Med. 3:574). In another example, polymeric materials can be used (see Medical Applications of Controlled Release, Langer and Wise (eds.), CRC Press., Boca Raton, Fla. (1974); Controlled Drug Bioavailability, Drug Product Design and Performance, Smolen and Ball (eds.), Wiley, New York (1984); Ranger and Peppas, 1983,J. Macromol. Sci. Rev. Macromol. Chem. 23:61; see also Levy et al., 1985, Science 228:190; During et al., 1989, Ann. Neurol. 25:351; Howard et al., 1989, J: Neurosurg. 71:105). In still another example, a controlled-release system can be placed in proximity of the target area to be treated, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) can be used. When administered as a composition, a compound of the invention will be formulated with a suitable amount of a pharmaceutically acceptable vehicle or carrier so as to provide the form for proper administration to the mammal. The term “pharmaceutically acceptable” means approved by a regulatory agency of the Federal or a state government or listed in the U.S. Pharmacopeia or other generally recognized pharmacopeia for use in mammals, and more particularly in humans. The term “vehicle” refers to a diluent, adjuvant, excipient, or carrier with which a compound of the invention is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gun acacia, gelatin, starch paste, talc, keratin, colloidal silica, urea, and the like. In addition, auxiliary, stabilizing, thickening, lubricating and coloring agents may be used. Preferably, when administered to a mammal, the compounds and compositions of the invention and pharmaceutically acceptable vehicles, excipients, or diluents are sterile. An aqueous medium is a preferred vehicle when the compound of the invention is administered intravenously, such as water, saline solutions, and aqueous dextrose and glycerol solutions.

The present compounds and compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In a preferred embodiment, the compounds and compositions of the invention are formulated for administration in accordance with routine procedures as a pharmaceutical composition adapted for oral or intravenous administration to humans. In one embodiment, the pharmaceutically acceptable vehicle is a hard gelatin capsule. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

Compounds and compositions of the invention formulated for oral delivery, are preferably in the form of capsules, tablets, pills, or any compressed pharmaceutical form. Moreover, where in tablet or pill form, the compounds and compositions may be coated to delay disintegration and absorption in the gastrointestinal tract thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound that swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles, excipients, and diluents, such as magnesium stearate, sodium saccharine, cellulose, magnesium carbonate, lactose, dextrose, sucrose, sorbitol, mannitol, starch, gum acacia, calcium silicate, microcrystalline cellulose, polyvinylpyrrolidone, water, syrup, and methyl cellulose, the formulations can additionally include lubricating agents, such as talc, magnesium stearate, mineral oil, wetting agents, emulsifying and suspending agents, preserving agents such as methyl- and propylhydroxybenzoates. Such vehicles are preferably of pharmaceutical grade. Orally administered compounds and compositions of the invention can optionally include one or more sweetening agents, such as fructose, aspartame or saccharin; one or more flavoring agents such as peppermint, oil of wintergreen, or cherry; or one or more coloring agents to provide a pharmaceutically palatable preparation.

A therapeutically effective dosage regimen for the treatment of a particular disorder or condition will depend on its nature and severity, and can be determined by standard clinical techniques according to the judgment of a medical practitioner. In addition, in vitro or in vivo assays can be used to help identify optimal dosages. Of course, the amount of a compound of the invention that constitutes a therapeutically effective dose also depends on the administration route. In general, suitable dosage ranges for oral administration are about 0.001 milligrams to about 20 milligrams of a compound of the invention per kilogram body weight per day, preferably, about 0.7 milligrams to about 6 milligrams, more preferably, about 1.5 milligrams to about 4.5 milligrams. In a preferred embodiment, a mammal, preferably, a human is orally administered about 0.01 mg to about 1000 mg of a compound of the invention per day, more preferably, about 0.1 mg to about 300 mg per day, or about 1 mg to about 250 mg in single or divided doses. The dosage amounts described herein refer to total amounts administered; that is, if more than one compound of the invention is administered, the preferred dosages correspond to the total amount of the compounds of the invention administered. Oral compositions preferably contain 10% to 95% of a compound of the invention by weight. Preferred unit oral-dosage forms include pills, tablets, and capsules, more preferably capsules. Typically such unit-dosage forms will contain about 0.01 mg, 0.1 mg, 1 mg, 5 mg, 10 mg, 15 mg, 20 mg, 50 mg, 100 mg, 250 mg, or 500 mg of a compound of the invention, preferably, from about 5 mg to about 200 mg of compound per unit dosage.

In another embodiment, the compounds and compositions of the invention can be administered parenterally (e.g., by intramuscular, intrathecal, intravenous, and intraarterial routes), preferably, intravenously. Typically, compounds and compositions of the invention for intravenous administration are solutions in sterile isotonic aqueous vehicles, such as water, saline, Ringer's solution, or dextrose solution. Where necessary, the compositions may also include a solubilizing agent. Compositions for intravenous administration may optionally include a local anesthetic such as lignocaine to ease pain at the site of the injection. For intravenous administration, the compounds and compositions of the invention can be supplied as a sterile, dry lyophilized powder or water-free concentrate in a hermetically sealed container, such as an ampule or sachette, the container indicating the quantity of active agent. Such a powder or concentrate is then diluted with an appropriate aqueous medium prior to intravenous administration. An ampule of sterile water, saline solution, or other appropriate aqueous medium can be provided with the powder or concentrate for dilution prior to administration. Or the compositions can be supplied in pre-mixed form, ready for administration. Where a compound or composition of the invention is to be administered by intravenous infusion, it can be dispensed, for example, with an infusion bottle containing sterile pharmaceutical-grade water, saline, or other suitable medium.

Rectal administration can be effected through the use of suppositories formulated from conventional carriers such as cocoa butter, modified vegetable oils, and other fatty bases. Suppositories can be formulated by well-known methods using well-known formulations, for example see Remington: The Science and Practice of Pharmacy, Alfonso R. Gennaro ed., Mack Publishing Co. Easton, Pa., 19th ed., 1995, pp. 1591-1597, incorporated herein by reference.

To formulate and administer topical dosage forms, well-known transdermal and intradermal delivery mediums such as lotions, creams, and ointments and transdermal delivery devices such as patches can be used (Ghosh, T. K.; Pfister, W. R.; Yum, S. I. Transdermal and Topical Drug Delivery Systems, Interpharm Press, Inc. p. 249-297, incorporated herein by reference). For example, a reservoir type patch design can comprise a backing film coated with an adhesive, and a reservoir compartment comprising a compound or composition of the invention, that is separated from the skin by a semipermeable membrane (e.g., U.S. Pat. No. 4,615,699, incorporated herein by reference). The adhesive coated backing layer extends around the reservoir's boundaries to provide a concentric seal with the skin and hold the reservoir adjacent to the skin.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In one embodiment, the kit contains more than one compound of the invention. In another embodiment, the kit comprises a compound of the invention and another biologically active agent.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred. The compounds and compositions of the invention may also be demonstrated to be effective and safe using animal model systems. Other methods will be known to the skilled artisan and are within the scope of the invention.

4.8. Assays Using the Methods of the Invention

The methodology described above, i.e., the examination of the effect of SelCIDs on the differentiation on early progenitor cells, such as CD34⁺ cells, can be applied to any compound of interest, the effect of which on differentiation is desired to be known. This may be accomplished in several ways.

In one embodiment, the compound may simply substitute for a SelCID or any of the other compounds of the invention. Here, CD34⁺ progenitor cells and/or Cb133⁺ progenitor cells may be contacted with the compound of interest, at varying concentrations, under conditions that allow for the proliferation and/or differentiation of the progenitor cells into committed and/or fully-differentiated cells. The culture methods disclosed herein, particularly the culture methods disclosed in Section 4.4, may be used. The effect, if any, of the compound of interest is determined by assessing the change, if any, in the cell populations that differentiate from the progenitor cells, where the change may be monitored by any phenotypic change, but is preferably assessed by determining cell surface markers that are present or absent. Like the methods of the invention, the compound of interest may be administered in a single dose at any time from initial culture to achievement of the finally-differentiated cell(s). Alternatively, the compound of interest may be administered in multiple doses during the proliferative stage, the differentiation stage, or both. The change in phenotypic characteristics of the proliferating/differentiating progenitor cells is preferably compared to a control culture of cells, such as DMSO-treated cells. Of particular interest would be any effects on proliferation or differentiation such as, but not limited to: modulation of the rate of proliferation; modulation of the rate of differentiation; modulation of differentiation of the progenitor cells into specific committed precursor cells; blocking the differentiation into particular cell types; and enhancing the differentiation into particular cell types.

In another embodiment, culturing, proliferation and differentiation takes place as above, but the compound of interest is contacted with the progenitor cell(s) along with a PDE IV inhibitor, such as a SelCID™. In this manner, the effects, possibly synergistic, of multiple compounds may be determined. Of particular interest would be any compounds that have no, or a slight, effect on proliferation or differentiation alone, but have a significant effect in combination with a SelCID™ or prodrug thereof. In another embodiment, any two compounds of interest may be contacted with the progenitor cells under culture conditions, as above, that normally allow for the proliferation and differentiation of the progenitor cells. Here, preferably an experiment in which precursor cells are contacted with two compounds of interest contains a control in which the progenitor cells are contacted with only one of each of said compounds; a control in which the cells are contacted with a PDE IV inhibitor, such as a SelCID™; and a control in which cells are not contacted with a compound, or are contacted with DMSO. Again, the variations in the dosages, and timing of dosing, are as described above and in Section 4.4.

### 5. WORKING EXAMPLES

### 5.1. Example 1

### Effects of PDE IV Inhibitors on Differentiation of CD34+ Progenitor Cells

The following assay is utilized to determine the effects of PDE IV inhibitors on the differentiation of CD34+ (hematopoietic progenitor) cells and the generation of colony forming units (CFU). Significantly, the assay demonstrates the ability of PDE IV inhibitors to suppress specifically the generation of erythropoietic colonies (BFU-E and CFU-E), while augmenting both the generation of leukocyte and platelet forming colonies (CFU-GM) and enhancing total colony forming unit (CFU-Total) production. The methods of the invention can therefore be used to regulate the differentiation of stem cells, and can also be used to stimulate the rate of colony formation, providing significant benefits to hematopoietic stem cell transplantation by improving the speed of bone marrow engraftment and recovery of leukocyte and/or platelet production.

Cord blood CD34⁺ hematopoietic progenitor cells are plated in 96 well cultivation dishes at a density of 1000 cells per well in IMDM supplemented with 20% fetal calf serum and cytokines (IL-3, G-CSF and kit-ligand (R&D Systems, Inc.). The cells are exposed to one or more PDE IV inhibitors, or DMSO (a control compound), and allowed to culture for 6 days. Cord blood CD34+ cells are plated in 96 well cultivation dishes at a density of 1000 cells per well in IMDM supplemented with 20% fetal calf serum and cytokines (IL-3, G-CSF and kit-ligand (KL) (R&D Systems, Inc.)). After culturing, cells are stained and sorted with a fluorescence activated cell sorter (FACS). 400 μL of stained cells are harvested and diluted to 1.0 ml with 1% fetal calf serum in phosphate buffered saline (PBS). Cells are counted to determine the effect of modulation of stem cell differentiation. Results will show suppression of generation of red blood cells or erythropoietic colonies (BFU-E and CFU-E), augmentation of the generation of both leukocyte and platelet forming colonies (CFU-GM), and enhancement total colony forming unit production. The methods of the invention can therefore be used to regulate the differentiation of stem cells, and can also be used to stimulate the rate of specific colony formation, providing significant benefits to hematopoietic stem cell transplantation by improving the speed of bone marrow engraftment and recovery of leukocyte and/or platelet production by origin stem cell commitment toward desired engraftable lineages.

### 5.2. Example 2

### Effects of PDE IV Inhibitors on Differentiation of Human Cord Blood CD34⁺ Progenitor Cells

In the following example, the effect of PDE IV inhibitors on the proliferation and differentiation of cord blood (CB) mononuclear cells into CD34+ (hematopoietic progenitor) cells is studied. Cord blood mononuclear cells are a mixed population of cells including a small population of hematopoietic progenitor (CD34+) cells. A subset of this small CD34+ cell population includes a population (approximately 1% of total CB mononuclear cells) of CD34+CD38+ cells and an even smaller population (less than 1% of total CB mononuclear cells) of CD34+CD38− cells. Significantly, PDE IV inhibitors will cause an up-regulation (increased differentiation) of CD34+ cells, and inhibition or slowing down of the differentiation of hematopoietic stem cells or progenitor cells compared with positive and negative controls.

5.2.1. Materials and Methods

CB CD34+ cells are initiated at 4×10⁴ cells/ml in a 24-well plate in 20% FCS IMDM (fetal calf serum/Iscove's Modified Dulbecco's Medium) supplemented with cytokines (IL3, G-CSF and Kit-ligand) (R&D Systems, Inc.). PDE IV inhibitors are included in the culture at various concentrations. The same volumes of DMSO are used as controls. A negative control without any compound is also used. Cells are cultured at 37° C. and 5% CO₂ in a humidified incubator for 7 days. Cells are then harvested from each well.

The total cell number from each well is determined by counting in a CELL-DYN® 1700 (Abbott Diagnostics) and the expression of CXCR4, CD45, CD34, CD38, CD11b and Gly-A expression is analyzed by FACS (fluorescence-activated cell sorting) staining.

### 5.3. Example 3

### Effect of PDE IV Inhibitors on Human Cord Blood MNC Cells

Cord blood MNCs that have been cryopreserved and thawed using standard methods are isolated by standard Ficoll separation method and cultured in 24 well-plate at 0.5×10⁶ cells/ml in 20% FCS-IMDM with cytokines (IL6, KL and G-CSF 10 ng/ml each) in triplicate. The experimental groups are None (cytokines only), DMSO (1.7 ul), and varying concentrations of a PDE IV inhibitor in DMSO. The cultured cells are harvested and analyzed by FACS staining after 1 week of culture.

### 5.4. Example 4

### Effects of PDE IV Inhibitors on Monocyte Production

Purified human cord blood CD34⁺ cells (greater than 90% CD34⁺) are cultured in 20% FCS IMDM medium supplemented with cytokines (IL3, IL6, G-CSF, KL and Epo) at 4×104 cells/ml for 14 days at 37° C. in a humidified 5% CO2 incubator. The experimental groups consist of a group in which (i) no DMSO or chemical compounds were added (“None”), (ii) DMSO only, and (iii) a PDE IV inhibitor dissolved in DMSO. Aliquots of cells are harvested and stained with CD34-PE conjugated monoclonal antibody and CD14-FITC conjugated monoclonal antibody.

### 5.5. Example 5

### Effects of PDE IV Inhibitors on Transplanted Nucleated Cells from Umbilical Cord Blood and Placenta

This experiment demonstrates that PDE IV inhibitor pre-treatment increases the survival of transplanted placental nucleated cells (PLNC), umbilical cord blood nucleated cells (UCBNC) and bone marrow cells (BMNC).

Placental nucleated cells (PLNC), umbilical cord blood nucleated cells (UCBNC) and bone marrow cells (BMNC) are obtained from human donors. PLNC and UCBNC are obtained from placenta and umbilical cord.

The cells are pretreated by incubating them in DMEM supplemented with 2% human CB serum with 10 μg/ml of a PDE IV inhibitor for 24 hours. Cells are then washed, resuspended in autologous plasma and administered intravenously to recipient adult SJL/L mice (Jackson Laboratories) that have had bone marrow ablation produced by lethal irradiation (900 cGy) according to standard methods. Such irradiation is better than 90% lethal by 50 days post-irradiation (Ende et al., 2001, Life Sciences 69(13):1531-1539; Chen and Ende, 2000, J. Med. 31: 21-30; Ende et al., 2000, Life Sci. 67(1):53-9; Ende and Chen, 2000, Am. J. Clin. Pathol. 114: 89).

### 5.6. Example 6

### Effects of SelCIDS™ on Differentiation of CD34+ Progenitor Cells

The following example analyzes the effects of SelCIDs™ on the differentiation of CD34⁺ hematopoietic progenitor) cells and the generation of colony forming units (CFU). Significantly, the results demonstrate that SelCIDs™ can be used to suppress specifically the generation of erythropoietic colonies (BFU-E and CFU-E), while augmenting both the generation of leukocyte and platelet forming colonies (CFU-GM) and enhancing total colony forming unit (CFU-Total) production.

The methods of the invention can therefore be used to regulate the differentiation of stem cells, and can also be used to stimulate the rate of colony formation, providing significant benefits to hematopoietic stem cell transplantation by improving the speed of bone marrow engraftment and recovery of leukocyte and/or platelet production.

Cord blood CD34⁺ hematopoietic progenitor cells are plated in 96 well cultivation dishes at a density of 1000 cells per well in IMDM supplemented with 20% fetal calf serum and cytokines (IL-3, GCSF and kit-ligand (R&D Systems, Inc.). The cells are exposed to SelCIDs™ or DMSO (a control compound) and allowed to culture for 6 days. Cord blood CD34+ cells are plated in 96 well cultivation dishes at a density of 1000 cells per well in IMDM supplemented with 20% fetal calf serum and cytokines (IL-3, GCSF and kit-ligand (KL) (R&D Systems, Inc.)). After culturing, cells are stained and sorted with a fluorescence activated cell sorter (FACS). 400 μL of stained cells are harvested and diluted to 1.0 ml with 1% fetal calf serum in phosphate buffered saline (PBS). Cells are counted to determine the effect of modulation of stem cell differentiation.

The compounds of the invention are effective in the modulation of the lineage commitment of hematopoietic progenitor stem cells. Thus, SelCIDs™ can be used to suppress specifically the generation of red blood cells or erythropoietic colonies (BFU-E and CFU-E), while augmenting both the generation of leukocyte and platelet forming colonies (CFU-GM) and enhancing total colony forming unit production. The methods of the invention can therefore be used to regulate the differentiation of stem cells, and can also be used to stimulate the rate of specific colony formation, providing significant benefits to hematopoietic stem cell transplantation by improving the speed of bone marrow engraftment and recovery of leukocyte and/or platelet production by origin stem cell commitment toward desired engraftable lineages.

### 5.7. Example 7

### Effects of SelCIDs™ on Proliferation and Differentiation of Human Cord Blood CD34⁺ Cells

In the following example, the effects of SelCIDs™ on the proliferation and differentiation of cord blood (CB) mononuclear cells into CD34+ (hematopoietic progenitor) cells are studied. Cord blood mononuclear cells are a mixed population of cells including a small population of hematopoietic progenitor (CD34+) cells. A subset of this small CD34⁺ cell population includes a population (approximately 1% of total CB mononuclear cells) of CD34+CD38+ cells and an even smaller population (less than 1% of total CB mononuclear cells) of CD34⁺CD38⁻ cells. SelCIDs™ causes an up-regulation (increased differentiation) of CD34+ cells, and can apparently inhibit or slow down the differentiation of hematopoietic stem cells or progenitor cells compared with the positive and negative controls.

5.7.1. Materials and Methods

CB CD34+ cells are initiated at 4×10⁴ cells/ml in a 24-well plate in 20% FCS IMDM (fetal calf serum/Iscove's Modified Dulbecco's Medium) supplemented with cytokines (1L3, GCSF and Kit-ligand) (R&D Systems, Inc.). SelCIDs™ is included in the culture at three different concentrations: 5 μg/ml, 1 μg/ml and 0.3 μg/ml. The same volumes of DMSO are used as controls. A negative control without any compound is also used. Cells are cultured at 37° C. and 5% CO₂ in a humidified incubator for 7 days. Cells are then harvested from each well.

The total cell number from each well is determined by counting in a CELL-DYN® 1700 (Abbott Diagnostics) and the expression of CXCR4, CD45, CD34, CD38, CD1 lb and Gly-A expression is analyzed by FACS (fluorescence-activated cell sorting) staining.

CB cells from two different donors (CB2276 and CB2417) are cultured, assayed and analyzed separately.

5.7.2. Results and Discussion

The effects of SelCIDs™ on cytokine-stimulated expansion of CD34+ cells is tested. SelCIDs™ does not have a significant effect on the proliferation of CD34+ cells that are cultured in the presence of IL-3, Kit-ligand (KL) and G-CSF when compared with the negative control. However, SelCIDs™ are expected to induce a yield of a higher number of cells, when compared with the DMSO control.

The effects of SelCIDs™ on expression of cell differentiation are analyzed by FACS analysis of surface proteins CXCR4 and CD34. SelCIDs™ are expected to show an inhibitory effect upon the expression of CXCR4.

With respect to surface protein CD34⁺, SelCIDs™ are expected to cause up-regulation (increased proliferation) of CD34⁺ cells. In SelCID™ treated cells, the majority of CD34⁺ and CD34⁻ cells will be CD38⁻, while cells in the control and DMSO-treated populations will mainly be CD38+. This indicates that SelCIDs™ can be used to suppress specifically the generation of red blood cells or erythropoietic colonies (BFU-E and CFU-E), while augmenting both the generation of leukocyte and platelet forming colonies (CFU-GM) and enhancing total colony forming unit production. The methods of the invention can therefore be used to regulate the differentiation of stem cells, and can also be used to stimulate the rate of colony formation, providing significant benefits to hematopoietic stem cell transplantation by improving the speed of bone marrow engraftment and recovery of leukocyte; and/or platelet production.

The effect of SelCIDS™ on expression of cell differentiation is analyzed by FACS analysis of surface proteins of cells that are CD34+CD38+ versus CD34+CD38+ or that are CD11b+. The level of CD11b expression is decreased in SelCIDs™-treated cells, as determined by mean immunofluorescence (MIF), indicating that CD11b expression is repressed. CD11b+ cells are therefore at a less differentiated state when cultured in the presence of SelCIDs™.

### 5.8. Example 8

### Effects of SelCIDs™ on Human Cord Blood MNC Cells

In the previous examples, SelCIDs™ are expected to significantly down-regulate the expression of CXCR4 in cord blood CD34+ cells and to increase the CD34+CD38− cell population. E this example, SelCIDs™ are shown to have similar activities on cord blood mononucleated cells (MNC).

Cord blood MNCs that have been cryopreserved and thawed using standard methods are isolated by standard Ficoll separation method and cultured in 24 well-plate at 0.5×10⁶ cells/1 ml in 20% FCS-IMDM with cytokines (IL6, KL and G-CSF 10 ng/ml each) in triplicates. The experimental groups are None (cytokines only), DMSO (1.7 μl), SelCIDs™ (5.0 μg in 1.7 μl DMSO). The cultured cells are harvested and analyzed by FACS staining after 1 week of culture.

The total cell numbers of MNCs cultured with DMSO, SelCIDs™ are expected to be lower than in the control group (“None,” cytokines only). Cell cultures that are cultured with MID 1 should exhibit a higher percentage of CD34+ cells than all the other groups, while the total numbers of CD34+ cells should be similar in all groups. Numbers of CD34+CD38− cells will be significantly higher in SelCIDs™ treated cells, which is consistent with the results of treating purified CD34+ cells with the compounds. It is well accepted that CD34+CD38− cells are a less differentiated hematopoietic progenitor cell which engrafts and proliferates after transplantation at a higher efficiency than CD34+CD38+ cells (Dao et al. 1998, Blood 91 (4): 1243-55; Huang et al., 1994, Blood 83(6): 1515-26).

A majority of CXCR4+ cells in the cultures of SelCIDs™-treated cells are CD45 negative. This cell population is significantly higher in the SelCIDs™ treated cells.

The results indicate that SelCIDs™ is useful in conditioning stem cells to counteract the deleterious effects of cryopreservation, thawing and/or exposure to cryopreservatives on stem cells. The results further indicate that the suppression by DMSO of CD34+ and CD 14+ cell production can counteracted by treating with SelCIDs™, which enhances that proliferative capacity of CD34+ and CD14+ cells.

### 5.9. Example 9

### Effects of SelCIDs™ on Monocyte Production

Purified human cord blood CD34+ cells (greater than 90% CD34+) are cultured in 20% FCS IMDM medium supplemented with cytokines (IL3, IL6, G-CSF, KL and Epo) at 4×10⁴ cells/ml for 14 days at 37° C. in a humidified 5% CO₂ incubator. The experimental groups consist of a group in which (i) no DMSO or chemical compounds are added (“None”), (ii) DMSO only, (iii) SelCIDs™ dissolved in DMSO. Aliquots of cells are harvested and stained with CD34−PE conjugated monoclonal antibody and CD14-FITC conjugated monoclonal antibody. The SelCIDs™-treated group is expected to show a significantly higher percentage of CD34+ cells than the control groups, Moreover, the production of monocytes decreases, as evidenced by a drop in the number of CD14⁺ cells. Since the SelCIDs™ treated groups are exposed to DMSO as well, it can be deduced that the monocyte production that is inhibited-by DMSO is overcome by treatment with SelCIDs™.

### 5.10. Example 10

### Effects of SelCIDs™ Pretreatment on Transplanted Nucleated Cells from Umbilical Cord Blood and Placenta

This experiment demonstrates that SelCIDs™ pre-treatment increases the survival of transplanted placental nucleated cells (PLNC), umbilical cord blood nucleated cells (UCBNC) and bone marrow cells (BMNC).

Placental nucleated cells (PLNC), umbilical cord blood nucleated cells (UCBNC) and bone marrow cells (BMNC) are obtained from human donors. PLNC and UCBNC are obtained from placenta and umbilical cord.

The cells are pretreated by incubation in DMEM supplemented with 2% human CB serum with 10 g/ml SelCIDs™ for 24 hours. Cells are then washed, resuspended in autologous plasma, and administered intravenously to recipient adult SJL/L mice (Jackson Laboratories) that have bone marrow ablation produced by lethal irradiation (900 cGy) according to standard methods. Such irradiation is better than 90% lethal by 50 days post-irradiation (Ende et al., 2001, Life Sciences 69(13):1531-1539; Chen and Ende, 2000, J. Med. 31: 21-30; Ende et al., 2000, Life Sci. 67(1):53-9; Ende and Chen, 2000, Am. J. Clin. Pathol. 114: 89).

SelCIDs™ pre-treatment increases the survival of transplanted placental nucleated cells (PLNC), umbilical cord blood nucleated cells (UCBNC) and bone marrow cells (BMNC).

### 5.11. EXAMPLE 11

### Modulation of Differentiation of CD34⁺ Progenitor Cells

Bone marrow and cord blood CD34⁺ progenitor cells are obtained from Clonetics and cultured in Iscove's MDM with BIT 95000 (StemCell Technologies) in the presence of SCF, Flt-3L, GM-CSF and TNF-α for 6 days, and then in the presence of GM-CSF and TNF-α for 6 additional days.

Analysis of cell surface phenotype: Cells are processed for double staining (30 min at 4° C.) at day 6 and day 12 using FITC and PE conjugated mAbs. Antibodies used are from BD Pharmingen: CD34 (PE), CD38 (FITC), CD33 (FITC), CD1a (FITC), CD86 (PE), CD14 (PE), CD83 (PE), CD54 (E), CD11b (PE), CD11c (PE), HLA-DR (PE), CD15 (FITC), and CD133 (PE) from Miltenyi. Fluorescence analysis is performed on a FACScan flow cytometer after acquisition of 10,000 events (Coulter).

Detection of apoptosis: Phosphatidyl serine exposure is determined using Annexin V-FITC staining in combination with propidium iodide (BD Pharmingen apoptosis detection kit I) following manufacturer instruction.

Phagocytosis: The endocytic activity of the cells is analyzed by measuring FITC-dextran uptake. Cells are incubated with 1 mg/ml dextran-FITC (Sigma) in complete medium at 37° C. for 1 hour and 4° C. for 1 hour as a negative control.

T cell proliferation assay: After 13 days of culture, CD34⁺-derived DC cells are collected, and after treatment with mitomycin C (50 μg/l ml, Sigma), used as stimulators cells for allogenic adult CD3⁺ T cells purified from peripheral blood mononuclear cells PBMCs) from healthy volunteers. CD3⁺ T responder cells are used at a concentration of 5×10⁴ cells/well. Stimulators cells are added in graded doses to the T cells in black 96-well flat bottom, clear bottom tissue culture plates for chemiluminescence detection. Cultures are performed in RPMI 1640 medium supplemented with 10% heat-inactivated FBS, glutamine and Penicillin-streptomycin. After 6 days of culture, cell proliferation is measured with the BrdU chemiluminescence assay (Roche, Nutley N.J.), following manufacturer instructions. Results are presented as the mean± SD obtained from triplicate cultures.

SelCIDs™ can significantly alter the development of DC from CD34⁺ progenitors. To study the effect of SelCIDs™ on the generation of DC, CD34⁺ progenitors cells are cultured with or without SelCIDS™ (1 μM) for a period of 12 days during the expansion and maturation phase (day 1 to day 12), or a period of 6 days during the maturation phase (day 6 to day 12). The addition of SelCIDs™ from day 1 to day 12 is expected to inhibit the acquisition of the DC phenotype and more importantly increases the CD34⁺ CD38⁻ population, altering the normal differentiation of CD34⁺CD38⁻ cells into CD34⁺CD38⁺ cells. However, SelCID™ treated CD34⁺ cells are expected to acquire the CD33 myeloid marker, and these cells will present a CD34⁺CD38⁻CD33⁺ phenotype at day 6. SelCIDs™ can almost completely prevent the generation of CD1a⁺ cells at day 6, and particularly the generation of double positive CD86⁺CD1a⁺ cells. This double positive population is thought to be the precursor of epidermal Langerhans DC. SelCIDs™ can also decrease the generation of CD14⁺ CD1a⁻ cells that can give rise both to dermal DC and monocyte/macrophages. The increase in the early progenitor population (CD34⁺CD38⁻ cells) and the block in the myeloid DC progenitors (CD1a⁺CD14⁻ and CD1a⁻CD14⁺ cells) probably will be dose dependant and reached a maximum at 1 μM of SelCIDs™This effect is reversible and interference with the CD34 differentiation pathway is only observed if CD34⁺ progenitors are cultured for at least 3 days with SelCIDs™.

Multiple doses of SelCIDs™ between days 0 and 6 will intensify the increase in the CD34⁺ population.

CD34⁺ progenitor cells cultured in the presence of SelCIDs™ also displays at day 12 a decreased expression of co-stimulatory molecules (CD86, CD80). The CD54 adhesion molecule is altered with decreased expression of the CD₅₄^{bright} and increased expression of the CD54^{dim} populations. The expression of HLA-DR molecules is reduced in SelCIDs™ treated CD34⁺ progenitors.

When one or more SelCIDs™ is added at day 6, after culture from days 0-6 without treatment, and when the CD1a⁺ population has already been generated, SelCIDs™ increases the persistence of the CD1a⁺ population. The SelCIDs™ treated culture contains relatively more CD1a⁺ precursors at day 12 than the DMSO control. The addition of SelCIDs™ to day 6 CD34⁺ differentiated cells also decreases considerably the generation of CD14⁺ precursors and the expression of the co-stimulatory molecules (CD86, CD80).

SelCIDs™ promotes granulocytic differentiation: To determine if the block in DC generation is associated with a change to a different myeloid differentiation pathway, the expression of the CD15 granulocytic marker can be monitored. Expression of the CD15 surface molecule is increased in CD34⁺ progenitor cells cultured in the presence of SelCIDs™. In the presence of a cytokine cocktail that drives DC differentiation, the addition of SelCIDs™ diverts the expansion/maturation of progenitor cells into a more granulocyte-like phenotype. The skew in myeloid differentiation can be studied by monitoring the expression of 2 markers: CD11c, expressed by myeloid DC progenitors for Langerhans cells and interstitial DC, and CD15 expressed by granulocyte progenitors. A decrease in the CD11c+CD15− population is associated with a concomitant increase in the CD11c−CD 15⁺ granulocytic population. Interestingly, multiples doses of SelCIDs™ enhance the shift towards the granulocytic lineage.

Block in DC generation is not mediated by specific killing of the DC progenitors: To determine if the decrease in DC progenitors is mediated by specific killing, CD34⁺ progenitor cells are cultured for a period of 6 days in the presence of SCF, Flt-3L, GM-CSF and TNF-α. At day 6, CD1a⁺CD14⁻ and CD1a⁻CD14⁺ cells (DC progenitors) are isolated by magnetic cell sorting (Miltenyi). Purified populations are cultured for an additional 2 days in the presence of GM-CSF and TNF-α with or without SelCIDs™ (1 μM). There is no significant increase in the level of annexin V⁺- PI⁻(early apoptosis) and annexin V⁺-PI⁺ (late apoptosis) populations upon SelCIDs™ treatment.

Functional activity of DC generated from CD34⁺ progenitors is altered: The phagocytic capacity of cells derived from CD34⁺ progenitors cells cultured with cytokines with or without SelCIDs™ is assayed by the mannose receptor-mediated endocytosis of dextran-FITC at day 12. When one or more SelCIDs™ is added from day 1 to day 12, there is a strong decrease in the phagocytic capacity compared to DMSO control. When SelCIDs™ is added from day 6 to day 12 the phagocytic capacity is comparable to the DMSO-control cells.

The antigen presentation capacity (APC) of CD34⁺ cells cultured with cytokine with or without SelCIDs™ is evaluated by measuring their capacity to induce the proliferation of CD3⁺ allogenic T cells in a Mixed Leucocyte Reaction (MLR) assay at day 12. When SelCIDs™ is added from day 1 to day 12, the CD34⁺ cells show a reduced capacity to stimulate the proliferation of T-cells as compared to DMSO control. In contrast, when one or more SelCIDs™ is added from day 6 to day 12, the capacity to stimulate the proliferation of T-cells is comparable to the DMSO-control cells.

SelCIDs™ can dramatically attenuate the differentiation of CD34⁺ progenitor cells into dendritic cells. As a consequence, SelCID™-treated cells will present a low phagocytic capacity and a reduced APC capacity. SelCIDs™ will also increase early hematopoietic progenitors, the CD34⁺CD38⁻ cells. Those early hematopoietic progenitors have been shown to give better engraftment and repopulation in the NOD-SCID mouse model (Tamaki et al., J. Neurosci. Res. 69(6):976-86 (2002)). Moreover, SelCIDs™ skews CD34⁺ cells differentiation by switching myeloid differentiation toward the granulocytic lineage, even when the cytokine pressure is in favor of dendritic cell differentiation. In addition, SelCIDs™ is found to have no toxic effects on CD34⁺ cells, and not to impair the cells' ability to proliferate. This modulation of DC function and promotion of granulocytic differentiation can have significant therapeutic utility for the treatment of various cancers, immunological disorders, and infections diseases and in organ transplants, and regenerative medicine.

### 5.12. Example 12

### SelCIDs™ Modulates Differentiation of CD133⁺ Progenitor Cells

Multiple doses of SelCIDs™, in addition to intensifying the increase in the CD34⁺ population, also increases the expression of CD133, which is usually expressed by CD34^{bright} hematopoietic progenitor cells and some primitive CD34⁻ subpopulations. SelCIDs™, by enriching for the CD34⁺CD133⁺ primitive hematopoietic cells, should have clinical implication for hematopoietic recovery after stem cell transplantation. In addition, CD133⁺ stem cells can also give rise to the endothelial lineage and contribute in term to wound healing. Multiple doses of SelCIDs™ does not exacerbate the block in the generation of Langerhans DC precursors.

### 5.13. Example 13

### Generation of Murine Dendritic cells from Bone Marrow (BM) Sca⁺ Hematopoietic Progenitor Cells

Mouse bone marrow from inbred C57BL/6 mice are obtained from Clonetics. Hematopoietic Sca+Lin− progenitors are enriched using SpinSep murine progenitor enrichment cocktail (StemCell Technologies) and cultured in Iscove's MDM with BUI 95000 (StemCell Technologies) in the presence of murine growth factors SCF, Flt3L, GM-CSF and M-CSF for 9 days, to promote expansion of Sca+ cells and a DC precursor phenotype and then in the presence of GM-CSF and TNF-α for 3 additional days to drive the cells to an immature DC phenotype. Enriched Sca+Lin− cells are cultured in the presence of DMSO (0.1%), SelCIDs™ at 10 μM or all-trans retinoic acid (ATRA) (ICN Biomedicals) at 10 μM from day 0. Compounds are added to cells at day 0 and day 9.

Analysis of Murine cell surface phenotype: murine cells are processed for double staining (14 min at RT) at day 9 and day 12; using FITC and PE conjugated mAbs. Antibodies used are from BD Pharmingen: Sca (PE), CD11b (FITC), Gr-1 (ITC), CD86 (PE), CD14 (PE), CD80 (PE), I-Ab (PE), CD40 (PE) and CD32.1/16.1 (FITC) from Miltenyi. Fluorescence analysis is performed on a FACScan flow cytometer (Coulter) after acquisition of 10,000 events.

SelCIDs™ can alter the development of Murine DC from Sca⁺ progenitors. At day 9 cells will present a DC precursor phenotype with high surface expression of dendritic/myeloid markers CD32/16 (Fc receptors), CD11b, CD80, low expression of 1-A^{b} and CD86, and lack of expression of lineage markers as CD14 and Gr-1. SelCIDs™ will show no significant effect on cell surface marker expression by day 9, while ATRA will show marked downregulation of CD80, I-A^{b} and Sca+ expression (data not shown). However by day 12, SelCIDs™ may show downregulation of CD86 and bright I-A^{b} expression and upregulation of CD11b expression. ATRA shows similar but more pronounced effects than SelCIDs™. In addition, SelCIDs™ shows no effects on the expression of CD40 and CD80 while ATRA shows marked downregulation of these molecules.

SelCIDs™ inhibits the differentiation of DC precursors into immature DC by downregulating CD86 and MHC II expression. The compound's effects are not expected to be as dramatic as those observed in human hematopoietic progenitors. The effect of SelCIDs™ is much less pronounced than that of ATRA, which is a teratogen in mice.

### 5.14. Example 14

### Application of Differentiation Assay to Compounds other than SelCIDs

The methodology described above, ie., the examination of the effect of SelCIDs on the differentiation on early progenitor cells, such as CD34⁺ cells, can be applied to any compound of interest, the effect of which on differentiation is desired to be known. As an example of the extension of this assay method to other compounds, we compared the effect of retinoic acid (ATRA) and aspirin to that of SelCIDs™ on the differentiation of CD34⁺ cells toward the DC lineage versus the control (DMSO-treated) cells. Retinoic acid is studied because of its known effect on cellular proliferation and differentiation its therapeutic use in some cancer, and its known teratogenic effect. Conversely, the effect of aspirin is studied because it is a commonly-used anti-inflammatory drug with no immunomodulatory properties. The results at day 6 of CD34⁺ progenitors cells cultured in the presence of SCF, Flt-3L, GM-CSF and TNF-α, with or without compound for a period of 6 days can be obtained.

In the literature other drugs have been shown to modulate cellular differentiation, for example, a recent paper reports the modulation by corticosteroids of DC generation from CD34⁺ progenitors cells. The profile differs from SelCIDs™ with an increase in the CD1a+population and a decrease in the CD14⁺ population.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

### 6. LITERATURE

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A method for modulating the differentiation of a mammalian stem cell or progenitor cell comprising differentiating said stem cell or progenitor cell under suitable conditions and in the presence of a compound that inhibits PDE IV activity, wherein said compound is not a polypeptide, peptide, protein, hormone, cytokine, oligonucleotide, or nucleic acid.

2. The method of claim 1 wherein said stem cell is differentiated into a hematopoietic cell.

3. The method of claim 1 wherein said stem cell is selected from the group consisting of an embryonic stem cell, a placental stem cell, a cord blood stem cell, a peripheral blood stem cell, and a bone marrow stem cell.

4. The method of claim 1, wherein said PDE IV inhibitor is a SelCID™ or a prodrug thereof.

5. The method of claim 1 wherein said differentiating is conducted in cell culture.

6. The method of claim 1, wherein said differentiating is conducted within an individual.

7. The method of claim 1 wherein the concentration of the compound is from about 0.005 μg/ml to about 5 mg/ml.

8. The method of claim 1 wherein the stem cell is a human stem cell.

9. A method for modulating the proliferation or differentiation of a mammalian CD34⁺ or CD133⁺ progenitor cell comprising proliferating or differentiating said cell under conditions suitable for proliferation or differentiation and in the presence of a compound that inhibits PDE IV activity, wherein said compound is not a polypeptide, peptide, protein, hormone, cytokine, oligonucleotide, or nucleic acid.

10. The method of claim 9, wherein said progenitor cell is selected from the group consisting of a CD34⁺ progenitor cell and a CD133⁺ progenitor cell.

11. The method of claim 9, wherein said progenitor cells differentiate into CD34⁺CD38^{-CD}33⁺ or CD34⁺CD38^{-CD}33⁻ cells.
The method of claim 9, wherein said compound is a SelCID™ or prodrug thereof.

12. The method of claim 9 wherein said proliferation or differentiation is conducted in cell culture.

13. The method of claim 9, wherein said proliferation or differentiation is conducted within an individual.

14. The method of claim 13, wherein said progenitor cells are cells that have been transplanted into said individual.

15. The method of claim 9, wherein said compound is present in an amount sufficient to cause a detectable difference in said differentiation or proliferation relative to a control.

16. The method of claim 9, wherein said CD34⁺ or CD133⁺ progenitor cell has been cryopreserved and thawed prior to said differentiating.

17. A method for expanding a progenitor cell population in a mammalian subject, comprising administering a therapeutically effective amount of CD34⁺ progenitor cells and a compound that inhibits PDE IV activity to said mammalian subject, wherein said compound is not a polypeptide, peptide, protein, hormone, cytokine, oligonucleotide, or nucleic acid

18. The method of claim 17 wherein said CD34⁺ progenitor cells are differentiated in said mammalian subject.

19. The method of claim 17 wherein said CD34⁺ progenitor cells are administered to said mammalian subject in a cell preparation that is substantially free of red blood cells.

20. The method of claim 17 wherein said CD34⁺ progenitor cells are administered to said mammalian subject in a cell preparation that comprises bone marrow cells, placental cells, or cord blood cells.

21. The method of claim 17 wherein said CD34⁺ progenitor cells are administered to said mammalian subject in conjunction with a carrier.

22. The method of claim 17 wherein said CD34⁺ progenitor cells are CD34⁺CD38⁻ CD33⁺ or CD34⁺CD38⁻CD33⁻ progenitor cells.

23. The method of claim 17 wherein said CD34⁺ cell is a CD34⁺CD133⁺ progenitor cell.

24. The method of claim 17 wherein the progenitor cells express incorporated genetic material of interest.

25. A pharmaceutical composition comprising a mammalian stem cell and a pharmaceutically-acceptable carrier, wherein said stem cell has been contacted with a compound that inhibits PDE IV activity for a time sufficient to cause modulation of differentiation or proliferation of said stem cell, and wherein said compound is not a polypeptide, peptide, protein, hormone, cytokine, oligonucleotide, or nucleic acid.

26. The pharmaceutical composition of claim 25 wherein the stem cell is selected from the group consisting of an embryonic stem cell, a placental stem cell, a cord blood stem cell, a peripheral blood stem cell, and a bone marrow stem cell.

27. The pharmaceutical composition of claim 25 wherein said compound is a SelCID™ or prodrug thereof.

28. The pharmaceutical composition of claim 25 wherein said contacting step is conducted in cell culture.

29. The pharmaceutical composition of claim 25 wherein the concentration of said compound is from about 0.005 mg/ml to about 5 mg/ml.

30. The pharmaceutical composition of claim 25 wherein the stem cell is a human stem cell.

31. The pharmaceutical composition of claim 25 wherein the differentiation is differentiation into a hematopoietic cell.

32. The pharmaceutical composition of claim 25 wherein said hematopoietic cell is a CD34⁺ or CD38⁺ hematopoietic cell.

33. The pharmaceutical composition of claim 25 wherein the hematopoietic cell is a CD11b+ cell.

34. A pharmaceutical composition comprising isolated cord blood cells and an isolated population of white blood cells, wherein the white blood cells are generated by a method comprising differentiating stem cells under suitable conditions and in the presence of a compound that inhibits PDE IV activity, with the proviso that the compound is not a polypeptide, peptide, protein, hormone, cytokine, oligonucleotide, or nucleic acid, and isolating the white blood cells differentiated thereby.

35. The pharmaceutical composition of claim 34 wherein the compound is an imide or amide.

36. The pharmaceutical composition of claim 34 wherein the differentiating step is conducted in cell culture.

37. The pharmaceutical composition of claim 34 wherein the concentration of the compound is from about 0.005 μg/ml to about 5 mg/ml.

38. The pharmaceutical composition of claim 34 wherein the stem cell is a human stem cell.

39. The pharmaceutical composition of claim 34 wherein the stem cell is a progenitor cell.

40. The pharmaceutical composition of claim 39 wherein the progenitor cell is committed to a specific cell lineage.

41. The pharmaceutical composition of claim 39 wherein the progenitor cell is a hematopoietic progenitor cell.

42. A pharmaceutical composition comprising cultured CD34⁺ or CD133⁺ progenitor cells and a pharmaceutically-acceptable carrier, wherein said progenitor cells have been contacted within the first six days of culture with a compound that inhibits the activity of PDE IV, under conditions that promote proliferation and differentiation of said progenitor cells.

43. The pharmaceutical composition of claim 42 wherein said progenitor cells are collected and cryopreserved after six days of culture.

44. The pharmaceutical composition of claim 42 wherein said progenitor cells are CD34⁺CD38⁻CD34⁻ or CD34⁺CD38⁻CD34⁺ cells.

45. The pharmaceutical composition of claim 42 in which said compound is a SelCID™.

46. A method of transplanting a mammalian stem cell comprising:
(a) contacting said stem cell with a PDE IV-inhibitory compound to produce a treated stem cell, wherein said contacting is sufficient to modulate the differentiation of said stem cell; and
(b) administering said treated stem cell to an individual.

47. The method of claim 46, wherein step (b) comprises administering said treated stem cell in combination with untreated cells.

48. The method of claim 46 wherein the untreated cell is selected from the group consisting of an embryonic stem cell, a placental cell, a cord blood cell, a peripheral blood cell, and a bone marrow cell.

49. The method of claim 46, wherein said stem cell has been cryopreserved and thawed prior to said administering.

50. A method of transplanting a mammalian progenitor cell comprising:
(a) contacting said progenitor cell with a PDE VI-inhibitory compound to produce a treated progenitor cell, wherein said contacting is sufficient to modulate the differentiation of said progenitor cell; and
(b) administering said treated progenitor cell to an individual.

51. The method of claim 50, wherein step (b) comprises administering said treated progenitor cell in combination with untreated cells.

52. The method of claim 50 wherein the untreated cell is selected from the group consisting of an embryonic stem cell, a placental cell, a cord blood cell, a peripheral blood cell, and a bone marrow cell.

53. The method of claim 50, wherein said stem cell has been cryopreserved and thawed prior to said administering.

54. A method of treating an individual experiencing a condition comprising administering to said individual an agent selected from the group consisting of:
(a) a compound that inhibits PDE IV activity, wherein said compound is not a polypeptide, peptide, protein, hormone, cytokine, oligonucleotide, or nucleic acid;
(b) a stem cell differentiated in the presence of said compound; and
(c) a progenitor cell differentiated in the presence of said compound, wherein said agent detectably reduces or ameliorates said condition.

55. The method of claim 54, wherein said condition is selected from the group consisting of inflammation, heart disease, vascular disease, amylotrophic lateral sclerosis, a lysosomal storage disease, and diabetes.

56. The method of claim 54, wherein said agent comprises both a stem cell and compound that inhibits PDE IV activity, wherein said compound is not a polypeptide, peptide, protein, hormone, cytokine, oligonucleotide, or nucleic acid

57. A method of treating an individual comprising administering a therapeutically effective amount of white blood cells to said recipient mammalian subject, wherein said white blood cells are generated by a method comprising differentiating a stem cell under suitable conditions and in the presence of a compound that inhibits PDE IV activity, with the proviso that the compound is not a polypeptide, peptide, protein, hormone, cytokine, oligonucleotide, or nucleic acid.

58. The method of claim 57 wherein the stem cells are differentiated in vitro.

59. The method of claim 57 wherein the stem cells are differentiated in a postpartum perfused placenta.

60. The method of claim 57 wherein the white blood cells are administered to the individual in a cell preparation that is substantially free of red blood cells.

61. The method of claim 57 wherein the white blood cells are administered to the individual in a cell preparation which comprises cord blood cells.

62. The method of claim 57 wherein the white blood cells are administered to the individual in conjunction with a carrier.

63. The method of claim 57 wherein the white blood cells are administered to treat or repair a defect in the recipient mammalian subject.

64. The method of claim 63 wherein the defect is a hematopoietic or blood cell proliferation defect.

65. The method of claim 63 wherein the hematopoietic or blood cell proliferation defect is neutropenia or leukopenia.

66. The method of claim 63 wherein the white blood cells are administered systemically.

67. The method of claim 63 wherein the white blood cells are administered intravenously.

68. The method of claim 63 wherein the white blood cells express incorporated genetic material of interest.

69. The method of claim 57 wherein the white blood cells are allogeneic.

70. The method of claim 57 wherein the recipient mammalian subject is human.

71. A method of making a pharmaceutical composition, comprising:
(a) contacting CD34⁺ or CD133⁺ progenitor cells with a compound that inhibits PDE IV activity, wherein said progenitor cells are cultured for six days under culture conditions that allow proliferation and differentiation of said progenitor cells;
(b) collecting said cells after six days of culture; and
(c) placing said cells in a pharmaceutically-acceptable carrier.

72. The method of claim 71 wherein said contacting is performed on the first day of culture.

73. The method of claim 71, wherein said contacting is performed at least twice during said six days of culture.

74. The method of claim 71, wherein said compound is a SelCID™ or a prodrug thereof.

75. The method of claim 71, wherein said progenitor cells have been isolated from other blood cells prior to said culturing.

76. The method of claim 71, wherein said culture medium additionally contains GM-CSF and TNF-α.

77. The method of claim 74, wherein said SelCID™ or a prodrug thereof is present in a concentration of between 0.1 μM and 10.0 μM.

78. The method of claim 74 wherein said SelCID™ or a prodrug thereof is present at a concentration of 1.0 μM.

79. The method of claim 74, wherein said cells are cryopreserved after said collecting.

80. A pharmaceutical composition made by the process of claim 74.

81. A method for modulating the differentiation of a CD34⁺ or CD133⁺ progenitor cell comprising:
(a) providing a population of said progenitor cells under conditions such that differentiation can occur;
(b) contacting said progenitor cells with a compound, wherein said compound is a PDE IV inhibitor; and
(c) differentiating said progenitor cells under conditions suitable for differentiation, wherein said compound is placed in contact with said progenitor cells for at least part of the time said progenitor cells are differentiating.

82. The method of claim 81, wherein in step (b), said contacting is performed at any time between day 0 to day 6 of culture.

83. The method of claim 81, wherein in step (b), said contacting is performed at the start of the culture of said progenitor cells.

84. The method of claim 81, wherein in step (b), said contacting is performed after said progenitor cells have proliferated for at least two days.

85. The method of claim 81, wherein in step (b), said contacting is performed after said progenitor cells have proliferated for at least six days.

86. The method of claim 81, wherein said progenitor cells are CD34⁺ progenitor cells.

87. The method of claim 81, wherein said progenitor cells differentiate into cells exhibiting cell surface marker characteristics selected from the group consisting of:
a decrease in CD11c expression relative to a control;
a decrease in CD38 expression relative to a control;
a decrease in CD80 expression relative to a control;
a decrease in CD86 expression relative to a control;
a decrease in CD1a expression relative to a control;
a decrease in CD14 expression relative to a control;
a decrease in CD54^{bright} expression relative to a control;
a decrease in HLA-DR expression relative to a control;
an increase in CD15 expression relative to a control;
an increase in CD33 expression relative to a control;
an increase in CD54^{dim} expression relative to a control;
an increase in CD133 expression relative to a control; and
a combination of any of the above marker characteristics;
wherein said control is a CD34⁺ progenitor cell cultured under the same conditions as said progenitor cell in the absence of said compound.

88. The method of claim 81, wherein said progenitor cells differentiate into CD34⁺CD38⁻CD33⁺ or CD34⁺CD38⁻CD33⁻ cells.

89. The method of claim 81, wherein said PDE IV inhibitor is a SelCID™ or prodrug thereof.

90. A method of producing differentiated cells from CD34⁺ progenitor cells comprising culturing said cells in a culture medium that allows proliferation and differentiation, and contacting said progenitor cells with a SelCID™ or prodrug thereof.

91. The method of claim 90, wherein said contacting is performed on the first day of said culturing.

92. The method of claim 90, wherein said contacting takes place at least twice during the first six days of said culturing.

93. The method of claim 90, wherein said contacting takes place no earlier than said first day of culturing.

94. The method of claim 90, wherein said differentiated cell is a dendritic cell, a granulocyte, a CD34⁺CD38⁻CD33⁺ or a CD34⁺CD38⁻CD33⁻ cell.

95. The method of claim 90, wherein said CD34⁺ progenitor cell is a CD34⁺ CD133⁺ progenitor cell.

96. The method of claim 90, wherein said differentiated cells are isolated at day 6 of culture.

97. The method of claim 90, wherein said differentiated cells are isolated at day 12 of culture.

98. The method of claim 90, wherein said CD34⁺ cells have been isolated from other blood cells prior to said culturing.

99. The method of claim 90, wherein said culture medium additionally contains GM-CSF and TNF-α.

100. The method of claim 90, wherein said SelCID™ or prodrug thereof is present in a concentration of between 0.1 μM and 10.0 μM.

101. The method of claim 86 wherein said SelCID™ or prodrug thereof is present at a concentration of 1.0 μM.
